(19) United States
(12) Patent Application Publication
(10) Pub. No.: US 2022/0339222 A1
(43) Pub. Date: Oct. 27, 2022

(54) NEW ONCOLYTIC NEWCASTLE DISEASE VIRUSES AND RECOMBINANT NDV STRAINS

(71) Applicant: Arno THALLER, Markt Berolzheim (DE)

(72) Inventor: Arno THALLER, Markt Berolzheim (DE)

(21) Appl. No.: 17/642,985

(22) PCT Filed: Sep. 8, 2020
(86) PCT No.: PCT/EP2020/075032

§ 371 Date: Mar. 15, 2022

(30) Foreign Application Priority Data
Sep. 19, 2019 (EP) 19198308.9
Sep. 19, 2019 (EP) 19198311.3
Feb. 21, 2020 (EP) 20158903.3

(51) Int. Cl. A61K 35/768; C07K 14/005; C12N 7/00

(52) U.S. Cl. CPC A61K 35/768; C07K 14/005; C12N 7/00; C12N 2760/18121; C12N 2760/18122; C12N 2760/18132

(57) ABSTRACT

The invention relates to a novel Newcastle Disease Viruses (NDV) and transgene expressing Newcastle Disease Viruses (NDV), which have been demonstrated to possess significant oncolytic activity against mammalian cancers and an improved safety profile. The invention provides novel oncolytic viruses through the use of genetic engineering, including the transfer of foreign genes or parts thereof. The present invention also provides nucleic acids encoding a reverse genetically engineered (rg-)NDV comprising one or more of these foreign genes and having a mutation in the HN gene, said mutation allowing replication of said rgNDV in a cancer cell to a higher level than replication of an otherwise identical rgNDV not having said mutation in the HN gene, as well as a mutation in the F gene, said mutation resulting in a reduced ICPI value as compared to an otherwise identical rgNDV not having said at least one mutation in the F gene.

This invention relates to novel oncolytic Newcastle Disease Viruses (NDV) and recombinant Newcastle Disease Viruses, which have been demonstrated to possess significant oncolytic activity against mammalian cancers, especially selected from the specific cancers as described herein and/or mentioned in the claims, and which provide improved viral safety. The invention provides the development of novel and improved oncolytic agents through the use of genetic engineering, including the transfer of genes across species boundaries in order to produce improved genetically modified viruses carrying these transgenes.

### BACKGROUND

NDV is known as an oncolytic virus that is a virus for use in oncological treatment, preferably in the treatment of human subjects in need thereof. A number of RNA viruses, including NDV, reovirus, measles virus, and vesicular stomatitis virus (VSV), are members of this novel class of viruses being exploited as potential oncolytic agents. These oncolytic viruses are characterized in inherently replicating selectively within and killing tumor cells while leaving non-tumor cells unharmed. Accordingly these oncolytic viruses offer an attractive new tool for cancer therapy.

NDV derives its name from the site of the original outbreak in chickens at a farm near Newcastle-upon-Tyne in England in 1926. The virus is an economically important pathogen in multiple avian species and it is endemic in many countries. NDV is a member of the Avulavirus genus in the Paramyxoviridae family and is a non-segmented, negative-strand RNA virus, whose natural host range is limited to avian species; however, it is known to enter cells by binding to sialic acid residues present on a wide range of human and rodent cancer cells. The oncolytic property of NDV, i.e. to selectively replicate in and destroy tumor cells, while sparing normal cells, is believed to be based in part on defective antiviral responses in tumor cells. Normal cells, which are competent in launching an efficient antiviral response rapidly after infection, are able to inhibit viral replication before viral-mediated cell damage can be initiated. The sensitivity of NDV to interferon (IFN), coupled with defective IFN signalling pathways in tumor cells, provides one plausible mechanism whereby NDV can replicate exclusively within neoplastic tissue. The selective replication of NDV in human tumor cells may also be caused by several other mechanisms, including defects in activation of anti-viral signalling pathways, and activation of Ras signalling and/or expression of Rac1 (Schirrmacher, 2015, Expert Opin. Biol. Ther. 15:17 57-71).

Due to its tumor-specifity NDV is already under investigation as a safe clinical oncolytic virotherapy agent (Cassel et al., 1965, Cancer 1:863-888; Steiner et al., 2004, J. Clin. Oncol. 22:4272-4281; Schulze et al., 2009, Cancer Immunol. Immunother. 58:61-69; Csatary et al., 1999, Anticancer Res. 19:635-638; Pecora et al., 2002, J. Clin. Oncol. 20:2251-2266; Lorence et al., 2003, Curr. Opin. Mol. Ther. 5:618-624; Freeman et al., 2006, Mol. Ther. 13:221-228). The safety margin of NDV is a function of its inability to replicate in and kill normal human cells.

Typically, an oncolytic NDV strain is defined as an NDV for use in oncological virotherapy, preferably in the treatment of human subjects in need thereof. Multiple preclinical model studies have shown significant anti-tumor activity of natural and recombinant oncolytic NDV strains after varying treatment modalities. Promising results were noted in preclinical models of glioblastoma multiforme, anaplastic astrocytoma, leukemia, lymphoma, melanoma, neuroblastoma, osteosarcoma, rhabdomyosarcoma, Ewing's sarcoma, fibrosarcoma, pheochromocytoma, colon carcinoma, lung carcinoma, prostate carcinoma, breast carcinoma, ovary carcinoma, gastric carcinoma, mesothelioma, renal cell carcinoma, and head and neck carcinoma. One typically useful oncolytic NDV that has been extensively explored for use in the treatment of humans, and for which an advantageous safety and efficacy profile has been established in human trials, is a strain named MTH-68/H (Csatary L K, et al., J Neurooncol. 2004 March-April; 67(1-2):83-93). MTH-68/H therapy has been employed in a range of different tumors with success. MTH-68/H has been developed into a highly purified, lyophilized product, containing live, replication competent viral particles, grown to standardized titers. A Phase II clinical trial in humans was completed where the inhalatory mode of administration was used on patients suffering from a variety of advanced malignancies no longer responding to conventional cancer therapies. The study demonstrated relative efficacy, an overall improved quality of life, and a relatively benign side effect profile (Csatary L K, et al., Cancer detection and Prevention, 1993, 17(6):619-627). Success of treatment with MTH-68/H has also been shown in glioblastomas and anaplastic astrocytomas. Glioblastoma multiforme (GBM) is the most common primary brain tumor and by far the most aggressive form of glial tumors. This neoplasm has a poor prognosis, averaging six months to a year without therapy or about one-and-one-half years with conventional therapy. Four cases of advanced GBM and anaplastic astrocytoma were treated with the NDV viral product MTH-68/H after the conventional modalities of cancer therapy had failed. Results included survival rates of twelve and half to six years post-viral treatment for the surviving patients, and six years for one patient who has since died, after having discontinued treatment. Against all odds, these surviving patients returned to good quality of life and to a lifestyle that resemble their pre-morbid daily routines including giving birth to a healthy child and full professional activity. They have been able to enjoy good clinical health. These patients regularly received the MTH-68/H viral therapy for a number of years without interruption as a form of monotherapy once the classical modalities failed. The MRI and CT results have revealed an objective decrease in size of the tumors, in some cases the near total disappearance of the tumor mass.

NDV strains based on MTH-68/H having a mutation in the HN gene resulting in an increased replication capability in a cancer cell are disclosed in WO 2019/197275 A1. These NDV strains are also capable of expressing a foreign gene.

As reviewed by Zamarin and Palese (Future Microbiol. 2012 March; 7(3): 347-367), when compared to other oncolytic agents in development such as poxvirus, HSV-1, adenovirus, measles, and reovirus vectors, NDV as an oncolytic virus exhibits several advantages over other viruses for use in oncological treatment.

The first advantage is that NDV is an avian pathogen. This avoids the problems of preexisting immunity that can neutralize virus infectivity and pathogenicity of the virus in humans. These potential problems exist when using vaccinia, HSV-1, adenovirus, and measles vectors. Based on serological studies it seems that about 96% of the human population is seronegative for NDV, which avoids the problem of pre-existing immunity. The actual level of seropositivity in today's population may be even lower due to low human exposure to NDV, as the outbreaks of which are primarily limited to farm settings. To attest for viral safety, administration of virulent strains to humans has been shown to result in only mild to moderate adverse effects, with mild conjunctivitis, laryngitis, and flu-like symptoms as the only reported side effects. Natural human infections with highly virulent (avian) strains have been reported in the literature in farmers and laboratory workers and have been limited to mild symptoms such as conjunctivitis and laryngitis.

The second advantage offered by using NDV as an oncolytic agent is that NDV, similarly to other oncolytic viruses, possesses strong immunostimulatory properties that are the basis for oncolytic therapy being considered an immunotherapy. These properties include the induction of type I IFN and chemokines, upregulation of MHC and cell adhesion molecules, and facilitation of adhesion of lymphocytes and antigen-presenting cells (APCs) through expression of viral glycoproteins on the surface of infected cells. These properties have been shown to generate effective anti-tumor immune responses, which may persist long after clearance of the primary viral infection.

A third advantage is that the NDV genome has the plasticity to enable the incorporation and stable expression of foreign genes of relatively large size. Since these viruses replicate in the cytoplasm of the cell and not in the nucleus, an unintended integration of the foreign gene into the host genome is avoided. Such an unintended integration of foreign genes carried by an oncolytic agent into the host genome could be a safety problem with some DNA oncolytic vectors. Moreover, the absence of homologous RNA recombination ensures that foreign genes incorporated in and expressed from the NDV genome are stable for many serial passages in cell culture and in tumor cells.

Lastly, the ubiquitous nature of the NDV receptor allows for utilization of the virus against a wide variety of tumor types. The specificity of NDV for cancer cells due to their defects in antiviral pathways ensures viral safety.

Nevertheless, the full view of all clinical data generated to date in different trials of different NDV strains shows that there is much need for improvement in terms of percentage responders and magnitude of therapeutic effect. Also it is desired to further improve the viral safety. This need for improvement has spurred efforts to design improved recombinant NDV strains, which is the subject of the present patent application.

Similar to other paramyxoviruses in its family, the 15186, 15192 or 15198 nucleotide(nt)-long negative single-strand RNA genome of NDV encodes six genes including the nucleocapsid protein (NP), phosphoprotein (P), matrix protein (M), fusion protein (F), hemagglutinin-neuraminidase (HN), and RNA-dependent RNA polymerase (L) (Lamb R, Paramyxoviridae: the viruses and their replication. In: Knipe D, editor. Fields Virology, Lippincott Williams & Wilkins; 2007, pp. 1449-1496). The genes are separated by junction sequences that consist of three elements, known as gene start (GS), intergenic (IG), and gene-end (GE) motifs, which regulate mRNA transcription. In the P gene, a unique RNA editing mechanism adds non-templated G residues, resulting in the expression of V and W proteins that are colinear to the P protein in the amino-terminal end. The genomic RNA is bound in a ribonucleotide protein complex (RNP) consisting of NP, P, and L proteins and is surrounded by a lipid envelope containing three virus glycoprotein spikes, HN, M and F. An important factor that influences the extent of virulence of NDV in birds is the cleavage site of the F protein.

Pathogenic classification and virulence of NDV strains in birds generally correlates with their oncolytic properties in human cancer cells. Velogenic (high virulence) strains produce severe respiratory and nervous system signs, spread rapidly through chicken flocks, and can cause up to 90% mortality. Mesogenic (intermediate virulence) strains cause coughing, affect egg production and quality, and can result in up to 10% mortality. Lentogenic (low virulence) strains produce only mild symptoms with little if any mortality. Correspondingly, velogenic strains can efficiently carry out multicycle replication in multiple human cancer cells with effective and efficient cell lysis, lentogenic strains are more attenuated due to lack of activation of the FO protein, and mesogenic strains convey intermediate effects. On the basis of these observations in human cancers, NDV strains have been classified as either lytic or non-lytic, with velogenic and mesogenic viruses being lytic (high and low respectively), and lentogenic viruses in general being non-lytic. Early studies demonstrated that the lytic abilities of lentogenic NDV strains could be enhanced by the introduction of a polybasic cleavage site into their F proteins (Peeters et al, J. Virol. 1999; 73:5001-5009). Other NDV proteins including NP, P, V, HN, and L have also been shown to be implicated in virulence in birds. A deletion (18nt) introduced in the NP gene (Mebatsion T et al., J Virol. 2002 October; 76 (20):10138-46) resulted in the deletion of NP residues 443-460. The resulting mutant NDV propagated in embryonated specific-pathogen-free (SPF) chicken eggs to a level fully comparable to that of the parent virus.

In addition, a B-cell epitope of the S2 glycoprotein of murine hepatitis virus (MHV) was inserted in-frame. Recombinant NDV viruses properly expressing the introduced MHV epitope were successfully generated, demonstrating that the NP can be used as the insertion point in the NDV genome to insert foreign or transgenic sequences to be co-expressed with NDV during virus infection.

Several studies showed that type I IFN antagonist activity of NDV V protein is associated with pathogenicity in birds. In recombinant viruses possessing attenuating mutations or deletions in the V protein or in viruses expressing no or lower levels of V protein decreased pathogenicity in birds was observed (Huang et al., 2003, J Virol. 77:8676-8685; Mebatsion et al., 2001, J Virol. 75:420-428; Qiu et al., 2016, PLoS ONE 11(2): e0148560. doi: 10.1371/journal.pone.0148560; Elankumaran et al., 2010, J Virol. 84:3835-3844; Jang et al., 2010, Appl Microbiol Biotechnol. 85:1509-1520; Alamares et al., 2010, Virus Res. 147:153-157; Park et al., 2003, J Viro1.77:9522-9532). It was found to be sufficient to introduce a small (6 nt) deletion in the P gene to abolish expression of the V protein (Mebatsion et al., 2001).

Recently, two studies showed the role of the polymerase complex in viral pathogenesis, with L protein contributing to pathogenicity of the mesogenic Beaudette C strain, and NP, P, and L proteins contributing to pathogenicity of the velogenic Herts strain (Rout & Samal, 2008, J Virol. 82:7828-7836; Dortmans et al., 2010, J Virol. 84:10113-10120). In addition, several studies noted the contribution of the HN protein to virulence in birds (Römer-Oberdörfer et al., 2006, Avian Dis. 50:259-263; Paldurai et al., 2014, J Virol. 88: 8579-8596.) The HN (hemagglutinin-neuraminidase) protein of NDV is a multifunctional protein with receptor-recognition, hemagglutinin (HA) and receptor-destroying neuraminidase (NA) activities associated with the virus. HN is thought to possess both the receptor recognition of sialic acid at the termini of host glycoconjugates and the neuraminidase activity to hydrolyze sialic acid from progeny virion particles in order to prevent viral self-aggregation. It also recognizes sialic acid-containing receptors on cell surfaces and promotes the fusion activity of the F protein, thereby allowing the virus to penetrate the cell surface. Thus, the HN protein plays a critical role in viral infection of birds.

A recombinant NDV vector virus can be provided by a reverse genetics (rg) system, which has been known since 1999 (Peeters et al., 1999, J. Virol. 73:5001-5009). This system allows desired modification and engineering of NDV genomes. Indeed, various reports have shown the successful use of recombinant NDV vectors engineered to express various transgenes, i.e. foreign genes incorporated in the genome of the virus, with the goal of improving viral oncolytic efficacy (Vigil et al. Cancer Res. 2007, 67:8285-8292; Janke et al., 2007, Gene Ther. 14:1639-1649). As discussed above, the viral F protein of NDV is responsible for viral fusion with the cell membrane and for viral spread from host cell to host cell via formation of syncytia. The presence of the multi-basic amino acid cleavage site in the F protein enables protein cleavage and activation by a broad range or proteases and is known to be a determinant of virulence in velogenic NDV strains. In order to increase the oncolytic potency of a highly attenuated lentogenic Hitchner B1 NDV strain, a polybasic cleavage site was introduced into the F protein to generate rNDV/F3aa with mutated F protein. Altomonte et al. (Mol Ther. 2010 18:275-84) reported an oncolytic NDV vector virus with the mutated F-protein rNDV/F(L289A), harboring an L289A mutation within the F protein, which is required for virus entry and cell-cell fusion.

Due to compelling preclinical data implicating oncolytic NDV as an ideal candidate for cancer therapy, phase I and II clinical trials have been initiated. Several trials have shown that NDV is a safe and potentially therapeutically useful therapeutic agent, with no reports of significant adverse effects in patients beyond conjunctivitis or mild flu-like symptoms (Csatary et al., 1999, Anticancer Res. 19:635-638; Pecora et al., 2002, J. Clin. Oncol. 20:2251-2266; Lorence et al., 2003, Curr. Opin. Mol. Ther. 5, 618-624; Freeman et al., 2006, Mol. Ther. 13:221-228). Although the results of these clinical trials have been encouraging, the extent of clinical responses has not been strong and robust enough to consider bringing one of these initial NDV strains into advanced clinical development. Thus, strategies for improving therapeutic effectiveness while maintaining an acceptable safety profile of oncolytic NDV strains are needed. Thus there remains a clear need for improvement in therapeutic outcome by providing improved NDV strains, in particular NDV strains with enhanced killing effect without affecting—or even improving—its safety toward normal cells.

### DESCRIPTION OF THE PRESENT INVENTION

In a first aspect the present invention provides a novel Newcastle disease virus or novel NDV strains.

The NDV strains according to the present invention comprise a mutation in the viral HN gene, i.e. the nucleic acid sequence encoding the hemagglutinin-neuraminidase protein (HN protein), leading to a change in the amino acid sequence of the HN protein and providing the NDV with an increased replication capability in a cancer cell, preferably in a human cancer cell, as compared to an otherwise identical NDV not having the said mutation in the HN gene.

Preferably the mutation in the HN gene results in an unexpected beneficially increased replication capability of the NDV strain in cancer cells, preferably in human cancer cells, and more preferably results in a replication of said oncolytic NDV in a human cancer cell which is at least 2-fold higher, more preferably 2- to 10-fold higher, still more preferably 5- to 10-fold higher than an NDV strain not having said mutation in its viral HN gene. Preferably, said NDV strain comprises or is an oncolytic NDV already having an advantageous safety and efficacy profile in humans, such as the NDV strain MTH-68/H.

According to a preferred embodiment the NDV strain comprises a mutated HN gene and the encoded hemagglutinin-neuramidase (HN protein) with an amino acid substitution at position 277 to an amino acid with a hydrophobic side chain other than phenylalanine. Still more preferred is the amino acid phenylalanine (F) substituted to leucine (L) at amino acid position 277. Herein an oncolytic NDV having such a mutation is also identified as NDV^{F277L} to discern it from a parent NDV having a phenylalanine at amino acid position 277 of the HN gene.

The mutation in the HN gene is also identified as HN(F277L), wherein “HN” identifies the gene/nucleic acid sequence of the viral genome (i.e. in this example the HN gene), “277” identifies the amino acid position in the respective gene product (i.e. in this example the amino acid at position 277 of the HN protein), “F” or in general the letter before the number, identifies the amino acid originally present in the respective wild type sequence, and “L” or in general the letter following the number identifies the amino acid present in the NDV strain of the present invention. That is the amino acid at amino acid position 277 of the HN protein is “L”, which stands for leucine. A respective nomenclature is used for all other mutations described in the following.

In describing a protein or peptide formulation, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala: A: alanine; Asp: D: aspartic acid; Glu: E: glutamic acid; Phe: F: phenylalanine; Gly: G: glycine; His: H: histidine; Ile: I: isoleucine; Lys: K: lysine; Leu: L: leucine; Met: M: methionine; Asn: N: asparagine; Pro: P: proline; Gln: Q: glutamine; Arg: R: arginine; Ser: S: serine; Thr: T: threonine; Val: V: valine; Trp: W: tryptophan; Tyr: Y: tyrosine; Cys: C: cysteine.

According to the present invention the novel NDV strains comprise in addition to the above described mutation in the HN gene, mutations in the F gene and the thus encoded fusion protein (F protein). The mutated F gene encodes a fusion protein with an amino acid substitution at amino acid position 117 to an amino acid with a hydroxylated side chain, preferably where phenylalanine (F) is substituted to serine (S) at position 117 of the F gene product (also referred to as F(F117S) in the following), and/or with an amino acid substitution at amino acid position 190 to an amino acid with an aliphatic amino acid side chain, preferably where phenylalanine (F) is substituted to leucine (L) at position 190 of the F gene product (also referred to as F(F190L) in the following). The combination of both mutations at amino acid position 117 and 190 of the F gene is particularly preferred. In any case the nucleic acid sequence encoding the fusion protein encodes an amino acid substitution at position 289. This mutation is capable of improving the oncolytic potential of the respective NDV strain. Preferably the amino acid at position 289 of the F protein is an amino acid with an aliphatic side chain other than leucine (L). More preferably leucine (L) is substituted to alanine (A) at position 289 of the F protein (also referred to as F(L289A) in the following). Thus, the following mutations in the F protein are encompassed by the present invention: F(F117S)+F(L289A), F(F190L)+F(L289A, and F(F117S)+F(F190L)+F(L289A). I.e. the F(L289A) mutation is comprised in combination with at least one further amino acid substitution at position 117 or 190.

Within the meaning of the present invention the term “Newcastle disease virus” or “NDV” includes all known velogenic, mesogenic and lentogenic NDV strains.

According to the present invention mesogenic and lentogenic strains are preferred. Still more preferred is the NDV derived from NDV strain MTH-68/H.

A NDV strain derived from NDV strain MTH-68/H having a mutation in the HN gene product or HN protein at position 277, wherein the amino acid phenylalanine (F) is substituted to leucine (L) at position 277, and having mutations in the F gene resulting in a substitution of the amino acid phenylalanine (F) to the amino acid serine (S) at position 117 of the F gene product and a substitution of the amino acid leucine (L) to the amino acid alanine (A) at position 289 of the F gene product is referred to herein also as NDV-Mut HN(F277L)/F(F117S)/F(L289A). A NDV strain derived from NDV strain MTH-68/H having a mutation in the HN gene product or HN protein at position 277, wherein the amino acid phenylalanine (F) is substituted to leucine (L) at position 277, and having mutations in the F gene resulting in a substitution of the amino acid phenylalanine (F) to the amino acid leucine (L) at position 190 of the F gene product and a substitution of the amino acid leucine (L) to the amino acid alanine (A) at position 289 of the F gene product is referred to herein also as NDV-Mut HN(F277L)/F(F190L)/F(L289A). A NDV strain having the mutation in the HN gene and all three amino acid mutations in the F gene is referred to herein also as NDV-Mut HN(F277L)/F(F117S)/F(F190L)/F(L289A).

The following NDV strains derived from NDV strain MTH-68/H are provided by the present invention in a preferred embodiment:
NDV-Mut HN(F277L)/F(F117S)/F(L289A)
NDV-Mut HN(F277L)/F(F190L)/F(L289A)
NDV-Mut HN(F277L)/F(F117S)/F(F190L)/F(L289A)

Within the meaning of the present invention “derived from NDV strain MTH-68/H” is intended to mean that the derived NDV strain is encoded by a viral genome or comprises a viral genome having a nucleic acid sequence with a sequence identity of at least 70%, preferably of at least 75%, more preferably of at least 90%, more preferred of at least 95% to the nucleic acid sequence of the NDV strain MTH-68/H. In one embodiment the sequence identity is at least 99% or 100%. Any inserted sequences/transgenic constructs comprising nucleic acid sequences encoding one or more foreign genes comprised in the recombinant NDV strains are not considered in the sequence alignment, and the reference sequence for the sequence alignment is that of SEQ ID No. 1 of the sequence listing. A sequence variation is for example the substitution, insertion or deletion of at least one nucleotide, preferably the substitution, insertion or deletion of up to 20 nucleotides, still more preferably of up to 15 nucleotides. Any combination of substitution, insertion and deletion is also comprised by that definition, provided that the final construct possesses the desired characteristics. The variation may result in at least one amino acid substitution, wherein each amino acid substitution can be a conservative or a non-conservative amino acid substitution.

It has been surprisingly shown that the mutation in the HN gene results in a beneficially improved replication capability of the respective strain as compared to an otherwise identical NDV not having said mutation in the HN gene. The beneficially increased replication rate of the NDV not only results in higher number of virus particles capable of infecting and destroying cancer cells, but also results in a higher expression of a foreign gene carried by the respective recombinant NDV strains. As explained earlier, the respective gene products of the foreign gene results in an improved immunological response and thus an improved destruction of cancer cells.

Also it has been surprisingly shown that the at least one mutation, preferably both mutations (also referred to as “F2” herein), in the F gene at position 117 and 190 of the amino acid sequence of the resulting F protein beneficially improves the oncolytic potential of the respective NDV strains by improving the safety profile of the viruses in humans. Moreover, it has been surprisingly shown that the additional amino acid substitution at position 289 of the F protein in combination with at least one of the other mutations, preferably both mutations (also referred to as “F3” (=“F2”+L289A) herein) further improves the oncolytic potential of the NDV strain, i.e. the ability to infect and lyse cancer cells. That is the new NDV strains show improved oncolytic power and improved safety. The amino acid substitutions at the respective positions are as specified supra.

With regard to the viral safety profile, this in particular means that the intracerebral pathogenicity index (ICPI) is reduced by the mutations in the F gene at position 117 and/or 190, preferably by F2. The ICPI value indicates the the pathogenicity of a Newcastle disease virus. Preferably, the ICPI is reduced by at least 10%, more preferably by at least 20% and still more preferred by at least 30%, as compared to the identically determined ICPI value of a NDV strain not having the respective mutation(s) in the F gene. The ICPI can be determined according to the method set forth in World Organization for Animal Health (OIE) 2009, Chapter 2.3.14. Newcastle disease, pp. 576-589, in: Manual of Diagnostic Tests and Vaccines for Terrestrial Animals 2009, World Organization for Animal Health (OIE), Paris.

In a preferred embodiment of the present invention the mutated NDV strains comprise in addition to the above described mutation in the HN gene, and the mutations in the F gene at amino acid position 117 and/or 190 and 289, a mutation in the M gene and the thus encoded matrix protein (M protein). Still more preferred the mutated M gene encodes a matrix protein with an amino acid substitution at position 165 to an amino acid with an aromatic side chain. Still more preferred the amino acid glycine (G) at amino acid position 165 of the M gene is substituted to the amino acid tryptophane (W).

NDV strains comprising both a mutation in the HN gene and the M gene, as described supra, have been shown as being particularly suitable to infect cancer cells and to provide an enhanced replication rate within the cancer cells.

In a preferred embodiment of the present invention the mutated NDV strains comprise in addition to the above described mutation in the HN gene and the mutations in the F gene at amino acid positions 117 and/or 190 and 289 as specified above, at least one mutation in the L gene and the thus encoded RNA-dependent RNA polymerase protein, also referred to as L protein or L gene product herein. Still more preferred the mutated L gene encodes a RNA-dependent RNA polymerase protein with an amino acid substitution at position 757 to an amino acid with an aliphatic amino acid side chain other than valine (V), wherein preferably valine (V) is substituted to isoleucine (I) at position 757 of the L gene product, and/or with an amino acid substitution at position 1551 to an amino acid with a hydroxylated side chain, wherein preferably phenylalanine (F) is substituted to serine (S) at position 1551 of the L gene product, and/or with an amino acid substitution at position 1700 to an amino acid with an aliphatic side chain, preferably wherein arginine (R) is substituted to leucine (L) at position 1700 of the L gene product. Preferably the NDV genome comprises a mutated L gene encoding a L protein with the amino acid substitution at position 757, position 1551 and position 1700. These NDV strains may in addition also comprise the above described mutation in the M gene.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, having three mutations in the F gene, wherein the amino acid phenylalanine (F) is substituted to the amino acid serine (W) at position 117, the amino acid phenylalanine (F) is substituted to the amino acid leucine (L) at position 190, and the amino acid leucine (L) is substituted to the amino acid alanine (A) at position 289 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/F(L289A)/L(V757I)/L(F1551S)/L(R1700L) or MutHu2b or NDV-NoThaBene-2b. This NDV strain derived from NDV strain MTH-68/H is provided by the present invention in a preferred embodiment.

The three mutations in the F gene at position 117, 190 and 289 of the amino acid sequence of the resulting F protein and the three mutations in the L gene at position 757, 1551 and 1700 of the amino acid sequence of the resulting L protein, either alone or in combination, have been shown to beneficially improve the oncolytic potential of the respective NDV strains and the safety profile of these viruses in humans. The combinations include any combination of the referenced mutations in the F gene and L gene, and the F protein and L protein, respectively. That is any combination from three to six mutations. Particularly preferred is a combination of three mutations at amino acid position 117, 190 and 289 of the F gene (also referred to as “F3” herein) and a combination of all six mutations, that is the mutations at positions 117, 190 and 289 of the F gene product and at positions 757, 1551 and 1700 of the L gene product (also referred to as “F3L3” herein). The respective amino acid substitutions are as specified supra. The F3 and F3L3 mutations result in an improved oncolytic potential and a reduced intracerebral pathogenicity index (ICPI). Preferably, the ICPI is reduced by at least 10%, more preferably by at least 20% and still more preferred by at least 30%, as compared to the identically determined ICPI value of a NDV strain not having the respective mutations in the F gene and the L gene.

In addition to the mutation at position 277 of the HN gene product, the at least two mutations at positions 117 and/or 190 and 289 of F gene product, and preferably one or more of the above specified mutations at position 165 of the M gene product, and at positions 757 and/or 1551 and/or 1700 of the L gene product, a NDV according to the present invention may also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product.

Alternatively or in addition a NDV according to the present invention may comprise a mutation in the RNA-editing sequence of the P gene. More preferably, the mutation abolishes and/or decreases the expression of the V protein.

Table 1a shows a list of mutations for obtaining NDV variants with increased titer production and/or good or improved safety profile according to the present invention. The mutation in the HN gene and the at least two mutations in the F gene at amino acid position 117 and/or 190 and 289 is comprised in all NDV strains according to the present invention, while the mentioned mutations in the M gene, and L gene may or may not be present. If present, any possible combination of these mutations is encompassed by the present invention.

TABLE 1a

| Gene | Relevant amino acid |
|---|---|
| HN | F277L |
| M | G165W |
| F | F117S |
| F | F190L |
| F | L289A |
| L | V757I |
| L | F1551S |
| L | R1700L |
| L | Y1717H |
| L | E1910K |

Table 1b shows particularly preferred NDV variants with increased titer production and/or improved safety profile, and the parent NDV strain MTH-68/H. A “+” means that at the mentioned amino acid position the original (first mentioned) amino acid is substituted by the last mentioned amino acid, while “−” means that there is no amino acid substitution at the mentioned position, i.e. the first mentioned amino acid is present. For example and with regard to the HN gene “+” means that at amino acid position 277 phenylalanine (F) is substituted to leucine (L).

TABLE 1b

| | Relevant | | | | | | | | | | | | | | | | | | | |
|---|---|---|---|---|---|---|---|---|---|---|---|---|---|---|---|---|---|---|---|---|
| | amino | MTH- | | | | | | | | | | | | | | | | | | |
| Gene | acid | 68/H | | | | | | | | | | | | | | | | | | |
| HN | F277L | − | + | + | + | + | + | + | + | + | + | + | + | + | + | + | + | + | + | + |
| M | G165W | − | − | + | − | + | − | + | − | + | − | + | − | + | − | + | − | + | − | + |
| F | F117S | − | + | + | − | − | + | + | + | + | − | − | + | + | + | + | − | − | + | + |
| F | F190L | − | − | − | + | + | + | + | − | − | + | + | + | + | − | − | + | + | + | + |
| F | L289A | − | + | + | + | + | + | + | + | + | + | + | + | + | + | + | + | + | + | + |
| L | V757I | − | − | − | − | − | − | − | + | + | + | + | + | + | + | + | + | + | + | + |
| L | F1551S | − | − | − | − | − | − | − | + | + | + | + | + | + | + | + | + | + | + | + |
| L | R1700L | − | − | − | − | − | − | − | + | + | + | + | + | + | + | + | + | + | + | + |
| L | Y1717H | − | − | − | − | − | − | − | − | − | − | − | − | − | + | + | + | + | + | + |
| L | E1910K | − | − | − | − | − | − | − | − | − | − | − | − | − | + | + | + | + | + | + |

Table 1c shows all mutations in the F gene encompassed by the present invention. “+” means that at the mentioned amino acid position the original (first mentioned) amino acid is substituted by the last mentioned amino acid, while “−” means that there is no amino acid substitution at the mentioned position, i.e. the first mentioned amino acid is present. According to the present invention at least one of the mutations at amino acid position 117 or 190 is mandatory in addition to the mutation at amino acid position 289. The combination of both mutations at position 117 and 190 is particularly preferred.

TABLE 1c:

| | Relevant | | | |
|---|---|---|---|---|
| | amino | | | |
| Gene | acid | 2 | 2 | 3 |
| F | F117S | + | − | + |
| F | F190L | − | + | + |
| F | L289A | + | + | + |

Table 1 d shows preferred mutations in the L gene encompassed by the present invention. “+” means that at the mentioned amino acid position the original (first mentioned) amino acid is substituted by the last mentioned amino acid, while “−” means that there is no amino acid substitution at the mentioned position, i.e. the first mentioned amino acid is present.

TABLE 1d

| | Relevant | | | | | | | | | | | | | | | | | | | | | |
|---|---|---|---|---|---|---|---|---|---|---|---|---|---|---|---|---|---|---|---|---|---|---|
| | amino | | | | | | | | | | | | | | | | | | | | | |
| Gene | acid | 1 | 1 | 1 | 2 | 2 | 2 | 3 | 2 | 2 | 3 | 3 | 3 | 3 | 4 | 3 | 3 | 4 | 4 | 4 | 4 | 5 |
| L | V757I | + | − | − | + | + | − | + | + | − | − | + | + | − | + | + | − | − | + | + | − | + |
| L | F1551S | − | + | − | + | − | + | + | − | + | − | + | − | + | + | − | + | − | + | − | + | + |
| L | R1700L | − | − | + | − | + | + | + | − | − | + | − | + | + | + | − | − | + | − | + | + | + |
| L | Y1717H | − | − | − | − | − | − | − | + | + | + | + | + | + | + | + | + | + | + | + | + | + |
| L | E1910K | − | − | − | − | − | − | − | − | − | − | − | − | − | − | + | + | + | + | + | + | + |

In a preferred embodiment the parent NDV strain is encoded by and/or comprises the nucleic acid sequence according to SEQ ID No. 1 or parts thereof. The novel NDV strain according to the present invention can be encoded by and/or comprises the nucleic acids according to SEQ ID No. 2 or SEQ ID No. 3 or parts thereof. According to another preferred embodiment the parent NDV strain from which the novel NDV strains of the present invention are derived and the NDV strains of the present invention have a sequence identity of at least 70%, more preferably of at least 75%, more preferred of at least 90%, still more preferred of at least 95% to any one of SEQ ID No. 1 to 3. The sequences given by these SEQ ID numbers are as follows:
SEQ ID No. 1: Nucleic acid (cDNA) sequence of the Newcastle disease virus (NDV) strain MTH-68/H genome;
SEQ ID No. 2: Nucleic acid (cDNA) sequence of Newcastle disease virus strain Mut HN(F277L)/M(G165W)/F(F117S)/F(F190L)/F(L289A)/L(V 757I)/L(F1551S)/L(R1700L) genome having a mutation in the HN gene, a mutation in the M gene, three mutations in the F gene (F3) and three mutations in the L gene (L3) (=NDV-NoThaBene-2b genome).
SEQ ID No. 3: Nucleic acid (cDNA) sequence of Newcastle disease virus strain according to SEQ ID No. 2, but comprising in addition a 12 bp insert containing a AfeI restriction site between nucleotide positions 3134 and 3135 of SEQ ID No. 2.

The nucleic acid sequence of SEQ ID No. 3 differs from the nucleic acid sequence of SEQ ID No. 2 therein that a 12 bp insert containing unique AfeI restriction site between the P and M genes (between nucleotide position 3134-3135 of SEQ ID No. 2) has been inserted. This 12 bp sequence is for inserting a transgenic construct between the AGC and the GCT sequence. The sequence of the AfeI-site is AGCGCT. The nucleic acid sequence of this 12 bp insert is shown in SEQ ID No. 4 of the sequence listing.

A person skilled in the art is well aware of the fact that the nucleobase thymine in the nucleic acid sequence of DNA is replaced by the nucleobase uracil in the nucleic acid sequence of RNA.

In a preferred embodiment of the present invention the viral HN gene product has or comprises an amino acid sequence as identified in SEQ ID No. 32, and/or the viral M gene product preferably has or comprises an amino acid sequence as identified in SEQ ID No. 34. In addition it is preferred that a NDV strain according to the present invention comprises beside the viral HN gene product having an amino acid sequence as identified in SEQ ID No. 32, and/or the viral M gene product preferably having an amino acid sequence as identified in SEQ ID No. 34, a viral F gene product having or comprising an amino acid sequence as identified in SEQ ID No. 33 and/or a viral L gene product having or comprising an amino acid sequence as identified in SEQ ID No. 35. Also encompassed by the present invention are NDV strains comprising variants of the gene products according to SEQ ID No. 32 to 35.

The NDV as described supra can be a recombinant virus comprising a viral genome comprising a nucleic acid comprising a nucleic acid sequence encoding at least one foreign gene. The at least one foreign gene can be selected from the group consisting of:
A gene encoding an antibody directed to the protein PD-L1 or an antigen-binding part directed to the protein PD-L1 (anti-PD-L1), such as Atezolizumab or a variant or an antigen-binding part thereof. In one embodiment the antibody is Atezolizumab, an antigen-binding part of Atezolizumab, a variant of Atezolizumab or a variant of an antigen-binding part of Atezolizumab.
A gene encoding an antibody directed to the growth factor protein VEGF-A or an antigen-binding part directed to the growth factor protein VEGF-A (anti-VEGF-A), such as Bevacizumab or a variant or an antigen-binding part thereof. VEGF-A is a growth factor protein that stimulates angiogenesis in a variety of diseases, especially in cancer. Thus the antibody is capable of blocking angiogenesis. In one embodiment the antibody is Bevacizumab, an antigen-binding part of Bevacizumab, a variant of Bevacizumab or a variant of an antigen-binding part of Bevacizumab.
A gene encoding an antibody directed to the killer-cell immunoglobulin-like receptors (KIRs), in particular KIR2DL1/2L3, thereby regulating the killing function of the cells expressing the receptors, or an antigen-binding part directed to killer-cell immunoglobulin-like receptors (KIRs), in particular KIR2DL1/2L3, (anti-KIR). They work by regulating the killing function of the cells expressing the receptors. In one embodiment the antibody is Lirilumab, an antigen-binding part of Lirilumab, a variant of Lirilumab or a variant of an antigen-binding part of Lirilumab.
A gene encoding an antibody directed to and inhibiting LAG-3 or an antigen-binding part directed to directed to and inhibiting LAG-3 (anti-LAG-3). LAG-3 is a protein, which is present on the surface of T-cells. When the antibody or its antigen-binding part is bound to LAG-3 on T-cells, the T-cells are stimulated to attack cancer cells. In one embodiment the antibody is Relatlimab, an antigen-binding part of Relatlimab, a variant of Relatlimab or a variant of an antigen-binding part of Relatlimab. When bound to LAG-3 on T-cells, the T-cells are stimulated to attack cancer cells.
A gene encoding an antibody directed to NKG2A or an antigen-binding part directed to protein NKG2A (anti-NKG2A). Binding of the antibody to NKG2a prevents the binding of NKG2A to its ligand human leukocyte antigen-E (HLA-E), which induces an immune response against the cancer cells leading to their destruction. In one embodiment the antibody is Monalizumab, an antigen-binding part of Monalizumab, a variant of Monalizumab or a variant of an antigen-binding part of Monalizumab.
A gene encoding an antibody directed to the glucocorticoid-induced TNF-superfamily receptor (GITR), thereby blocking the interaction of GITR with its ligand, or an antigen-binding part directed to the glucocorticoid-induced TNF-superfamily receptor (GITR) (anti-GITR). In one embodiment the antibody is TRX518, an antigen-binding part of TRX518, a variant of TRX518 or a variant of an antigen-binding part of TRX518.
A gene encoding an antibody directed to and activating the protein OX40 (also known as CD134 or TNFRSF4), thereby increasing the number of T-cells, or an antigen-binding part directed to protein OX40 (anti-OX40). In one embodiment the antibody is BMS 986178, an antigen-binding part of BMS 986178, a variant of BMS 986178 or a variant of an antigen-binding part of BMS 986178.
A gene encoding the protein CD40 (cluster of differentiation 40), a part of CD40, a variant of CD40 or a variant of a part of CD40.
A gene encoding a membrane protein which is the receptor for the protein CD28, and which membrane protein is involved in the costimulatory signal essential for T-lymphocyte activation, or a part thereof which is the receptor for the protein CD28, and which membrane protein is involved in the costimulatory signal essential for T-lymphocyte activation. In one embodiment the membrane protein is CD80 (cluster of differentiation 80), a part of CD80, a variant of CD80 or a variant of a part of CD80.
A gene encoding a human interleukin 12 (hIL-12) protein, or a part thereof. In one embodiment the protein is (ns)hIL-12, a part of (ns)hIL-12, a variant of (ns)hIL-12 or a variant of a part of (ns)hIL-12. As used herein “(ns)” is intended to mean “non secreting”. That is the interleukin is preferred to be a non secreting human interleukin 12. A non secreting interleukin may be advantageous over a normal interleukin, as it may prevent that there is an uncontrolled immune response, for example in the form of a massive cytokine secretion, triggered by administration of the recombinant NDV strain.
A gene encoding a green fluorescent protein, which can be used for the localization and monitoring of cells, in particular cancer cells, or a part thereof. In one embodiment the protein is enhanced green fluorescent protein (eGFP), a part of enhanced green fluorescent protein (eGFP), a variant of enhanced green fluorescent protein (eGFP) or a variant of a part of eGFP.
A gene encoding an antibody capable of blocking checkpoint inhibition, such as an antibody directed against checkpoint inhibition protein PD-1 (programmed cell death protein 1, also known as CD279) or an antigen-binding part directed to protein PD-1 (anti-PD-1). They work by blocking a signal that prevents activated T cells from attacking the cancer upon expression in rNDV-expresser cells, thus allowing the immune system to clear the cancer. In one embodiment the antibody is Nivolumab, an antigen-binding part of Nivolumab, a variant of Nivolumab or a variant of an antigen-binding part of Nivolumab.
A gene encoding an antibody capable of blocking checkpoint inhibition, such as an antibody directed to the surface protein CTLA-4 or an antigen-binding part directed to protein CTLA-4 (anti-CTLA-4). In one embodiment the antibody is Ipilimumab, an antigen-binding part of Ipilimumab or a variant of Ipilimumab or a variant of an antigen-binding part of Ipilimumab.
A gene encoding a protein which improves the cellular immune response and improves the ability of T cells to enter tumor cells, or a part thereof which improves the cellular immune response and improves the ability of T cells to enter tumor cells. In one embodiment the protein is interleukin-12 (IL-12) or a part of interleukin-12 or a variant of interleukin-12 or a variant of a part of interleukin-12.
A gene encoding a protein with the ability to modulate the virus replication cycle, or a part thereof with the ability to modulate the virus replication cycle. In one embodiment the protein is the non-structural protein NS1 of influenza A virus or a part of the non-structural protein NS1 of influenza A virus or a variant of the non-structural protein NS1 of influenza A virus or a variant of a part of the non-structural protein NS1 of influenza A virus.
A gene encoding a protein with the ability to selectively induce apoptosis in human tumor cells, but not in normal human cells, or a part thereof with the ability to selectively induce apoptosis in human tumor cells, but not in normal human cells. In one embodiment the protein is Apoptin (VP3 from chicken anemia virus) or a part of Apoptin or a variant of Apoptin or a variant of a part of Apoptin.
A gene encoding a protein that reduces or inhibits IFN expression such as an IFN-beta receptor, or a part thereof that reduces or inhibits IFN expression such as an IFN-beta receptor. In one embodiment the protein is the viral protein B18R from vaccinia virus or a part of the viral protein B18R from vaccinia virus or a variant of the viral protein B18R from vaccinia virus or a variant of a part of the viral protein B18R from vaccinia virus. B18R is a homologue of the human IFN-β receptor. B18R acts as a decoy for IFN-β, thereby inhibiting activation by cell signaling of the antiviral host response in native cells, resulting in increased lytic activity in cancer cells.
A gene encoding an antibody, preferably an agonistic antibody, directed to CD28 or an antigen-binding part directed to CD28 (anti-CD28). CD28 (cluster of differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins. In one embodiment the antibody is Theralizumab, an antigen-binding part of Theralizumab, a variant of Theralizumab or a variant of an antigen-binding part of Theralizumab.
A gene encoding an antibody, preferably an agonistic antibody, directed to CD40 or an antigen-binding part directed to CD40 (anti-CD40). CD40 (cluster of differentiation 40) is a costimulatory protein found on antigen-presenting cells and is required for their activation. Thus, the antibody or its antigen-binding part is used to activate an anti-tumor T cell response via activation of the antigen-presenting cells.
A gene encoding an antibody, preferably an agonistic antibody, directed to CD80 or an antigen-binding part directed to CD80 (anti-CD80). CD80 (cluster of differentiation 80) is a membrane protein which is the receptor for the proteins CD28 and CTLA-4, and which membrane protein is involved in the costimulatory signal essential for T-lymphocyte activation. Thus, the antibody or its antigen-binding part is used to activate T-lymphocytes.
A gene encoding an antibody directed to CA 15-3 or an antigen-binding part directed to CA 15-3 (anti-CA 15-3). CA 15-3 is also known as the human gene MUC1.
A gene encoding an antibody directed to CA 19-9 or an antigen-binding part directed to CA 19-9 (anti-CA 19-9). CA 19-9 is a muzin which corresponds to the sialylated Lewisa blood group antigen.
A gene encoding an antibody directed to CA 125 or an antigen-binding part directed to CA 125 (anti-CA 125). CA 125 is also known as the human gene MUC16. An antibody or its antigen-binding part targeting the MUC16 protein results in a G2/M-phase growth arrest and tumour cell apoptosis. In one embodiment the antibody is Sofituzumab, an antigen-binding part of Sofituzumab, a variant of Sofituzumab or a variant of an antigen-binding part of Sofituzumab.
A gene encoding an antibody, preferably an antagonistic antibody, directed to EGFR or an antigen-binding part directed to EGFR (anti-EGFR). The epidermal growth factor receptor (EGFR) is a transmembrane protein. Permanent activation of the receptor results in an uncontrolled cell proliferation and prevents controlled cell death (apoptosis), which contributes to the malignant transformation of cells. The antagonistic antibody is directed against the extracellular domain of the EGFR and leads to an inhibition of ligand binding. In one embodiment the antibody is Cetuximab, an antigen-binding part of Cetuximab, a variant of Cetuximab or a variant of an antigen-binding part of Cetuximab.
A gene encoding an antibody, preferably an antagonistic antibody, directed to HER2 or an antigen-binding part directed to HER2 (anti-HER2). The antibody or its antigen-binding part is targeting the extracellular part of the HER2 receptor, which is overexpressed in certain cancers, and is intended to interrupt the growth-promoting signals. In one embodiment the antibody is Trastuzumab, an antigen-binding part of Trastuzumab, a variant of Trastuzumab or a variant of an antigen-binding part of Trastuzumab.
A gene encoding an antibody directed to nfP2X₇ or an antigen-binding part directed to nfP2X₇ (anti-nfP2X₇). The non-functioning P2X₇ receptor is a form of the ATP-gated P2X₇ receptor in which the E200 epitope is exposed for antibody binding. The nfP2X₇ is able to form a Ca²⁺ channel but not an apoptotic pore and is found on every type of cancer cell but no normal cells. In one embodiment the antibody is BIL=3s, an antigen-binding part of BIL=3s, a variant of BIL=3s or a variant of an antigen-binding part of BIL=3 s.
A gene encoding an antibody directed to PSMA or an antigen-binding part directed to PSMA (anti-PSMA). The prostate specific membrane antigen (PSMA) is a cell surface peptidase highly expressed by malignant prostate epithelial cells and vascular endothelial cells of numerous solid tumor malignancies. In one embodiment the antibody is J591, an antigen-binding part of J591, a variant of J591 or a variant of an antigen-binding part of J591.
A gene encoding an antibody, preferably an antagonistic antibody, directed to VEGFR or an antigen-binding part directed to VEGFR (anti-VEGFR). The vascular endothelial growth factor (VEGF) is involved in angiogenesis. The natural ligands comprise VEGF-A, VEGF-C and VEGF-D. Antagonistic antibodies targeting VEGF lead to inhibition of VEGF-mediated tumor angiogenesis. In one embodiment the antibody is Ramucirumab, an antigen-binding part of Ramucirumab, a variant of Ramucirumab or a variant of an antigen-binding part of Ramucirumab.
A gene encoding a protein that induces the process of apoptosis by binding to a death receptor, such as death receptor DR4 and/or death receptor DR5, or a part thereof that induces the process of apoptosis by binding to a death receptor, such as death receptor DR4 and/or death receptor DR5. In one embodiment the protein is TRAIL, a part of TRAIL, a variant of TRAIL or a variant of a part of TRAIL.
Any combination of these genes, parts or variants of these genes or parts thereof.

In following all these genes encoding the respective antibodies, antigen-binding parts thereof, proteins or parts thereof as well as any of the preferably mentioned antibodies, antigen-bindings parts, proteins, parts or variants thereof, are referred to in the following as “the foreign genes according to group A”. This group A also encompasses any combination of these genes, parts or variants of these genes or parts thereof.

The respective proteins or antibodies or parts thereof (i.e. the respective gene products) are also referred to in the following as “the foreign gene products according to group A”. This group also encompasses any combination of these gene products, parts or variants thereof.

An agonist is intended to mean a substance that activates signal transduction in the associated cell by occupying a receptor. Chemical compounds that bind to a receptor, but do not activate it, and thus block and inhibit it, are called antagonists.

The antibodies as mentioned above can be monoclonal antibodies, still more preferred monoclonal humanized antibodies, in a preferred embodiment.

The nucleic acid sequence comprising a nucleic acid sequence encoding at least one foreign gene, also referred to as “transgenic construct” or “inserted sequence” herein, can be or comprises a nucleic acid according to SEQ ID No. 5 or SEQ ID No. 30 or parts thereof. In another embodiment the transgenic construct can have a sequence identity of at least 70%, more preferably of at least 75%, more preferred of at least 90%, still more preferred of at least 95% to the respective sequence of any one of SEQ ID No. 5 to 30. The sequences given by these SEQ ID numbers are as follows:
SEQ ID No. 5: Nucleic acid sequence of a transgenic construct comprising a nucleic acid sequence encoding the anti-PD-L1 antibody Atezolizumab;
SEQ ID No. 6: Nucleic acid sequence of a transgenic construct comprising a nucleic acid sequence encoding an anti-VEGF-A antibody;
SEQ ID No. 7: Nucleic acid sequence of a transgenic construct comprising a nucleic acid sequence encoding an anti-KIR antibody;
SEQ ID No. 8: Nucleic acid sequence of a transgenic construct comprising a nucleic acid sequence encoding an anti-LAG-3 antibody;
SEQ ID No. 9: Nucleic acid sequence of a transgenic construct comprising a nucleic acid sequence encoding an anti-NKG2A antibody;
SEQ ID No. 10: Nucleic acid sequence of a transgenic construct comprising a nucleic acid sequence encoding an anti-GITR antibody;
SEQ ID No. 11: Nucleic acid sequence of a transgenic construct comprising a nucleic acid sequence encoding an anti-OX40 antibody;
SEQ ID No. 12: Nucleic acid sequence of a transgenic construct comprising a nucleic acid sequence encoding a CD80 protein;
SEQ ID No. 13: Nucleic acid sequence of a transgenic construct comprising a nucleic acid sequence encoding a (ns)hIL-12 protein;
SEQ ID No. 14: Nucleic acid sequence of a transgenic construct comprising a nucleic acid sequence encoding a eGFP;
SEQ ID No. 15: Nucleic acid sequence of a transgenic construct comprising a nucleic acid sequence encoding Nivolumab;
SEQ ID No. 16: Nucleic acid sequence of a transgenic construct comprising a nucleic acid sequence encoding an anti-CTLA-4 antibody;
SEQ ID No. 17: Nucleic acid sequence of a transgenic construct comprising a nucleic acid sequence encoding an interleukin-12 protein;
SEQ ID No. 18: Nucleic acid sequence of a transgenic construct comprising a nucleic acid sequence encoding a non-structural protein NS1;
SEQ ID No. 19: Nucleic acid sequence of a transgenic construct comprising a nucleic acid sequence encoding Apoptin;
SEQ ID No. 20: Nucleic acid sequence of a transgenic construct comprising a nucleic acid sequence encoding B18R;
SEQ ID No. 21: Nucleic acid sequence of a transgenic construct comprising a nucleic acid sequence encoding an anti-CD28 antibody;
SEQ ID No. 22: Nucleic acid sequence of a transgenic construct comprising a nucleic acid sequence encoding an anti-CA 15-3 antibody;
SEQ ID No. 23: Nucleic acid sequence of a transgenic construct comprising a nucleic acid sequence encoding an anti-CA 19-9 antibody;
SEQ ID No. 24: Nucleic acid sequence of a transgenic construct comprising a nucleic acid sequence encoding an anti-CA 125 antibody;
SEQ ID No. 25: Nucleic acid sequence of a transgenic construct comprising a nucleic acid sequence encoding an anti-EGFR antibody;
SEQ ID No. 26: Nucleic acid sequence of a transgenic construct comprising a nucleic acid sequence encoding an anti-HER2 antibody;
SEQ ID No. 27: Nucleic acid sequence of a transgenic construct comprising a nucleic acid sequence encoding an anti-nfP2X₇ antibody;
SEQ ID No. 28: Nucleic acid sequence of a transgenic construct comprising a nucleic acid sequence encoding an anti-PSMA antibody;
SEQ ID No. 29: Nucleic acid sequence of a transgenic construct comprising a nucleic acid sequence encoding an anti-VEGFR antibody;
SEQ ID No. 30: Nucleic acid sequence of a transgenic construct comprising a nucleic acid sequence encoding an anti-(ns)hIL-12;

The SEQ ID No. 31 of the sequence listing shows by way of example a transgenic NDV strain according to the present invention. Shown is a nucleic acid (cDNA) sequence of Newcastle disease virus strain Mut HN(F277L)/M(G165W)/F(F117S) /F(F190L)/F(L289A)/L(V757I)/L(F1551S)/L(R1700L)-Sofituzumab genome created by reverse genetics as disclosed herein, wherein the nucleic acid comprises a mutation in the HN gene, a mutation in the M gene, three mutations in the F gene (F3) and three mutations in the L gene (L3), and additionally encodes Sofituzumab.

The transgenic construct comprising the nucleic acid sequence encoding Sofituzumab is in this example inserted in the 12 nucleotide long sequence according to SEQ ID No. 4 of the sequence listing in the AfeI-site.

Instead of the nucleic acid sequence comprising a nucleic acid sequence encoding anti-CA 125, preferably Sofituzumab, any other nucleic acid sequence according to any one of SEQ ID No. 5 to 24 and SEQ ID No. 25 to 30 can be inserted to obtain a preferred transgenic NDV strain according to the present invention.

The foreign gene or part or the respective variant is/are expressed in the recombinant NDV strain of the present invention, when the virus is replicating in a suitable host or host cell. An expression of a gene means that the nucleic acid information encoded by the gene is translated in a respective amino acid sequence, which is the primary sequence for building up the respective protein or gene product. By way of example, the gene product of the HN gene is the HN protein.

In one embodiment the recombinant NDV can comprise any one of the foreign genes or parts or variants thereof according to group A. In another embodiment of the present invention a recombinant NDV can comprise in its viral genome at least two foreign genes or parts or variants thereof, which foreign genes or parts or variants are selected from the above mentioned foreign genes according to group A. Still more preferred the sequences comprising a nucleic acid sequence encoding a foreign gene can be any sequence according to SEQ ID No. 5 to 30 of the sequence listing or a variant thereof.

A “variant” of a starting nucleic acid is a nucleic acid that comprises a nucleic acid sequence different from that of the starting nucleic acid. Generally, a variant will possess at least 75% sequence identity, preferably at least 90% sequence identity, more preferably at least 95% sequence identity, and most preferably at least 98% sequence identity with the native nucleic acid. Variants of a nucleic acid may be prepared by introducing appropriate nucleotide changes into the nucleic acid. Such variants include, for example, deletions from, and/or insertions into and/or substitutions of residues within the nucleic acid sequence of the gene of interest. Any combination of deletion, insertion, and substitution is possibly made to arrive at the final construct, provided that the final construct possesses the desired characteristics. Methods for generating nucleic acid sequence variants are known to the skilled person.

A “variant” of a starting polypeptide or protein, e.g. an antibody or another protein, is a polypeptide or protein that comprises an amino acid sequence different from that of the starting polypeptide or protein. Generally, a variant will possess at least 75% sequence identity, preferably at least 90% sequence identity, more preferably at least 95% sequence identity, and most preferably at least 98% sequence identity with the native polypeptide or protein. Variants of a polypeptide or a protein may be prepared by introducing appropriate nucleotide changes into the nucleic acid encoding the polypeptide or protein. Such variants include, for example, deletions from, and/or insertions into and/or substitutions of residues within the amino acid sequence of the polypeptide/protein of interest. Any combination of deletion, insertion, and substitution is possibly made to arrive at the final construct, provided that the final construct possesses the desired characteristics. The amino acid changes also may alter post-translational processes of the polypeptide, such as changing the number or position of glycosylation sites. Methods for generating amino acid sequence variants of polypeptides are known to the skilled person.

Percentage sequence identity is determined after the best alignment of the sequence of interest with the respective reference sequence. The best alignment of the sequences may for example be produced by means of the Fitch et al., Proc. Natl. Acad. Sci. USA 80:1382-1386 (1983), version of the algorithm described by Needleman et al., J. Mol. Biol. 48:443-453 (1970), after aligning the sequences to provide for best homology, by means of the similarity search method of Pearson and Lipman, Proc. Natl Acad. Sci. USA 85, 2444 (1988), or by means of computer programs which use these algorithms (in particular BLASTN in Wisconsin Genetics Software Package, Genetics Computer Group, 575 Science Drive, Madison, Wis.). The percentage identity is calculated by determining the number of identical positions between the two sequences being compared, dividing this number by the number of positions compared and multiplying the result obtained by 100 so as to obtain the percentage identity between these two sequences. Preferably, sequence identity of a nucleotide sequence is determined using BLASTN, preferably BLASTN in standard settings as provided by the website of the U.S. National Library of Medicine “https://blast.ncbi.nlm.nih.gov”. Also preferably, sequence identity of an amino acid sequence is determined using BLASTP, preferably BLASTP in standard settings as provided by the website of the U.S. National Library of Medicine “https://blast.ncbi.nlm.nih.gov”. It is further preferred that the sequence identity is calculated over the entire length of the respective sequence.

As used herein, the term “part” means a fragment of a starting nucleic acid or amino acid sequence, which fragment is a made up of a consecutive sequence of the nucleotides or amino acids of the starting sequence. The part or fragment preferably comprises at least 10, more preferably at least 20 and preferably up to 500 nucleotides or amino acids. In any case a part according to the present invention is a functional part. That is nucleic acid or protein encoded by that nucleic acid must provide the desired function, e.g. an antigen-binding function or expression inhibiting function as specified herein, of the starting gene or protein.

The parent NDV strain carrying the at least one foreign gene comprises the mutations in the HN gene, the F gene and optionally the M gene and L gene as specified above. The parent NDV strain carrying the at least one foreign gene can be derived from the NDV strain MTH-68/H.

As mentioned above a NDV strain derived from NDV strain MTH-68/H having the respective mutations in the HN gene product or HN protein at position 277 and in the F gene resulting in a substitution of the amino acids at position 117 and/or 190 and 289 are referred to herein also as NDV-Mut HN(F277L)/F(F117S)/F(L289A), NDV-Mut HN(F277L)/F(F190L)/F(L289A) or NDV-Mut HN(F277L)/F(F117S)/(F190L)/F(L289A). The foreign gene or part or respective variant thereof carried by one of these strain is referred to as Atezolizumab, Bevacizumab, Lirilumab, Relatlimab, Monalizumab, TRX518, BMS 986178, CD40, CD80, (ns)hIL-12, eGFP, Nivolumab, Ipilimumab, IL-12, NS1, Apoptin, B18R, Theralizumab, anti-CD40, anti-CD80, anti-CA 15-3, anti-CA 19-9, Sofituzumab, Cetuximab, Trastuzumab, BIL=3s, J591, Ramucircumab or TRAIL in this shortcut for the strain.

Within the shortcut for the recombinant NDV strains as used herein a reference to e.g. Atezolizumab is intended to comprise the complete antibody, antigen-binding parts, and variants of the antibody or of antigen-binding parts, but also encompasses the more general form of an anti-PD-L1-antibody and antigen-binding parts of this antibody. A reference to Bevacizumab is again intended to comprise the complete antibody, antigen-binding parts and variants of the antibody or antigen-binding parts, but also the more general form of an antibody to the growth factor protein VEGF-A, and antigen-binding parts of this antibody. A reference to Lirilumab is intended to comprise the complete antibody, antigen-binding parts, and variants of the antibody or of antigen-binding parts, but also encompasses the more general form of an anti-KIR-antibody and antigen-binding parts of this antibody. A reference to Relatlimab is intended to comprise the complete antibody, antigen-binding parts, and variants of the antibody or of antigen-binding parts, but also encompasses the more general form of an anti-LAG-3-antibody and antigen-binding parts of this antibody. A reference to Monalizumab is intended to comprise the complete antibody, antigen-binding parts, and variants of the antibody or of antigen-binding parts, but also encompasses the more general form of an anti-NKG2A-antibody and antigen-binding parts of this antibody. A reference to TRX518 is intended to comprise the complete antibody, antigen-binding parts, and variants of the antibody or of antigen-binding parts, but also encompasses the more general form of an anti-GITR-antibody and antigen-binding parts of this antibody. A reference to BMS 986178 is intended to comprise the complete antibody, antigen-binding parts, and variants of the antibody or of antigen-binding parts, but also encompasses the more general form of an anti-OX40-antibody and antigen-binding parts of this antibody. A reference to CD40 is intended to comprise the complete protein, parts and variants of the protein. A reference to CD80 is intended to comprise the complete protein, parts and variants of the protein, as well as the more general form of a membrane protein which is the receptor for the protein CD28, and which membrane protein is involved in the costimulatory signal essential for T-lymphocyte activation, or a respective part thereof. A reference to (ns)hIL-12 is intended to comprise the complete protein, parts and variants of the protein, as well as the more general form of a human interleukin 12 (hIL-12), or a respective part thereof. A reference to eGFP is intended to comprise the complete protein, parts and variants of the protein, as well as the more general form of a green fluorescent protein, or a respective part thereof. A reference to Nivolumab is intended to comprise the complete antibody, antigen-binding parts, and variants of the antibody or of antigen-binding parts, but also encompasses the more general form of an anti-PD-1-antibody and antigen-binding parts of this antibody. A reference to Ipilimumab is again intended to comprise the complete antibody, antigen-binding parts and variants of the antibody or antigen-binding parts, but also the more general form of an anti-CTLA-4-antibody, and antigen-binding parts of this antibody. A reference to IL-12 is intended to comprise the complete protein, parts and variants of the protein, as well as the more general form of a protein which improves the cellular immune response and the ability of T cells to enter tumor cells, or a respective part thereof. A reference to NS1 is intended to comprise the complete protein, parts and variants of the protein, as well as the more general form of a protein with the ability to modulate the virus replication cycle, or a respective part thereof. A reference to Apoptin is intended to comprise the complete protein, parts and variants of the protein, as well as the more general form of a protein with the ability to selectively induce apoptosis in human tumor cells, but not in normal human cells, or a respective part thereof. A reference to B18R in this shortcut is intended to comprise the complete protein, parts and variants of the protein, as well as the more general form of a protein that reduces or inhibits IFN expression such as an IFN-beta receptor, or a respective part thereof. A reference to Theralizumab is intended to comprise the complete antibody, antigen-binding parts, and variants of the antibody or of antigen-binding parts, but also encompasses the more general form of an anti-CD28-antibody and antigen-binding parts of this antibody. A reference to anti-CD40 is intended to comprise the complete antibody, antigen-binding parts, and variants of the antibody or of antigen-binding parts. A reference to anti-CD80 is intended to comprise the complete antibody, antigen-binding parts, and variants of the antibody or of antigen-binding parts. A reference to anti-CA 15-3 is intended to comprise the complete antibody, antigen-binding parts, and variants of the antibody or of antigen-binding parts according to SEQ ID. 22, but also encompasses the more general form of an anti-CA 15-3-antibody and antigen-binding parts of this antibody. A reference to anti-CA 19-9 is intended to comprise the complete antibody, antigen-binding parts, and variants of the antibody or of antigen-binding parts according to SEQ ID. 23, but also encompasses the more general form of an anti-CA 19-9-antibody and antigen-binding parts of this antibody. A reference to Sofituzumab is intended to comprise the complete antibody, antigen-binding parts, and variants of the antibody or of antigen-binding parts, but also encompasses the more general form of an anti-CA 125-antibody and antigen-binding parts of this antibody. A reference to Cetuximab is intended to comprise the complete antibody, antigen-binding parts, and variants of the antibody or of antigen-binding parts, but also encompasses the more general form of an anti-EGFR-antibody and antigen-binding parts of this antibody. A reference to Trastuzumab is intended to comprise the complete antibody, antigen-binding parts, and variants of the antibody or of antigen-binding parts, but also encompasses the more general form of an anti-HER2-antibody and antigen-binding parts of this antibody. A reference to BIL=3s is intended to comprise the complete antibody, antigen-binding parts, and variants of the antibody or of antigen-binding parts, but also encompasses the more general form of an anti-nfP2X₇-antibody and antigen-binding parts of this antibody. A reference to J591 is intended to comprise the complete antibody, antigen-binding parts, and variants of the antibody or of antigen-binding parts, but also encompasses the more general form of an anti-PSMA-antibody and antigen-binding parts of this antibody. A reference to Ramucirumab is intended to comprise the complete antibody, antigen-binding parts, and variants of the antibody or of antigen-binding parts, but also encompasses the more general form of an anti-VEGFR-antibody and antigen-binding parts of this antibody. A reference to TRAIL is intended to comprise the complete protein, parts and variants of the protein, as well as the more general form of a protein with the ability to induce the process of apoptosis by binding to a death receptor, such as death receptor DR4 and/or death receptor DR5, or a respective part thereof. A reference to the foreign gene in this shortcut is also intended to comprise the sequence of the transgenic construct comprising the respective foreign gene as identified in any of the nucleic acid sequences according to SEQ ID No. 5 to 30 of the sequence listing or a variant thereof.

The following recombinant NDV strains are provided by the present invention in a preferred embodiment:
NDV-Mut HN(F277L)/F(F117S)/F(F190L)/F(L289A)-Atezolizumab
NDV-Mut HN(F277L)/F(F117S)/F(F190L)/F(L289A)-Bevacizumab
NDV-Mut HN(F277L)/F(F117S)/F(F190L)/F(L289A)-Lirilumab
NDV-Mut HN(F277L)/F(F117S)/F(F190L)/F(L289A)-Relatlimab
NDV-Mut HN(F277L)/F(F117S)/F(F190L)/F(L289A)-Monalizumab
NDV-Mut HN(F277L)/F(F117S)/F(F190L)/F(L289A)-TRX518
NDV-Mut HN(F277L)/F(F117S)/F(F190L)/F(L289A)-BMS 986178
NDV-Mut HN(F277L)/F(F117S)/F(F190L)/F(L289A)-CD40
NDV-Mut HN(F277L)/F(F117S)/F(F190L)/F(L289A)-CD80
NDV-Mut HN(F277L)/F(F117S)/F(F190L)/F(L289A)-(ns)hIL-12
NDV-Mut HN(F277L)/F(F117S)/F(F190L)/F(L289A)-eGFP
NDV-Mut HN(F277L)/F(F117S)/F(F190L)/F(L289A)-Nivolumab
NDV-Mut HN(F277L)/F(F117S)/F(F190L)/F(L289A)-Ipilimumab
NDV-Mut HN(F277L)/F(F117S)/F(F190L)/F(L289A)-IL-12
NDV-Mut HN(F277L)/F(F117S)/F(F190L)/F(L289A)-NS1
NDV-Mut HN(F277L)/F(F117S)/F(F190L)/F(L289A)-Apoptin
NDV-Mut HN(F277L)/F(F117S)/F(F190L)/F(L289A)-B18R
NDV-Mut HN(F277L)/F(F117S)/F(F190L)/F(L289A)-Theralizumab
NDV-Mut HN(F277L)/F(F117S)/F(F190L)/F(L289A)-anti-CD40
NDV-Mut HN(F277L)/F(F117S)/F(F190L)/F(L289A)-anti-CD80
NDV-Mut HN(F277L)/F(F117S)/F(F190L)/F(L289A)-anti-CA 15-3
NDV-Mut HN(F277L)/F(F117S)/F(F190L)/F(L289A)-anti CA 19-9
NDV-Mut HN(F277L)/F(F117S)/F(F190L)/F(L289A)-Sofituzumab
NDV-Mut HN(F277L)/F(F117S)/F(F190L)/F(L289A)-Cetuximab
NDV-Mut HN(F277L)/F(F117S)/F(F190L)/F(L289A)-Trastuzumab
NDV-Mut HN(F277L)/F(F117S)/F(F190L)/F(L289A)-BIL=3s
NDV-Mut HN(F277L)/F(F117S)/F(F190L)/F(L289A)-J591
NDV-Mut HN(F277L)/F(F117S)/F(F190L)/F(L289A)-Ramucircumab
NDV-Mut HN(F277L)/F(F117S)/F(F190L)/F(L289A)-TRAIL

Thus, according to one embodiment of the present invention a recombinant NDV strain, which can be derived from NDV strain MTH-68/H, comprises as a foreign gene at least one gene selected from the foreign genes according to group A, and has a mutation in the HN gene, preferably resulting in a substitution of the amino acid phenylalanine (F) to leucine (L) at position 277 of the HN gene product, and at least two mutation in the F gene, resulting in a substitution of the amino acid phenylalanine (F) to the amino acid serine (S) at position 117 of the F gene product, and/or resulting in a substitution of the amino acid phenylalanine (F) to the amino acid leucine (L) at position 190 of the F gene product and resulting in a substitution of the amino acid leucine (L) to the amino acid alanine (A) at position 289 of the F gene product. The combination of both mutations at amino acid position 117 and 190 of the F gene product is particularly preferred. As explained supra, other substitutions at these amino acid positions are also comprised within the present invention. In one embodiment the recombinant NDV strain can additionally comprise a mutation in the M gene, preferably resulting in a substitution of the amino acid glycine (G) to the amino acid tryptophane (W) at position 165 of the M gene product.

The recombinant and mutated NDV strains can comprise in addition to the above described mutation in the HN gene and the at least two mutations in the F gene at amino acid positions 117 and/or 190 and 289 as specified above, at least one mutation in the L gene and the thus encoded RNA-dependent RNA polymerase protein at amino acid positions 757 and/or 1551 and/or 1700 as specified above. These recombinant NDV strains may in addition also comprise the above described mutation in the M gene. As it relates to preferred and more preferred embodiments of the substitutions at amino acid positions 117, 190 and 289 of the F gene, and amino acid positions 757, 1551 and 1700 of the L gene reference to above disclosure with regard to the novel NDV strains per se is made. The same preferred and more preferred embodiments or amino acids apply for the recombinant NDV strains.

According to one embodiment of the present invention a recombinant NDV strain comprises as a foreign gene at least one gene selected from the foreign genes according to group A, and comprises a mutation in the HN gene, preferably resulting in a substitution of the amino acid phenylalanine (F) to leucine (L) at amino acid position 277 of the HN gene, two or more of the above described mutations in the F gene, preferably resulting in a substitution of the amino acid phenylalanine (F) to the amino acid serine (S) at position 117 of the gene product and/or resulting in a substitution of the amino acid phenylalanine (F) to the amino acid leucine (L) at position 190 of the F gene product and resulting in a substitution of the amino acid leucine (L) to the amino acid alanine (A) at position 289 of the F gene product, and at least one mutation in the L gene, preferably one resulting at amino acid position 757 to an amino acid with an aliphatic amino acid side chain other than valine (V), preferably where valine (V) is substituted to isoleucine (I) at position 757 of the L gene product, and/or with an amino acid substitution at position 1551 to an amino acid with a hydroxylated side chain, preferably where phenylalanine (F) is substituted to serine (S) at position 1551 of the L gene product, and/or with an amino acid substitution at position 1700 to an amino acid with an aliphatic side chain, preferably where arginine (R) is substituted to leucine (L) at position 1700 of the L gene product, preferably wherein the NDV comprises a mutated L gene and the encoded L protein with the amino acid substitution at position 757, position 1551 and position 1700. These recombinant NDV strains may in addition also comprise the above described mutation in the M gene, preferably resulting in a substitution of the amino acid glycine (G) to the amino acid tryptophane (W) at position 165 of the M gene product. As explained supra, other substitutions at these amino acid positions are also comprised within the present invention.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, having three mutations in the F gene, wherein the amino acid phenylalanine (F) is substituted to the amino acid serine (W) at position 117, the amino acid phenylalanine (F) is substituted to the amino acid leucine (L) at position 190 and the amino acid leucine (L) is substituted to the amino acid alanine (A) at position 289 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/F(L289A)/L(V757I)/L(F1551S)/L(R1700L) or MutHu2b or NDV-NoThaBene-2b. The foreign gene or part or variant thereof carried by this strain is referred to as Atezolizumab, Bevacizumab, Lirilumab, Relatlimab, Monalizumab, TRX518, BMS 986178, CD40, CD80, (ns)hIL-12, eGFP, Nivolumab, Ipilimumab, IL-12, NS1, Apoptin, B18R, Theralizumab, anti-CD40, anti-CD80, anti-CA 15-3, anti CA 19-9, Sofituzumab, Cetuximab, Trastuzumab, BIL=3s, J591, Ramucircumab or TRAIL in this shortcut for the strain. Within these shortcuts for the recombinant NDV strains a reference to the respective foreign gene is intended to have the meaning as already defined above. The following NDV strains are provided by the present invention in a particularly preferred embodiment:
NDV-Mut HN(F277L)/M(G165W)/F(F117S)/F(F190L)/F(L289A)/L(V757I)/L(F1551S)/L(R1700L)-Atezolizumab
NDV-Mut HN(F277L)/M(G165W)/F(F117S)/F(F190L)/F(L289A)/L(V757I)/L(F1551S)/L(R1700L)-Bevacizumab
NDV-Mut HN(F277L)/M(G165W)/F(F117S)/F(F190L)/F(L289A)/L(V757I)/L(F1551S)/L(R1700L)-Lirilumab
NDV-Mut HN(F277L)/M(G165W)/F(F117S)/F(F190L)/F(L289A)/L(V757I)/L(F1551S)/L(R1700L)-Relatlimab
NDV-Mut HN(F277L)/M(G165W)/F(F117S)/F(F190L)/F(L289A)/L(V757I)/L(F1551S)/L(R1700L)-Monalizumab
NDV-Mut HN(F277L)/M(G165W)/F(F117S)/F(F190L)/F(L289A)/L(V757I)/L(F1551S)/L(R1700L)-TRX518
NDV-Mut HN(F277L)/M(G165W)/F(F117S)/F(F190L)/F(L289A)/L(V757I)/L(F1551S)/L(R1700L)-BMS 986178
NDV-Mut HN(F277L)/M(G165W)/F(F117S)/F(F190L)/F(L289A)/L(V757I)/L(F1551S)/L(R1700L)-CD40
NDV-Mut HN(F277L)/M(G165W)/F(F117S)/F(F190L)/F(L289A)/L(V757I)/L(F1551S)/L(R1700L)-CD80
NDV-Mut HN(F277L)/M(G165W)/F(F117S)/F(F190L)/F(L289A)/L(V757I)/L(F1551S)/L(R1700L)-(ns)hIL-12
NDV-Mut HN(F277L)/M(G165W)/F(F117S)/F(F190L)/F(L289A)/L(V757I)/L(F1551S)/L(R1700L)-eGFP
NDV-Mut HN(F277L)/M(G165W)/F(F117S)/F(F190L)/F(L289A)/L(V757I)/L(F1551S)/L(R1700L)-Nivolumab
NDV-Mut HN(F277L)/M(G165W)/F(F117S)/F(F190L)/F(L289A)/L(V757I)/L(F1551S)/L(R1700L)-Ipilimumab
NDV-Mut HN(F277L)/M(G165W)/F(F117S)/F(F190L)/F(L289A)/L(V757I)/L(F1551S)/L(R1700L)-IL-12
NDV-Mut HN(F277L)/M(G165W)/F(F117S)/F(F190L)/F(L289A)/L(V757I)/L(F1551S)/L(R1700L)-NS1
NDV-Mut HN(F277L)/M(G165W)/F(F117S)/F(F190L)/F(L289A)/L(V757I)/L(F1551S)/L(R1700L)-Apoptin
NDV-Mut HN(F277L)/M(G165W)/F(F117S)/F(F190L)/F(L289A)/L(V757I)/L(F1551S)/L(R1700L)-B18R
NDV-Mut HN(F277L)/M(G165W)/F(F117S)/F(F190L)/F(L289A)/L(V757I)/L(F1551S)/L(R1700L)-Theralizumab
NDV-Mut HN(F277L)/M(G165W)/F(F117S)/F(F190L)/F(L289A)/L(V757I)/L(F1551S)/L(R1700L)-anti-CD40
NDV-Mut HN(F277L)/M(G165W)/F(F117S)/F(F190L)/F(L289A)/L(V757I)/L(F1551S)/L(R1700L)-anti-CD80
NDV-Mut HN(F277L)/M(G165W)/F(F117S)/F(F190L)/F(L289A)/L(V757I)/L(F1551S)/L(R1700L)-anti-CA 15-3
NDV-Mut HN(F277L)/M(G165W)/F(F117S)/F(F190L)/F(L289A)/L(V757I)/L(F1551S)/L(R1700L)-anti CA 19-9
NDV-Mut HN(F277L)/M(G165W)/F(F117S)/F(F190L)/F(L289A)/L(V757I)/L(F1551S)/L(R1700L)-Sofituzumab
NDV-Mut HN(F277L)/M(G165W)/F(F117S)/F(F190L)/F(L289A)/L(V757I)/L(F1551S)/L(R1700L)-Cetuximab
NDV-Mut HN(F277L)/M(G165W)/F(F117S)/F(F190L)/F(L289A)/L(V757I)/L(F1551S)/L(R1700L)-Trastuzumab
NDV-Mut HN(F277L)/M(G165W)/F(F117S)/F(F190L)/F(L289A)/L(V757I)/L(F1551S)/L(R1700L)-BIL=3s
NDV-Mut HN(F277L)/M(G165W)/F(F117S)/F(F190L)/F(L289A)/L(V757I)/L(F1551S)/L(R1700L)-J591
NDV-Mut HN(F277L)/M(G165W)/F(F117S)/F(F190L)/F(L289A)/L(V757I)/L(F1551S)/L(R1700L)-Ramucircumab
NDV-Mut HN(F277L)/M(G165W)/F(F117S)/F(F190L)/F(L289A)/L(V757I)/L(F1551S)/L(R1700L)-TRAIL.

According to another embodiment of the present invention a recombinant NDV according to the present invention may also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product. Alternatively or in addition a recombinant NDV according to the present invention may comprise a mutation in the RNA-editing sequence of the P gene. More preferably, the mutation abolishes and/or decreases the expression of the V protein. For further details reference to the above disclosure, for example tables 1a to 1 d, concerning the novel NDV strain per se is made.

A person skilled in the art is well aware of the fact that the nucleobase thymine in the nucleic acid sequence of DNA is replaced by the nucleobase uracil in the nucleic acid sequence of RNA.

The present invention also provides a nucleic acid comprising the nucleic acid sequence encoding the NDV strains of the present invention.

In describing nucleic acid formulation, structure and function herein, reference is made to any of a group of polymeric nucleotides such as DNA or RNA. Nucleic acid is composed of either one or two chains of repeating units called nucleotides, which nucleotides consist of a nitrogen base (a purine or pyrimidine) attached to a sugar phosphate. In the present specification, nucleotide residues are identified by using the following abbreviations. Adenine residue: A; guanine residue: G; thymine residue: T; cytosine residue: C; uracil residue: U. Also, unless explicitly otherwise indicated, the nucleotide sequences of nucleic acid are identified from 5′-terminal to 3′-terminal (left to right terminal), the 5′-terminal being identified as a first residue.

The nucleic acid according to the present invention comprises a nucleic acid sequence (HN gene) encoding a HN protein having a mutation allowing replication of the NDV in a cancer cell to a higher level than replication of an otherwise identical NDV not having said the mutation in the HN gene.

In a preferred embodiment of the present invention the nucleic acid comprises a nucleic acid sequence encoding a hemagglutinin-neuramidase protein with an amino acid substitution at position 277 to an amino acid with a hydrophobic side chain other than phenylalanine. Still more preferred the nucleic acid sequence encodes for a hemagglutinin-neuramidase protein referred to as HN^{F277L}. That is the amino acid phenylalanine (F) at position 277 of the hemagglutinin-neuramidase is substituted to leucine (L).

In addition a nucleic acid according to the present invention comprises a nucleic acid sequence (F gene) encoding a fusion protein with an amino acid substitution at position 117 and/or position 190 and position 289. With regard to amino acid position 117 the amino acid is substituted to an amino acid with a hydroxylated side chain. Most preferred the mutated F gene encodes F^{F117S}. That is the amino acid phenylalanine (F) at position 117 of the F gene product is substituted to the amino acid serine (S) at the respective position. With regard to the amino acid substitution at position 190 the amino acid is changed to an amino acid with an aliphatic side chain. Most preferred the mutated F gene encodes F^{F190L}. That is the amino acid phenylalanine (F) at position 190 of the F gene product is substituted to the amino acid leucine (L) at the respective position. The combination of both mutations at amino acid position 117 (F^{F117S}) and 190 (F^{F190L}) is particularly preferred. With regard to amino acid position 289 the amino acid is substituted to an amino acid with an aliphatic side chain other than leucine (L). Preferably the mutated F gene encodes F^{L289A}. That is the amino acid leucine (L) at position 289 of the F gene product is substituted to the amino acid alanine (A) at the respective position.

In a particular preferred embodiment the nucleic acid is derived from the NDV strain MTH-68/H.

The nucleic acid can also comprise a nucleic acid sequence (M gene) encoding a mutated M protein. Preferably the mutated M gene encodes a matrix protein with an amino acid substitution at position 165 to an amino acid with an aromatic side chain. Most preferred the mutated M gene encodes M^{G165W}. That is the amino acid glycine (G) at position 165 of the M gene product is substituted to the amino acid tryptophane (W) at the respective position. In one embodiment of the present invention the mutation in the M gene is comprised in combination with the mutations in the HN gene and in the F gene as specified supra.

In a particular preferred embodiment the nucleic acid is derived from the NDV strain MTH-68/H and comprises a mutation in the HN gene resulting in an amino acid with a hydrophobic side chain other than phenylalanine at amino acid position 277, a mutation in the M gene resulting in a matrix protein with an amino acid substitution at position 165 to an amino acid with an aromatic side chain, and at least two mutation in the F gene resulting in a fusion protein with an amino acid substitution at position 117 to an amino acid with a hydroxylated side chain and/or an amino acid substitution at position 190 to an amino acid with an aliphatic side chain and an amino acid substitution at position 289 to an amino acid with an aliphatic side chain other than leucine (L). Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), and the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L) and the amino acid leucine (L) at position 289 is substituted to the amino acid alanine (A). The combination of the three mutations at amino acid position 117 (F^{F117S}), position 190 (F^{F190L}) and position 289 (F^{L289A}) of the F gene is particularly preferred.

The nucleic acid can of the present invention can, in addition to the above described mutation in the HN gene, particularly the mutation resulting in an amino acid substitution at position 277 of the HN gene product as specified supra, and in addition to the above described at least two mutations in the F gene, particularly the mutation resulting in an amino acid substitution at position 117 (F^{F117S}) and/or at position 190 (F^{F190L}) and position 289 (F^{L289A}) of the F gene product as specified supra, optionally in combination with the above described mutation on the M gene, particularly the mutation resulting in an amino acid exchange at position 165 of the M gene product as specified supra, also comprises a nucleic acid sequence (L gene) having at least one mutation. Preferably the mutated L gene encodes for a L protein with at least one amino acid substitution at amino acid position 757, 1551 and/or 1700. At position 757 the amino acid may be changed to an amino acid with an aliphatic side chain other than valine (V). Most preferred the mutated L gene encodes L^{V757I}. That is the amino acid valine (V) at position 757 of the L gene product is substituted to the amino acid isoleucine (I) at the respective position. At position 1551 the amino acid may be changed to an amino acid with a hydroxylated side chain. Most preferred the mutated L gene encodes L^{F1551S}. That is the amino acid phenylalanine (F) at position 1551 of the L gene product is substituted to the amino acid serine (S) at the respective position. At position 1700 the amino acid may be changed to an amino acid with an aliphatic side chain. Most preferred the mutated L gene encodes L^{R1700L}. That is the amino acid arginine (R) at position 1700 of the L gene product is substituted to the amino acid leucine (L) at the respective position. The mutations at amino acid positions 757, 1551 and 1700 of the L gene may be present alone or in each possible combination, i.e. position 757 and/or position 1551 and/or position 1700. The combination of all three mutations in the L gene (L3) is particularly preferred. In one embodiment of the present invention the at least one mutation in the L gene, preferably all three mutation at the respective amino acid positions 757, 1551 and 1700, is/are comprised in combination with the mutation in the HN gene as specified supra, the two or three mutations in the F gene as specified supra, and optionally, also in combination with the mutation in the M gene as specified supra.

In a particular preferred embodiment the nucleic acid is derived from the NDV strain MTH-68/H and comprises a mutation in the HN gene resulting in an amino acid with a hydrophobic side chain other than phenylalanine at position 277 of the respective amino acid sequence, a mutation in the M gene resulting in a matrix protein with an amino acid substitution at position 165 to an amino acid with an aromatic side chain, mutations of the F gene resulting in a fusion protein with an amino acid substitution at position 117 to an amino acid with a hydroxylated side chain and/or an amino acid substitution at position 190 to an amino acid with an aliphatic side chain and an amino acid substitution at position 289 to an amino acid with an aliphatic side chain other than leucine (L), and at least one mutation in the L gene resulting in a L protein with an amino acid substitution at position 757 to an amino acid with an aliphatic side chain other than valine (V), and/or an amino acid substitution at position 1551 to an amino acid with a hydroxylated side chain, and/or an amino acid substitution at position 1700 to an amino acid with an aliphatic side chain. More preferred, the mutated L gene results in a L protein comprising the three amino acid substitutions at positions 757, 1551 and 1700. Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L) and the amino acid leucine (L) at position 289 is substituted to the amino acid alanine (A), and the mutated L gene encodes for a L protein in which the amino acid valine (V) at position 757 is substituted to the amino acid isoleucine (I), and/or the amino acid phenylalanine (F) at position 1551 is substituted to the amino acid serine (S), and/or the amino acid arginine (R) at position 1700 is substituted to the amino acid leucine (L). Still more preferred the L protein comprises all three mutations at positions 757, 1551 and 1700.

Also within the scope of the present invention are those nucleic acids comprising, in addition to the mutated HN gene and to the mutated F gene encoding at least two mutations at amino acid position 117 and/or 190 and 289, preferably in combination with a mutated M gene with one or more mutation, and the mutated L gene, as specified supra, a nucleic acid sequence (L gene) encoding, in addition to the at least one mutation at amino acid position 757 and/or 1551 and/or 1700, at least one an amino acid substitution at position 1717 and/or at position 1910 of the L protein, and/or a mutated P gene as specified supra.

In one embodiment of the present invention the nucleic acid consists of or comprises at least one of the nucleic acids according to SEQ ID No. 2 or 3 or parts thereof. In another preferred embodiment the nucleic acid of the present invention has a sequence identity of at least 75%, particularly of at least 90%, more particularly of at least 95% to any of the sequences according to SEQ ID No. 2 or 3.

The nucleic acid as described supra can additionally comprise a transgenic construct or inserted nucleic acid sequence comprising a nucleic acid sequence encoding at least one protein or antibody or a part thereof. Such a nucleic acid is also referred to as a recombinant nucleic acid herein. The at least one protein or antibody or antigen-binding part can be selected from the foreign gene products according to group A. The transgenic construct can be a nucleic acid sequence according to any of SEQ ID No. 5 to 30 of the sequence listing or a variant or part thereof.

In one embodiment of the present invention the nucleic acid or recombinant nucleic acid is derived from a NDV as described supra.

The skilled artisan knows how to use the disclosed sequences to produce synthetic DNAs (e.g. vectors, in particular in rg-NDV vectors), and how to use the synthetic DNAs to reconstruct and express the viral genome. Thus, the skilled artisan is enabled to produce virus particles and the respective nucleic acids by using the information disclosed herein.

In the following a method for obtaining recombinant viral genomes will be described in detail. A person skilled in the art knows how to transfer the disclosed method for obtaining mutated viral genomes not comprising a transgenic construct.

Recombinant viral genomes, which can be used to rescue virus particles, can for example be obtained by a method called ‘reverse genetics’. Therefore, in another aspect the present invention also provides a method (herein also called ‘reverse genetics’) for providing cloned full-length cDNA of the NDV strains and recombinant NDV strains according to the present invention. In a further embodiment, the invention also provides infectious virus particles obtained from said full-length cDNA of the NDV strains or recombinant NDV strains according to the present invention. These NDV strains have an unexpected beneficial replication capability in cell cultures, eggs or animals that are used to propagate the virus for pharmaceutical purposes, and have good or even an improved viral safety profile, giving production advantages to such improved rgNDV strains. Furthermore, the higher replication rate is also particularly useful to obtain the desired oncolytic effects in vivo in that more virus particles will be produced for subsequent rounds of infection of cancer cells that were missed in the first round of infection. What is more, if the NDV strain is recombinant, higher levels (yields) of the foreign gene or the foreign genes encoded by the transgenic construct will be achieved for providing the desired enhanced oncolytic effects in vivo. Thus, by modifying the HN protein within a transgene-expressing NDV higher yields of the transgenic construct protein can be obtained, which is very useful in anti-tumor immunotherapy. By modifying the F protein, and preferably also the L protein, as specified supra, beside the oncolytic potential also the viral safety can be improved.

The NDV strains according to the present invention can be obtained by a reverse genetic method for preparing an rgNDV, optionally encoding at least one foreign gene, preferably at least one foreign gene according to group A or comprising a nucleic acid sequence according to any of SEQ ID No. 5 to 30 of the sequence listing, and having improved replication in a cancer cell, and good or even improved viral safety, over a parent NDV.

The method according to the present invention comprises providing a nucleic acid construct comprising a HN gene with a mutation, particularly wherein the mutation in the HN gene leads to a change in the expression of the hemagglutinin-neuraminidase, wherein the amino acid, particularly phenylalanine (F), in position 277 is substituted, particularly wherein the amino acid in position 277 is substituted to an amino acid with a hydrophobic side chain, more particularly wherein the amino acid in position 277 is substituted to leucine (L) at position 277 of the HN gene product/HN protein, and comprising a F gene encoding a fusion protein/F protein with an amino acid substitution at position 117 to an amino acid with a hydroxylated side chain and/or with an amino acid substitution at position 190 to an amino acid with an aliphatic side chain and an amino acid substitution at position 289 to an amino acid with an aliphatic side chain other than leucine. Still more preferred is a substitution of the amino acid phenylalanine (F) at position 117 of the F gene product to the amino acid serine (S) and/or a substitution of the amino acid phenylalanine (F) at position 190 of the F gene product to the amino acid leucine (L) and a substitution of the amino acid leucine (L) at position 289 of the F gene product to the amino acid alanine (A). The combination of all three mutations in the F gene is particularly preferred.

The method further comprises a step of providing a nucleic acid encoding a rgNDV further comprising a transgenic construct encoding at least one of the foreign gene products according to group A or comprising a nucleic acid sequence according to any of SEQ ID No. 5 to 30 of the sequence listing.

In the next step of the claimed method the nucleic acid construct with said mutations in the HN gene and the at least two mutations in the F gene at amino acid position 117 and/or position 190 and position 289 (optionally with further mutations, in particular those as specified herein) is incorporated in the nucleic acid encoding a rgNDV comprising the transgenic construct in order to obtain a full length cDNA of the NDV, which additionally comprises a transgenic construct as specified supra. In a preferred embodiment this method step results in a nucleic acid as disclosed supra.

The nucleic acid encoding a rgNDV further comprising a transgenic construct as specified supra can be obtained by generating sub-genomic cDNA fragments and assembling the full length cDNA of the recombinant NDV from these fragments.

Following these method steps the thus obtained nucleic acid encoding a mutated and optionally recombinant rgNDV according to the present invention is used to produce infectious rgNDV particles, which replication characteristics and expression rates of the encoded foreign protein(s) in cancer cells are compared with the replication characteristics and expressions rates of the parent NDV used for designing the recombinant and mutated NDV strain. Also the ICPI values of the produced infectious rgNDV particles are compared with the parent NDV.

Finally, said rgNDV is selected for further use, when it shows an improved replication characteristic over the parent NDV and good or even an improved viral safety, as well as a sufficient expression of the gene product or gene products of the at least one foreign genes.

If it is intended to introduce further to the mutation in the HN gene and the at least two mutations in the F gene at amino acid position 117 and/or position 190 and position 289, at least one further mutation in a viral gene, preferably the M gene, the L gene and/or the P gene, the selected recombinant and mutated rgNDV comprising a transgenic construct as specified supra and a mutated HN gene is used for incorporating a nucleic acid construct encoding a mutated M gene, L gene or P gene, and the method for introducing the mutations in the HN gene and F gene is repeated respectively.

Alternatively there is already provided a nucleic acid encoding a rgNDV with the transgenic construct and carrying a mutation in the M gene, F gene, L gene or P gene, particularly wherein said mutation is capable of improving oncolytic potential of said rgNDV and/or viral safety.

According to a preferred embodiment of the present invention the mutation in the M gene comprises or is a mutation which encodes a matrix protein with an amino acid substitution at position 165 to an amino acid with an aromatic side chain. Still more preferred is a substitution of the amino acid glycine (G) at position 165 of the M gene product to the amino acid tryptophane (W).

According to a still further preferred embodiment of the present invention the at least one mutation in the L gene comprises or is a mutation which encodes a L protein with an amino acid substitution at position 757 to an amino acid with an aliphatic side chain other than valine, and/or with an amino acid substitution at position 1551 to an amino acid with a hydroxylated side chain, and/or with an amino acid substitution at position 1700 to an amino acid with an aliphatic side chain. Still more preferred is a substitution of the amino acid valine (V) at position 757 of the L gene product to the amino acid isoleucine (I) and/or a substitution of the amino acid phenylalanine (F) at position 1551 of the L gene product to the amino acid serine (S), and/or a substitution of the amino acid arginine (R) at position 1700 of the L gene product to the amino acid leucine (L). Optionally, the L gene may also comprise a mutation which encodes a L protein with an amino acid substitution at position 1717 to an amino acid with an aromatic side chain other than tyrosine, preferably a substitution of the amino acid tyrosine (Y) at position 1717 of the L gene product to the amino acid histidine (H), and/or a mutation which encodes a L protein with an amino acid substitution at position 1910 to an amino acid with a basic side chain, preferably a substitution of the amino acid glutamic acid (E) at position 1910 of the L gene product to the amino acid lysine (K).

According to another preferred embodiment of the present invention the mutation is in the RNA-editing sequence of the P gene. More preferably, the mutation abolishes and/or decreases the expression of the V protein.

It is also within the scope of the present invention that the viral genome comprises nucleic acid sequences comprising a combination of two or more mutations, which are selected from a mutation in the HN gene, a mutation in the M gene, at least two mutations in the F gene, at least one mutation in the L gene, and a mutation in the P gene. For the details of the respective mutations, reference to the detailed disclosure of each of these mutations given supra and in tables 1 is made.

The invention also provides an isolated, recombinant nucleic acid encoding a rgNDV obtainable by a method for preparing an rgNDV having improved replication in a cancer cell in comparison to that of a parent NDV, as described supra.

In one preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (a F277L mutation) allowing replication of said rgNDV in a human cancer cell to a higher level than replication of an otherwise identical rgNDV having the amino acid phenylalanine (F) at position 277 in the HN gene product, and with mutations in the F gene resulting in a change of the amino acid phenylalanine (F) at amino acid position 117 of the fusion protein into serine (S) and/or resulting in a change of the amino acid phenylalanine (F) at amino acid position 190 of the fusion protein into leucine (L) and resulting in a change of the amino acid leucine (L) at amino acid position 289 of the fusion protein into alanine (A). These mutations are provided in combination with a transgenic construct encoding any of the foreign genes or parts or variants as specified supra. The rgNDV may further comprise at least one or more mutation(s) selected from a mutation in the M gene, preferably G165W, a mutation at amino acid position 757, 1551, 1700, 1717 and/or 1910 in the L gene, preferably V757I, F1551S, R1700L, Y1717H and/or E1910K, and a mutation in the P gene, preferably the mutation in the P gene abolishes and/or decreases the expression of the V protein.

According to a particularly preferred embodiment the nucleic acid used to engineer a NDV genome or a recombinant NDV genome is derived from the NDV strain MTH-68/H. Thus the present invention provides in one embodiment nucleic acids, preferably obtainable by the above specified method, which are or comprise
rgNDV-Mut HN(F277L)/F(F117S)/F(F190L)/F(L289A)
rgNDV-Mut HN(F277L)/F(F117S)/F(F190L)/F(L289A)-Atezolizumab
rgNDV-Mut HN(F277L)/F(F117S)/F(F190L)/F(L289A)-Bevacizumab
rgNDV-Mut HN(F277L)/F(F117S)/F(F190L)/F(L289A)-Lirilumab
rgNDV-Mut HN(F277L)/F(F117S)/F(F190L)/F(L289A)-Relatlimab
rgNDV-Mut HN(F277L)/F(F117S)/F(F190L)/F(L289A)-Monalizumab
rgNDV-Mut HN(F277L)/F(F117S)/F(F190L)/F(L289A)-TRX518
rgNDV-Mut HN(F277L)/F(F117S)/F(F190L)/F(L289A)-BMS 986178
rgNDV-Mut HN(F277L)/F(F117S)/F(F190L)/F(L289A)-CD40
rgNDV-Mut HN(F277L)/F(F117S)/F(F190L)/F(L289A)-CD80
rgNDV-Mut HN(F277L)/F(F117S)/F(F190L)/F(L289A)-(ns)hIL-12
rgNDV-Mut HN(F277L)/F(F117S)/F(F190L)/F(L289A)-eGFP
rgNDV-Mut HN(F277L)/F(F117S)/F(F190L)/F(L289A)-Nivolumab
rgNDV-Mut HN(F277L)/F(F117S)/F(F190L)/F(L289A)-Ipilimumab
rgNDV-Mut HN(F277L)/F(F117S)/F(F190L)/F(L289A)-IL-12
rgNDV-Mut HN(F277L)/F(F117S)/F(F190L)/F(L289A)-NS1
rgNDV-Mut HN(F277L)/F(F117S)/F(F190L)/F(L289A)-Apoptin
rgNDV-Mut HN(F277L)/F(F117S)/F(F190L)/F(L289A)-B18R
rgNDV-Mut HN(F277L)/F(F117S)/F(F190L)/F(L289A)-Theralizumab
rgNDV-Mut HN(F277L)/F(F117S)/F(F190L)/F(L289A)-anti-CD40
rgNDV-Mut HN(F277L)/F(F117S)/F(F190L)/F(L289A)-anti-CD80
rgNDV-Mut HN(F277L)/F(F117S)/F(F190L)/F(L289A)-anti-CA 15-3
rgNDV-Mut HN(F277L)/F(F117S)/F(F190L)/F(L289A)-anti CA 19-9
rgNDV-Mut HN(F277L)/F(F117S)/F(F190L)/F(L289A)-Sofituzumab
rgNDV-Mut HN(F277L)/F(F117S)/F(F190L)/F(L289A)-Cetuximab
rgNDV-Mut HN(F277L)/F(F117S)/F(F190L)/F(L289A)-Trastuzumab
rgNDV-Mut HN(F277L)/F(F117S)/F(F190L)/F(L289A)-BIL=3s
rgNDV-Mut HN(F277L)/F(F117S)/F(F190L)/F(L289A)-J591
rgNDV-Mut HN(F277L)/F(F117S)/F(F190L)/F(L289A)-Ramucircumab
rgNDV-Mut HN(F277L)/F(F117S)/F(F190L)/F(L289A)-TRAIL.

In another embodiment the present invention provides nucleic acids, preferably obtainable by the above specified method, which are or comprise
rgNDV-Mut HN(F277L)/M(G165W)/F(F117S)/F(F190L)/F(L289A)/L(V757I)/L(F1551S)/L(R1700L) (=rgNDV-NoThaBene-2b)
rgNDV-Mut HN(F277L)/M(G165W)/F(F117S)/F(F190L)/F(L289A)/L(V757I)/L(F1551S)/L(R1700L)-Atezolizumab (=rgNDV-NoThaBene-2b-Atezo-lizumab)
rgNDV-Mut HN(F277L)/M(G165W)/F(F117S)/F(F190L)/F(L289A)/L(V757I)/L(F1551S)/L(R1700L)-Bevacizumab (=rgNDV-NoThaBene-2b-Bevacizumab)
rgNDV-Mut HN(F277L)/M(G165W)/F(F117S)/F(F190L)/F(L289A)/L(V757I)/L(F1551S)/L(R1700L)-Lirilumab (=rgNDV-NoThaBene-2b-Lirilumab)
rgNDV-Mut HN(F277L)/M(G165W)/F(F117S)/F(F190L)/F(L289A)/L(V757I)/L(F1551S)/L(R1700L)-Relatlimab (=rgNDV-NoThaBene-2b-Relatlimab)
rgNDV-Mut HN(F277L)/M(G165W)/F(F117S)/F(F190L)/F(L289A)/L(V757I)/L(F1551S)/L(R1700L)-Monalizumab (=rgNDV-NoThaBene-2b-Monalizumab)
rgNDV-Mut HN(F277L)/M(G165W)/F(F117S)/F(F190L)/F(L289A)/L(V757I)/L(F1551S)/L(R1700L)-TRX518 (=rgNDV-NoThaBene-2b-TRX518)
rgNDV-Mut HN(F277L)/M(G165W)/F(F117S)/F(F190L)/F(L289A)/L(V757I)/L(F1551S)/L(R1700L)-BMS 986178 (=rgNDV-NoThaBene-2b-BMS 986178)
rgNDV-Mut HN(F277L)/M(G165W)/F(F117S)/F(F190L)/F(L289A)/L(V757I)/L(F1551S)/L(R1700L)-CD40 (=rgNDV-NoThaBene-2b-CD40)
rgNDV-Mut HN(F277L)/M(G165W)/F(F117S)/F(F190L)/F(L289A)/L(V757I)/L(F1551S)/L(R1700L)-CD80 (=rgNDV-NoThaBene-2b-CD80)
rgNDV-Mut HN(F277L)/M(G165W)/F(F117S)/F(F190L)/F(L289A)/L(V757I)/L(F1551S)/L(R1700L)-(ns)hIL-12 (=rgNDV-NoThaBene-2b-(ns)hIL-12)
rgNDV-Mut HN(F277L)/M(G165W)/F(F117S)/F(F190L)/F(L289A)/L(V757I)/L(F1551S)/L(R1700L)-eGFP (=rgNDV-NoThaBene-2b-eGFP)
rgNDV-Mut HN(F277L)/M(G165W)/F(F117S)/F(F190L)/F(L289A)/L(V757I)/L(F1551S)/L(R1700L)-Nivolumab (=rgNDV-NoThaBene-2b-Nivolumab)
rgNDV-Mut HN(F277L)/M(G165W)/F(F117S)/F(F190L)/F(L289A)/L(V757I)/L(F1551S)/L(R1700L)-Ipilimumab (=rgNDV-NoThaBene-2b-Ipilimumab)
rgNDV-Mut HN(F277L)/M(G165W)/F(F117S)/F(F190L)/F(L289A)/L(V757I)/L(F1551S)/L(R1700L)-IL-12 (=rgNDV-NoThaBene-2b-IL-12)
rgNDV-Mut HN(F277L)/M(G165W)/F(F117S)/F(F190L)/F(L289A)/L(V757I)/L(F1551S)/L(R1700L)-NS1 (=rgNDV-NoThaBene-2b-NS1)
rgNDV-Mut HN(F277L)/M(G165W)/F(F117S)/F(F190L)/F(L289A)/L(V757I)/L(F1551S)/L(R1700L)-Apoptin (=rgNDV-NoThaBene-2b-Apoptin)
rgNDV-Mut HN(F277L)/M(G165W)/F(F117S)/F(F190L)/F(L289A)/L(V757I)/L(F1551S)/L(R1700L)-B18R (=rgNDV-NoThaBene-2b-B18R)
rgNDV-Mut HN(F277L)/M(G165W)/F(F117S)/F(F190L)/F(L289A)/L(V757I)/L(F1551S)/L(R1700L)-Theralizumab (=rgNDV-NoThaBene-2b-Theralizumab)
rgNDV-Mut HN(F277L)/M(G165W)/F(F117S)/F(F190L)/F(L289A)/L(V757I)/L(F1551S)/L(R1700L)-anti-CD40 (=rgNDV-NoThaBene-2b-anti-CD40)
rgNDV-Mut HN(F277L)/M(G165W)/F(F117S)/F(F190L)/F(L289A)/L(V757I)/L(F1551S)/L(R1700L)-anti-CD80 (=rgNDV-NoThaBene-2b-anti-CD80)
rgNDV-Mut HN(F277L)/M(G165W)/F(F117S)/F(F190L)/F(L289A)/L(V757I)/L(F1551S)/L(R1700L)-anti-CA 15-3 (=rgNDV-NoThaBene-2b-anti-CA 15-3)
rgNDV-Mut HN(F277L)/M(G165W)/F(F117S)/F(F190L)/F(L289A)/L(V757I)/L(F1551S)/L(R1700L)-anti CA 19-9 (=rgNDV-NoThaBene-2b-anti CA 19-9)
rgNDV-Mut HN(F277L)/M(G165W)/F(F117S)/F(F190L)/F(L289A)/L(V757I)/L(F1551S)/L(R1700L)-Sofituzumab (=rgNDV-NoThaBene-2b-Sofituzumab)
rgNDV-Mut HN(F277L)/M(G165W)/F(F117S)/F(F190L)/F(L289A)/L(V757I)/L(F1551S)/L(R1700L)-Cetuximab (=rgNDV-NoThaBene-2b-Cetuximab)
rgNDV-Mut HN(F277L)/M(G165W)/F(F117S)/F(F190L)/F(L289A)/L(V757I)/L(F1551S)/L(R1700L)-Trastuzumab (=rgNDV-NoThaBene-2b-Trastuzumab)
rgNDV-Mut HN(F277L)/M(G165W)/F(F117S)/F(F190L)/F(L289A)/L(V757I)/L(F1551S)/L(R1700L)-BIL=3s (=rgNDV-NoThaBene-2b-BIL=3s)
rgNDV-Mut HN(F277L)/M(G165W)/F(F117S)/F(F190L)/F(L289A)/L(V757I)/L(F1551S)/L(R1700L)-J591 (=rgNDV-NoThaBene-2b-J591)
rgNDV-Mut HN(F277L)/M(G165W)/F(F117S)/F(F190L)/F(L289A)/L(V757I)/L(F1551S)/L(R1700L)-Ramucircumab (=rgNDV-NoThaBene-2b-Ramu-circumab)
rgNDV-Mut HN(F277L)/M(G165W)/F(F117S)/F(F190L)/F(L289A)/L(V757I)/L(F1551S)/L(R1700L)-TRAIL (=rgNDV-NoThaBene-2b-TRAIL).

A NDV or rgNDV according to the present invention can be provided in a suitable cell or cell line, for example a HeLa cell line or in cancer cells, or an embryonated egg that is susceptible to a NDV infection.

In these infected cells the virus is grown to sufficient quantities, and preferably under sufficient conditions such that the virus is free from exogenous contamination, and the progeny virus particles are collected by suitable methods well known to those skilled in the art.

Even though the NDV strains as described above, for example the F3L3 strains, are particularly beneficial for expressing the foreign genes as mentioned above, also F2L3 strains of NDV can be advantageously used to express these foreign genes. Even more general, NDV strains having the mutation in the HN gene relating to the amino acid position 277 as specified above and having at least one mutation in the F gene concerning amino acid position 117 and/or amino acid position 190 as specified, but not the mutation at amino acid position 289, above can be beneficially used.

Thus, in a second aspect the present invention also encompasses the recombinant NDV strains comprising a viral genome comprising a nucleic acid comprising a nucleic acid sequence encoding at least one foreign gene. The at least one foreign gene being selected from the following “group B”:
A gene encoding an antibody capable of blocking checkpoint inhibition, such as an antibody directed against checkpoint inhibition protein PD-1 (programmed cell death protein 1, also known as CD279) or an antigen-binding part directed to protein PD-1 (anti-PD-1). They work by blocking a signal that prevents activated T cells from attacking the cancer upon expression in rNDV-expresser cells, thus allowing the immune system to clear the cancer. In one embodiment the antibody is Nivolumab, an antigen-binding part of Nivolumab, a variant of Nivolumab or a variant of an antigen-binding part of Nivolumab.
A gene encoding an antibody capable of blocking checkpoint inhibition, such as an antibody directed to the surface protein CTLA-4 or an antigen-binding part directed to protein CTLA-4 (anti-CTLA-4). In one embodiment the antibody is Ipilimumab, an antigen-binding part of Ipilimumab or a variant of Ipilimumab or a variant of an antigen-binding part of Ipilimumab.
A gene encoding a protein which improves the cellular immune response and improves the ability of T cells to enter tumor cells, or a part thereof which improves the cellular immune response and improves the ability of T cells to enter tumor cells. In one embodiment the protein is interleukin-12 (IL-12) or a part of interleukin-12 or a variant of interleukin-12 or a variant of a part of interleukin-12.
A gene encoding a protein with the ability to modulate the virus replication cycle, or a part thereof with the ability to modulate the virus replication cycle. In one embodiment the protein is the non-structural protein NS1 of influenza A virus or a part of the non-structural protein NS1 of influenza A virus or a variant of the non-structural protein NS1 of influenza A virus or a variant of a part of the non-structural protein NS1 of influenza A virus.
A gene encoding a protein with the ability to selectively induce apoptosis in human tumor cells, but not in normal human cells, or a part thereof with the ability to selectively induce apoptosis in human tumor cells, but not in normal human cells. In one embodiment the protein is Apoptin (VP3 from chicken anemia virus) or a part of Apoptin or a variant of Apoptin or a variant of a part of Apoptin.
A gene encoding a protein that reduces or inhibits IFN expression such as an IFN-beta receptor, or a part thereof that reduces or inhibits IFN expression such as an IFN-beta receptor. In one embodiment the protein is the viral protein B18R from vaccinia virus or a part of the viral protein B18R from vaccinia virus or a variant of the viral protein B18R from vaccinia virus or a variant of a part of the viral protein B18R from vaccinia virus. B18R is a homologue of the human IFN-β receptor. B18R acts as a decoy for IFN-β, thereby inhibiting activation by cell signaling of the antiviral host response in native cells, resulting in increased lytic activity in cancer cells.
A gene encoding the protein CD40 (cluster of differentiation 40), a part of CD40, a variant of CD40 or a variant of a part of CD40.
A gene encoding an antibody, preferably an agonistic antibody, directed to CD28 or an antigen-binding part directed to CD28 (anti-CD28). CD28 (cluster of differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins. In one embodiment the antibody is Theralizumab, an antigen-binding part of Theralizumab, a variant of Theralizumab or a variant of an antigen-binding part of Theralizumab.
A gene encoding an antibody, preferably an agonistic antibody, directed to CD40 or an antigen-binding part directed to CD40 (anti-CD40). CD40 (cluster of differentiation 40) is a costimulatory protein found on antigen-presenting cells and is required for their activation. Thus, the antibody or its antigen-binding part is used to activate an anti-tumor T cell response via activation of the antigen-presenting cells.
A gene encoding an antibody, preferably an agonistic antibody, directed to CD80 or an antigen-binding part directed to CD80 (anti-CD80). CD80 (cluster of differentiation 80) is a membrane protein which is the receptor for the proteins CD28 and CTLA-4, and which membrane protein is involved in the costimulatory signal essential for T-lymphocyte activation. Thus, the antibody or its antigen-binding part is used to activate T-lymphocytes.
A gene encoding an antibody directed to CA 15-3 or an antigen-binding part directed to CA 15-3 (anti-CA 15-3). CA 15-3 is also known as the human gene MUC1.
A gene encoding an antibody directed to CA 19-9 or an antigen-binding part directed to CA 19-9 (anti-CA 19-9). CA 19-9 is a muzin which corresponds to the sialylated Lewisa blood group antigen.
A gene encoding an antibody directed to CA 125 or an antigen-binding part directed to CA 125 (anti-CA 125). CA 125 is also known as the human gene MUC16. An antibody or its antigen-binding part targeting the MUC16 protein results in a G2/M-phase growth arrest and tumour cell apoptosis. In one embodiment the antibody is Sofituzumab, an antigen-binding part of Sofituzumab, a variant of Sofituzumab or a variant of an antigen-binding part of Sofituzumab.
A gene encoding an antibody, preferably an antagonistic antibody, directed to EGFR or an antigen-binding part directed to EGFR (anti-EGFR). The epidermal growth factor receptor (EGFR) is a transmembrane protein. Permanent activation of the receptor results in an uncontrolled cell proliferation and prevents controlled cell death (apoptosis), which contributes to the malignant transformation of cells. The antagonistic antibody is directed against the extracellular domain of the EGFR and leads to an inhibition of ligand binding. In one embodiment the antibody is Cetuximab, an antigen-binding part of Cetuximab, a variant of Cetuximab or a variant of an antigen-binding part of Cetuximab.
A gene encoding an antibody, preferably an antagonistic antibody, directed to HER2 or an antigen-binding part directed to HER2 (anti-HER2). The antibody or its antigen-binding part is targeting the extracellular part of the HER2 receptor, which is overexpressed in certain cancers, and is intended to interrupt the growth-promoting signals. In one embodiment the antibody is Trastuzumab, an antigen-binding part of Trastuzumab, a variant of Trastuzumab or a variant of an antigen-binding part of Trastuzumab.
A gene encoding an antibody directed to nfP2X₇ or an antigen-binding part directed to nfP2X₇ (anti-nfP2X₇). The non-functioning P2X₇ receptor is a form of the ATP-gated P2X₇ receptor in which the E200 epitope is exposed for antibody binding. The nfP2X₇ is able to form a Ca²⁺ channel but not an apoptotic pore and is found on every type of cancer cell but no normal cells. In one embodiment the antibody is BIL=3s, an antigen-binding part of BIL=3s, a variant of BIL=3s or a variant of an antigen-binding part of BIL=3 s.
A gene encoding an antibody directed to PSMA or an antigen-binding part directed to PSMA (anti-PSMA). The prostate specific membrane antigen (PSMA) is a cell surface peptidase highly expressed by malignant prostate epithelial cells and vascular endothelial cells of numerous solid tumor malignancies. In one embodiment the antibody is J591, an antigen-binding part of J591, a variant of J591 or a variant of an antigen-binding part of J591.
A gene encoding an antibody, preferably an antagonistic antibody, directed to VEGFR or an antigen-binding part directed to VEGFR (anti-VEGFR). The vascular endothelial growth factor (VEGF) is involved in angiogenesis. The natural ligands comprise VEGF-A, VEGF-C and VEGF-D. Antagonistic antibodies targeting VEGF lead to inhibition of VEGF-mediated tumor angiogenesis. In one embodiment the antibody is Ramucirumab, an antigen-binding part of Ramucirumab, a variant of Ramucirumab or a variant of an antigen-binding part of Ramucirumab.
A gene encoding a protein that induces the process of apoptosis by binding to a death receptor, such as death receptor DR4 and/or death receptor DR5, or a part thereof that induces the process of apoptosis by binding to a death receptor, such as death receptor DR4 and/or death receptor DR5. In one embodiment the protein is TRAIL, a part of TRAIL, a variant of TRAIL or a variant of a part of TRAIL.
Any combination of these genes, parts or variants of these genes or parts thereof.

In the following all these genes encoding the respective antibodies, antigen-binding parts, proteins or parts thereof as well as any of the preferably mentioned antibodies, antigen-bindings parts, proteins, parts or variants thereof, are referred to in the following as “the foreign genes according to group B”. This group B also encompasses any combination of these genes, parts or variants of these genes or parts thereof.

The respective proteins or antibodies or parts thereof are also referred to in the following as “the foreign gene products according to group B”. This group also encompasses any combination of these gene products, parts or variants thereof.

In another embodiment of the present invention a recombinant NDV comprises in its viral genome at least two foreign genes or parts or variants thereof, which foreign genes or parts or variants are selected from the above mentioned foreign genes according to group B.

Preferred embodiments of a nucleic acid comprising a nucleic acid sequence encoding at least one foreign gene according to group B are given in SEQ ID No. 15 to 30 of the sequence listing. The transgenic construct can also be a variant or part of any of the sequences according to SEQ ID No. 15 to 30 of the sequence listing.

In addition to comprising a transgenic construct comprising a nucleic acid sequence encoding at least one foreign gene according to group B, the viral genome of the recombinant NDV strains according to the present invention further comprise a nucleic acid comprising a nucleic acid sequence (HN gene) encoding a HN protein having a change in the amino acid sequence of the HN protein and providing the NDV with an increased replication capability in a cancer cell, preferably in a human cancer cell, as compared to an otherwise identical NDV not having the said mutation in the HN gene.

Preferably the mutation in the HN gene results in an unexpected beneficially increased replication capability of the recombinant NDV strain in cancer cells, preferably in human cancer cells, and more preferably results in a replication of said oncolytic NDV in a human cancer cell which is at least 2-fold higher, more preferably 2- to 10-fold higher, still more preferably 5- to 10-fold higher than an NDV parent strain not having said mutation in its viral HN gene. Preferably, said parent strain comprises or is an oncolytic NDV already having an advantageous safety and efficacy profile in humans, such as the NDV strain MTH-68/H.

According to a preferred embodiment the recombinant NDV strain comprises a mutated HN gene encoding a hemagglutinin-neuramidase (HN protein) with an amino acid substitution at position 277 to an amino acid with a hydrophobic side chain other than phenylalanine. As it relates to preferred and more preferred embodiments of the substitution at amino acid position 277 of the HN protein reference to above disclosure with regard to the novel NDV strains is made. The same preferred and more preferred embodiments/amino acids apply for these recombinant NDV strains according to the second aspect of the present invention.

The oncolytic NDV strain having this mutation in the HN gene and carrying at least one of the foreign genes according to group B can be derived from NDV strain MTH-68/H.

According to the present invention the recombinant and mutated NDV strains comprise in addition to the above described mutated HN gene, a nucleic acid sequence (F gene) encoding a F protein comprising at least one mutation at amino acid position 117 and/or amino acid position 190. The combination of both mutations at amino acid position 117 and 190 of the F gene is particularly preferred. As it relates to preferred and more preferred embodiments of the substitutions at amino acid position 117 and amino acid position 190 of the F gene reference to above disclosure with regard to the novel NDV strains is made. The same preferred and more preferred embodiments/amino acids apply for these recombinant NDV strains according to the second aspect of the present invention. The recombinant NDV strain can be a NDV strain derived from NDV strain MTH-68/H having the mutation in the HN gene product or HN protein at position 277 and having at least one mutation in the F gene at amino acid position 117 and/or 190. A strain comprising the mutation at amino acid position 117 is also referred to as NDV-Mut HN(F277L)/F(F117S). That is, the same nomenclature as explained above is used. The strain can also be NDV-Mut HN(F277L)/F(F190L) or NDV-Mut HN(F277L)/F(F117S)/F(F190L). The transgenic construct comprising the foreign gene or part or respective variant thereof carried by these strains is referred to as Nivolumab, Ipilimumab, IL-12, NS1, Apoptin, B18R, CD40, Theralizumab, anti-CD40, anti-CD80, anti-CA 15-3, anti CA 19-9, Sofituzumab, Cetuximab, Trastuzumab, BIL=3s, J591, Ramucircumab or TRAIL in this shortcut for the strain. Within these shortcuts the meaning of the respective foreign gene is as explained above.

The following NDV strains are provided by the present invention in a preferred embodiment:
NDV-Mut HN(F277L)/F(F117S)/F(F190L)-Nivolumab
NDV-Mut HN(F277L)/F(F117S)/F(F190L)-Ipilimumab
NDV-Mut HN(F277L)/F(F117S)/F(F190L)-IL-12
NDV-Mut HN(F277L)/F(F117S)/F(F190L)-NS1
NDV-Mut HN(F277L)/F(F117S)/F(F190L)-Apoptin
NDV-Mut HN(F277L)/F(F117S)/F(F190L)-B18R
NDV-Mut HN(F277L)/F(F117S)/F(F190L)-CD40
NDV-Mut HN(F277L)/F(F117S)/F(F190L)-Theralizumab
NDV-Mut HN(F277L)/F(F117S)/F(F190L)-anti-CD40
NDV-Mut HN(F277L)/F(F117S)/F(F190L)-anti-CD80
NDV-Mut HN(F277L)/F(F117S)/F(F190L)-anti-CA 15-3
NDV-Mut HN(F277L)/F(F117S)/F(F190L)-anti-CA 19-9
NDV-Mut HN(F277L)/F(F117S)/F(F190L)-Sofituzumab
NDV-Mut HN(F277L)/F(F117S)/F(F190L)-Trastuzumab
NDV-Mut HN(F277L)/F(F117S)/F(F190L)-BIL=3s
NDV-Mut HN(F277L)/F(F117S)/F(F190L)-J591
NDV-Mut HN(F277L)/F(F117S)/F(F190L)-Ramucircumab
NDV-Mut HN(F277L)/F(F117S)/F(F190L)-TRAIL.

It has been surprisingly shown that the at least one mutation, preferably both mutations (also referred to as “F2” herein), in the F gene at position 117 and 190 of the amino acid sequence of the resulting F protein beneficially improves the oncolytic potential of the respective NDV strains by improving the safety profile of the viruses in humans. The respective amino acid substitutions are as specified supra.

With regard to the viral safety profile, this in particular means that the intracerebral pathogenicity index (ICPI) is reduced by these mutations in the F gene, preferably F2, The ICPI value indicates the the pathogenicity of a Newcastle disease virus. Preferably, the ICPI is reduced by at least 10%, more preferably by at least 20% and still more preferred by at least 30%, as compared to the identically determined ICPI value of a NDV strain not having the respective mutation(s) in the F gene. The ICPI can be determined according to the method set forth in World Organization for Animal Health (OIE) 2009, Chapter 2.3.14. Newcastle disease, pp. 576-589, in: Manual of Diagnostic Tests and Vaccines for Terrestrial Animals 2009, World Organization for Animal Health (OIE), Paris.

The recombinant and mutated NDV strains according to the second aspect of the present invention can comprise in addition to the above described mutation in the HN gene, and the at least one mutation in the F gene at amino acid position 117 and/or 190, a mutation in the M gene and the thus encoded matrix protein, preferably at amino acid position 165. Again, as it relates to preferred and more preferred embodiments of the substitution at amino acid position 165 of the M gene reference to above disclosure with regard to the novel NDV strains is made. The same preferred and more preferred embodiments/amino acids apply for these recombinant NDV strains according to the second aspect of the present invention.

According to one embodiment of the present invention a recombinant NDV strain comprises as a foreign gene at least one gene selected from the foreign genes according to group B, and has a mutation in the HN gene, preferably resulting in a substitution of the amino acid phenylalanine (F) to leucine (L) at position 277 of the HN gene product, a mutation in the M gene, preferably resulting in a substitution of the amino acid glycine (G) to the amino acid tryptophane (W) at position 165 of the M gene product, and at least one mutation in the F gene, preferably resulting in a substitution of the amino acid phenylalanine (F) to the amino acid serine (S) at position 117 of the F gene product, and/or resulting in a substitution of the amino acid phenylalanine (F) to the amino acid leucine (L) at position 190 of the F gene product. The combination of both mutations at amino acid position 117 and 190 of the F gene is particularly preferred. As explained supra, other substitutions at these amino acid positions are also comprised within the present invention.

In a preferred embodiment of the present invention the recombinant and mutated NDV strains comprise in addition to the above described mutation in the HN gene and the at least one mutation in the F gene at amino acid positions 117 and/or 190 as specified above, at least one mutation in the L gene and the thus encoded RNA-dependent RNA polymerase protein, also referred to as L protein or L gene product herein. Still more preferred the mutated L gene encodes a RNA-dependent RNA polymerase protein with an amino acid substitution at position 757 and/or position 1551 and/or 1700. Preferably the NDV comprises a mutated L gene and the encoded L protein with the amino acid substitution at position 757, position 1551 and position 1700. Again, as it relates to preferred and more preferred embodiments of the substitutions at amino acid positions 757, 1551 and 1700 of the L gene reference to above disclosure with regard to the novel NDV strains is made. The same preferred and more preferred embodiments/amino acids apply for these recombinant NDV strains according to the second aspect of the present invention. These recombinant NDV strains may in addition also comprise the above described mutation in the M gene.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, having two mutations in the F gene, wherein the amino acid phenylalanine (F) is substituted to the amino acid serine (W) at position 117 and the amino acid phenylalanine (F) is substituted to the amino acid leucine (L) at position 190 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/L(V757I)/L(F1551S)/L(R1700L) or MutHu2 or NDV-NoThaBene-2. The foreign gene or part or variant thereof carried by this strain is referred to as Nivolumab, Ipilimumab, IL-12, NS1, Apoptin, B18R, CD40, Theralizumab, anti-CD40, anti-CD80, anti-CA 15-3, anti CA 19-9, Sofituzumab, Cetuximab, Trastuzumab, BIL=3s, J591, Ramucircumab or TRAIL. Within these shortcuts for the recombinant NDV strains a reference to the respective foreign gene is intended to have the meaning as already defined above. The following NDV strains are provided by the present invention in a particularly preferred embodiment:
NDV-Mut HN(F277L)/M(G165W)/F(F117S)/F(F190L)/L(V757I)/L(F1551S)/L(R1700L)-Nivolumab,
NDV-Mut HN(F277L)/M(G165W)/F(F117S)/F(F190L)/L(V757I)/L(F1551S)/L(R1700L)-Ipilimumab,
NDV-Mut HN(F277L)/M(G165W)/F(F117S)/F(F190L)/L(V757I)/L(F1551S)/L(R1700L)-IL-12,
NDV-Mut HN(F277L)/M(G165W)/F(F117S)/F(F190L)/L(V757I)/L(F1551S)/L(R1700L)-NS1,
NDV-Mut HN(F277L)/M(G165W)/F(F117S)/F(F190L)/L(V757I)/L(F1551S)/L(R1700L)-Apoptin,
NDV-Mut HN(F277L)/M(G165W)/F(F117S)/F(F190L)/L(V757I)/L(F1551S)/L(R1700L)-B18R,
NDV-Mut HN(F277L)/M(G165W)/F(F117S)/F(F190L)/L(V757I)/L(F1551S)/L(R1700L)-CD40,
NDV-Mut HN(F277L)/M(G165W)/F(F117S)/F(F190L)/L(V757I)/L(F1551S)/L(R1700L)-Theralizumab,
NDV-Mut HN(F277L)/M(G165W)/F(F117S)/F(F190L)/L(V757I)/L(F1551S)/L(R1700L)-anti-CD40,
NDV-Mut HN(F277L)/M(G165W)/F(F117S)/F(F190L)/L(V757I)/L(F1551S)/L(R1700L)-anti-CD80,
NDV-Mut HN(F277L)/M(G165W)/F(F117S)/F(F190L)/L(V757I)/L(F1551S)/L(R1700L)-anti-CA 15-3,
NDV-Mut HN(F277L)/M(G165W)/F(F117S)/F(F190L)/L(V757I)/L(F1551S)/L(R1700L)-anti CA 19-9,
NDV-Mut HN(F277L)/M(G165W)/F(F117S)/F(F190L)/L(V757I)/L(F1551S)/L(R1700L)-Sofituzumab,
NDV-Mut HN(F277L)/M(G165W)/F(F117S)/F(F190L)/L(V757I)/L(F1551S)/L(R1700L)-Cetuximab,
NDV-Mut HN(F277L)/M(G165W)/F(F117S)/F(F190L)/L(V757I)/L(F1551S)/L(R1700L)-Trastuzumab,
NDV-Mut HN(F277L)/M(G165W)/F(F117S)/F(F190L)/L(V757I)/L(F1551S)/L(R1700L)-BIL=3s,
NDV-Mut HN(F277L)/M(G165W)/F(F117S)/F(F190L)/L(V757I)/L(F1551S)/L(R1700L)-J591,
NDV-Mut HN(F277L)/M(G165W)/F(F117S)/F(F190L)/L(V757I)/L(F1551S)/L(R1700L)-Ramucircumab,
NDV-Mut HN(F277L)/M(G165W)/F(F117S)/F(F190L)/L(V757I)/L(F1551S)/L(R1700L)-TRAIL.

The two mutations in the F gene at position 117 and 190 of the amino acid sequence of the resulting F protein and the three mutations in the L gene at position 757, 1551 and 1700 of the amino acid sequence of the resulting L protein, either alone or in combination, have been shown to beneficially improve the oncolytic potential of the respective NDV strains by improving the safety profile of the viruses in humans. The combinations include any combination of the referenced mutations in the F gene and L gene, and the F protein and L protein, respectively. That is any combination from two to five mutations. Particularly preferred is a combination of both mutations at amino acid position 117 and 190 of the F gene (also referred to as “F2” herein) and a combination of all five mutations, that is mutations at positions 117 and 190 of the F gene product and at positions 757, 1551 and 1700 of the L gene product (also referred to as “F2L3” herein). The respective amino acid substitutions are as specified supra. The F2 and F2L3 mutations result in a reduced intracerebral pathogenicity index (ICPI). Preferably, the ICPI is reduced by at least 10%, more preferably by at least 20% and still more preferred by at least 30%, as compared to the identically determined ICPI value of a NDV strain not having the respective mutations in the F gene and the L gene.

According to another embodiment of the present invention a recombinant NDV according to the present invention may also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product.

Alternatively or in addition a recombinant NDV according to the present invention may comprise a mutation in the RNA-editing sequence of the P gene. More preferably, the mutation abolishes and/or decreases the expression of the V protein.

Table 1a shown above gives a list of mutations for obtaining NDV variants with increased titer production and/or good or improved safety profile according to the present invention. The mutation in the HN gene and at least one mutation in the F gene at amino acid position 117 or 190 are comprised in all these NDV strains according to the present invention, while the mentioned mutations in the M gene and L gene may or may not be present. As it relates to the third mutation in the F gene at amino acid position 289, this mutation is not mandatory for the recombinant NDV strains according to the second aspect of the present invention.

Table 1e shows particularly preferred NDV variants with increased titer production and/or improved safety profile in line with the second aspect of the present invention, and the parent NDV strain MTH-68/H. “+” means that at the mentioned amino acid position the original (first mentioned) amino acid is substituted by the last mentioned amino acid, while “−” means that there is no amino acid substitution at the mentioned position, i.e. the first mentioned amino acid is present. For example and with regard to the HN gene “+” means that at amino acid position 277 phenylalanine (F) is substituted to leucine (L).

TABLE 1e

| | Relevant | | | | | | | | | |
|---|---|---|---|---|---|---|---|---|---|---|
| | amino | MTH- | | | | | | | | |
| Gene | acid | 68/H | | | | | | | | |
| HN | F277L | − | + | + | + | + | + | + | + | + |
| M | G165W | − | − | + | − | + | − | + | − | + |
| F | F117S | − | + | + | + | + | + | + | + | + |
| F | F190L | − | + | + | + | + | + | + | + | + |
| F | L289A | − | − | − | + | + | − | − | + | + |
| L | V757I | − | − | − | − | − | + | + | + | + |
| L | F1551S | − | − | − | − | − | + | + | + | + |
| L | R1700L | − | − | − | − | − | + | + | + | + |
| L | Y1717H | − | − | − | − | − | − | − | + | + |
| L | E1910K | − | − | − | − | − | − | − | + | + |

Table 1f shows all mutations in the F gene encompassed by the second aspect of the present invention. “+” means that at the mentioned amino acid position the original (first mentioned) amino acid is substituted by the last mentioned amino acid, while “−” means that there is no amino acid substitution at the mentioned position, i.e. the first mentioned amino acid is present. According to the second aspect of the present invention at least one mutation at amino acid position 117 or 190 is mandatory. The combination of both mutations is particularly preferred.

TABLE 1f

| | Relevant | | | | | | |
|---|---|---|---|---|---|---|---|
| | amino | | | | | | |
| Gene | acid | 1 | 1 | 2 | 2 | 2 | 3 |
| F | F117S | + | − | + | + | − | + |
| F | F190L | − | + | + | − | + | + |
| F | L289A | − | − | − | + | + | + |

As it relates to preferred mutations in the L gene reference to Table 1d shown above is made.

In a preferred embodiment the NDV and the recombinant NDV resulting thereof is encoded by and/or comprises at least one of the nucleic acids according to SEQ ID No. 1 or any one of SEQ ID No. 36 to 38 or parts thereof. According to another preferred the recombinant NDV strains of the present invention have a sequence identity of at least 75%, particularly of at least 90%, more particularly of at least 95% to any one of SEQ ID No. 1 or SEQ ID No. 36 to 38. The sequences given by these SEQ ID numbers are as follows:
SEQ ID No. 1: Nucleic acid (cDNA) sequence of the Newcastle disease virus (NDV) strain MTH-68/H genome;
SEQ ID No. 36: Nucleic acid (cDNA) sequence of Newcastle disease virus strain Mut HN(F277L)/M(G165W)/F(F117S)/F(F190L)/L(V757I)/L(F1 551S)/L(R1700L) genome having a mutation in the HN gene, a mutation in the M gene, two mutations in the F gene (F2) and three mutations in the L gene (L3) (=NDV-NoThaBene-2 genome).
SEQ ID No. 37: Nucleic acid (cDNA) sequence of Newcastle disease virus strain according to SEQ ID No.35, but comprising in addition a 12 bp insert containing a AfeI restriction site according to SEQ ID no. 4 between nucleotide positions 3134 and 3135 of SEQ ID No.35.
SEQ ID No. 38: Nucleic acid (cDNA) sequence of Newcastle disease virus strain Mut HN(F277L)/M(G165W)/F(F117S)/F(F190L)/L(V757I)/L(F1 551S)/L(R1700L)-Lirilumab genome created by reverse genetics as disclosed herein, wherein the nucleic acid comprises a mutation in the HN gene, a mutation in the M gene, two mutations in the F gene (F2) and three mutations in the L gene (L3), and additionally encodes Lirilumab.

The SEQ ID No. 38 of the sequence listing shows by way of example a transgenic NDV strain according to the present invention. Shown is a nucleic acid (cDNA) sequence of Newcastle disease virus strain Mut HN(F277L)/M(G165W)/F(F117S)/F(F190L)/L(V757I)/L(F1551S)/L(R1700L)-Lirilumab genome created by reverse genetics as disclosed herein, wherein the nucleic acid comprises a mutation in the HN gene, a mutation in the M gene, two mutations in the F gene (F2) and three mutations in the L gene (L3), and additionally encodes Lirilumab.

The transgenic construct comprising the nucleic acid sequence encoding Lirilumab is in this example inserted in the 12 nucleotide long sequence according to SEQ ID No. 4 of the sequence listing in the AfeI-site.

Instead of the nucleic acid sequence comprising a nucleic acid sequence encoding anti-KIR, preferably Lirilumab, any other nucleic acid sequence according to any one of SEQ ID No. 5 to 25 and SEQ ID No. 27 to 30 can be inserted to obtain a preferred transgenic NDV strain according to the present invention.

In a preferred embodiment of the present invention the viral HN gene product has an amino acid sequence as identified in SEQ ID No. 32, and/or the viral M gene product preferably has an amino acid sequence as identified in SEQ ID No. 34. In addition it is preferred that a NDV strain according to the present invention comprises beside the viral HN gene product having an amino acid sequence as identified in SEQ ID No. 32, and/or the viral M gene product preferably having an amino acid sequence as identified in SEQ ID No. 34, a viral F gene product having an amino acid sequence as identified in SEQ ID No. 39 and/or a viral L gene product having an amino acid sequence as identified in SEQ ID No. 35. Also encompassed by the present invention are NDV strains comprising variants of the gene products according to SEQ ID No. 32, 34, 35 or 39.

The present invention also provides a recombinant nucleic acid comprising the nucleic acid sequence of the recombinant NDV strains according to the second aspect of the present invention. The nucleic acid comprises a transgenic construct. The transgenic construct comprises a nucleic acid sequence encoding at least one foreign gene or transgene. The foreign gene can encode a gene product according to group B. The transgenic construct comprised in the nucleic acid can also be a combination of two or more of the foreign genes according to group B. The transgenic construct can be or comprise a nucleic acid sequence according to any one of SEQ ID No. 15 to 30 of the sequence listing or a part or variant thereof.

In addition to the transgenic construct a nucleic acid according to the present invention comprises a nucleic acid sequence (HN gene) encoding a mutated HN protein. The encoded HN protein is preferably mutated at amino acid position 277. As it relates to preferred and more preferred embodiments of the substitution at amino acid position 277 of the HN gene reference to above disclosure with regard to the novel NDV strains according to the first aspect of the present invention is made. The same preferred and more preferred embodiments/amino acids apply for the recombinant NDV strains according to the second aspect of the present invention.

In addition a nucleic acid according to the present invention comprises a nucleic acid sequence (F gene) having at least one mutation. Preferably the mutated F gene encodes a fusion protein with an amino acid substitution at position 117 and/or position 190. As it relates to preferred and more preferred embodiments of the substitutions at amino acid positions 117 and 190 of the F gene reference to above disclosure with regard to the novel NDV strains according to the first aspect of the present invention is made. The same preferred and more preferred embodiments apply for the recombinant NDV strains according to the second aspect of the present invention.

In a particular preferred embodiment the nucleic acid is derived from the NDV strain MTH-68/H.

The nucleic acid sequence encoding the recombinant NDV can also comprise a nucleic acid sequence (M gene) having a mutation. Preferably the mutated M gene encodes a matrix protein with an amino acid substitution at position 165 to an amino acid with an aromatic side chain. As it relates to preferred and more preferred embodiments of the substitution at amino acid position 165 of the M gene reference to above disclosure with regard to the novel NDV strains according to the first aspect of the present invention is made. The same preferred and more preferred embodiments/amino acids apply for the recombinant NDV strains according to the second aspect of the present invention.

In a particular preferred embodiment the nucleic acid is derived from the NDV strain MTH-68/H and comprises a mutation in the HN gene resulting in an amino acid with a hydrophobic side chain other than phenylalanine at amino acid position 277, a mutation in the M gene resulting in a matrix protein with an amino acid substitution at position 165 to an amino acid with an aromatic side chain, and at least one mutation in the F gene resulting in a fusion protein with an amino acid substitution at position 117 to an amino acid with a hydroxylated side chain and/or an amino acid substitution at position 190 to an amino acid with an aliphatic side chain. Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), and the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L). The combination of both mutations at amino acid position 117 (F^{F117S}) and 190 (F^{F190L}) of the F gene is particularly preferred.

According to a further preferred embodiment of the present invention the sequence encoding the recombinant NDV comprises, in addition to the above described mutation in the HN gene, particularly the mutation resulting in an amino acid substitution at position 277 of the HN gene product as specified supra, and in addition to the above described at least one mutation in the F gene, particularly the mutation resulting in an amino acid substitution at position 117 (F^{F117S}) and/or at position 190 (F^{F190L}) of the F gene product as specified supra, optionally in combination with the above described mutation on the M gene, particularly the mutation resulting in an amino acid exchange at position 165 of the M gene product as specified supra, also comprises at least one mutation in the L gene. Preferably the mutated L gene encodes for a L protein with at least one amino acid substitution at amino acid position 757, 1551 and/or 1700. As it relates to preferred and more preferred embodiments of the substitutions at amino acid positions 757, 1551 and/or 1700 of the L gene reference to above disclosure with regard to the novel NDV strains according to the first aspect of the present invention is made. The same preferred and more preferred embodiments apply for the recombinant NDV strains according to the second aspect of the present invention. The mutations at amino acid positions 757, 1551 and 1700 of the L gene may be present alone or in each possible combination, i.e. position 757 and/or position 1551 and/or position 1700. The combination of all three mutations in the L gene (L3) is particularly preferred. In one embodiment of the present invention the at least one mutation in the L gene, preferably all three mutation at the respective amino acid positions 757, 1551 and 1700, is/are comprised in combination with the mutation in the HN gene as specified supra, the one or two mutations in the F gene as specified supra, and optionally, also in combination with the mutation in the M gene as specified supra.

In a particular preferred embodiment the nucleic acid is derived from the NDV strain MTH-68/H and comprises a mutation in the HN gene resulting in an amino acid with a hydrophobic side chain other than phenylalanine at position 277 of the respective amino acid sequence, a mutation in the M gene resulting in a matrix protein with an amino acid substitution at position 165 to an amino acid with an aromatic side chain, at least one mutation of the F gene resulting in a fusion protein with an amino acid substitution at position 117 to an amino acid with a hydroxylated side chain and/or an amino acid substitution at position 190 to an amino acid with an aliphatic side chain, and at least one mutation in the L gene resulting in a L protein with an amino acid substitution at position 757 to an amino acid with an aliphatic side chain other than valine (V), and/or an amino acid substitution at position 1551 to an amino acid with a hydroxylated side chain, and/or an amino acid substitution at position 1700 to an amino acid with an aliphatic side chain. More preferred, the mutated L gene results in a L protein comprising the three amino acid substitutions at positions 757, 1551 and 1700. Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L), and the mutated L gene encodes for a L protein in which the amino acid valine (V) at position 757 is substituted to the amino acid isoleucine (I), and/or the amino acid phenylalanine (F) at position 1551 is substituted to the amino acid serine (S), and/or the amino acid arginine (R) at position 1700 is substituted to the amino acid leucine (L). Still more preferred the L protein comprises all three mutations at positions 757, 1551 and 1700.

Also within the scope of the present invention are those nucleic acids comprising, in addition to the mutation in the HN gene and to the at least one mutation in the F gene at amino acid position 117 or 190, preferably in combination with one or more mutation in the M gene, and the L gene, as specified supra, at least one mutation in the L gene resulting in an amino acid substitution at position 1717 and/or at position 1910 of the L protein, and/or a mutation in the P gene as specified supra.

In one embodiment of the present invention the nucleic acid consists of or comprises at least one of the nucleic acids according to SEQ ID No. 36 or 37 or 38 or parts thereof. In another preferred embodiment the nucleic acid of the present invention has a sequence identity of at least 75%, particularly of at least 90%, more particularly of at least 95% to any of the sequences according SEQ ID No. 36 or 37 or 38.

In one embodiment of the present invention the nucleic acid is derived from a recombinant NDV as described supra.

As already mentioned above with regard to the first aspect of the present invention, a person skilled in the art knows how to use the disclosed sequences to produce synthetic DNAs (e.g. vectors, in particular in rg-NDV vectors), and how to use the synthetic DNAs to reconstruct and express the viral genome. Thus, the skilled artisan is enabled to produce virus particles by using the information disclosed herein. Reference is made to the ‘reverse genetics’ method as explained above. The difference with regard to the first aspect of the invention is that the amino acid substitution at position 289 of the F protein is not mandatory and that the transgenic construct is according to group B.

Therefore, the present invention also provides a method (herein also called ‘reverse genetics’) for providing cloned full-length cDNA of the recombinant NDV strains according to the second aspect of the present invention, and, in a further embodiment, the invention also provides infectious virus particles obtained from said full-length cDNA of the recombinant NDV strains according to the second aspect of the present invention. These recombinant NDV strains have an unexpected beneficial replication capability in cell cultures, eggs or animals that are used to propagate the virus for pharmaceutical purposes, and have good or even an improved viral safety profile, giving production advantages to such improved rgNDV strains. Furthermore, the higher replication rate is also particularly useful to obtain the desired oncolytic effects in vivo in that more virus particles will be produced for subsequent rounds of infection of cancer cells that were missed in the first round of infection. What is more, higher levels (yields) of the foreign gene or the foreign genes encoded by the transgenic construct will be achieved for providing the desired enhanced oncolytic effects in vivo. Thus, by modifying the HN protein within a transgene-expressing NDV higher yields of the transgenic construct protein can be obtained, which is very useful in anti-tumor immunotherapy. By modifying the F protein, and preferably also the L protein, as specified supra, also the viral safety can be improved.

The invention also provides an isolated, recombinant nucleic acid encoding a rgNDV obtainable by a method for preparing an rgNDV having improved replication in a cancer cell in comparison to that of a parent NDV, as described supra.

In one preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (a F277L mutation) allowing replication of said rgNDV in a human cancer cell to a higher level than replication of an otherwise identical rgNDV having the amino acid phenylalanine (F) at position 277 in the HN gene product, and with at least one mutation in the F gene resulting in a change of the amino acid phenylalanine (F) at amino acid position 117 of the fusion protein into serine (S), and/or resulting in a change of the amino acid phenylalanine (F) at amino acid position 190 of the fusion protein into leucine (L), said at least one mutation providing a decreased ICPI value than the ICPI of an otherwise identical rgNDV strain not having the at least one mutation in the F gene. These mutations are provided in combination with a transgenic construct encoding any of the foreign genes according to group B. The rgNDV may further comprise at least one or more mutation(s) selected from a mutation in the M gene, preferably G165W, a mutation at amino acid position 757, 1551, 1700, 1717 and/or 1910 in the L gene, preferably V757I, F1551S, R1700L, Y1717H and/or E1910K, and a mutation in the P gene, preferably the mutation in the P gene abolishes and/or decreases the expression of the V protein.

According to a particularly preferred embodiment the nucleic acid used to engineer a recombinant NDV genome is derived from the NDV strain MTH-68/H. Thus the present invention provides in one embodiment nucleic acids, preferably obtainable by the above specified method, which are or comprise
rgNDV-Mut HN(F277L)/F(F117S)/F(F190L)-Nivolumab,
rgNDV-Mut HN(F277L)/F(F117S)/F(F190L)-Ipilimumab,
rgNDV-Mut HN(F277L)/F(F117S)/F(F190L)-IL-12,
rgNDV-Mut HN(F277L)/F(F117S)/F(F190L)-NS1,
rgNDV-Mut HN(F277L)/F(F117S)/F(F190L)-Apoptin,
rgNDV-Mut HN(F277L)/F(F117S)/F(F190L)-B18R,
rgNDV-Mut HN(F277L)/F(F117S)/F(F190L)-CD40,
rgNDV-Mut HN(F277L)/F(F117S)/F(F190L)-Theralizumab,
rgNDV-Mut HN(F277L)/F(F117S)/F(F190L)-anti-CD40,
rgNDV-Mut HN(F277L)/F(F117S)/F(F190L)-anti-CD80,
rgNDV-Mut HN(F277L)/F(F117S)/F(F190L)-anti-CA 15-3,
rgNDV-Mut HN(F277L)/F(F117S)/F(F190L)-anti CA 19-9,
rgNDV-Mut HN(F277L)/F(F117S)/F(F190L)-Sofituzumab,
rgNDV-Mut HN(F277L)/F(F117S)/F(F190L)-Cetuximab,
rgNDV-Mut HN(F277L)/F(F117 S)/F(F190L)-Trastuzumab,
rgNDV-Mut HN(F277L)/F(F117S)/F(F190L)-BIL=3s,
rgNDV-Mut HN(F277L)/F(F117S)/F(F190L)-J591,
rgNDV-Mut HN(F277L)/F(F117S)/F(F190L)-Ramucircumab,
rgNDV-Mut HN(F277L)/F(F117S)/F(F190L)-TRAIL.

In another embodiment the present invention provides nucleic acids, preferably obtainable by the above specified method, which are or comprise
rgNDV-Mut HN(F277L)/M(G165W)/F(F117S)/F(F190L)/L(V757I)/L(F1551S)/L(R1700L)-Nivolumab (=rgNDV-NoThaBene-2-Nivolumab,
rgNDV-Mut HN(F277L)/M(G165W)/F(F117S)/F(F190L)/L(V757I)/L(F1551S)/L(R1700L)-Ipilimumab (=rgNDV-NoThaBene-2-Ipilimumab,
rgNDV-Mut HN(F277L)/M(G165W)/F(F117S)/F(F190L)/L(V757I)/L(F1551S)/L(R1700L)-IL-12 (=rgNDV-NoThaBene-2-IL-12,
rgNDV-Mut HN(F277L)/M(G165W)/F(F117S)/F(F190L)/L(V757I)/L(F1551S)/L(R1700L)-NS1 (=rgNDV-NoThaBene-2-NS1,
rgNDV-Mut HN(F277L)/M(G165W)/F(F117S)/F(F190L)/L(V757I)/L(F1551S)/L(R1700L)-Apoptin (=rgNDV-NoThaBene-2-Apoptin,
rgNDV-Mut HN(F277L)/M(G165W)/F(F117S)/F(F190L)/L(V757I)/L(F1551S)/L(R1700L)-B18R (=rgNDV-NoThaBene-2-B18R,
rgNDV-Mut HN(F277L)/M(G165W)/F(F117S)/F(F190L)/L(V757I)/L(F1551S)/L(R1700L)-CD40 (=rgNDV-NoThaBene-2-CD40,
rgNDV-Mut HN(F277L)/M(G165W)/F(F117S)/F(F190L)/L(V757I)/L(F1551S)/L(R1700L)-Theralizumab (=rgNDV-NoThaBene-2-Theralizumab,
rgNDV-Mut HN(F277L)/M(G165W)/F(F117S)/F(F190L)/L(V757I)/L(F1551S)/L(R1700L)-anti-CD40 (=rgNDV-NoThaBene-2-anti-CD40),
rgNDV-Mut HN(F277L)/M(G165W)/F(F117S)/F(F190L)/L(V757I)/L(F1551S)/L(R1700L)-anti-CD80 (=rgNDV-NoThaBene-2-anti-CD80),
rgNDV-Mut HN(F277L)/M(G165W)/F(F117S)/F(F190L)/L(V757I)/L(F1551S)/L(R1700L)-anti-CA 15-3 (=rgNDV-NoThaBene-2-anti-CA 15-3),
rgNDV-Mut HN(F277L)/M(G165W)/F(F117S)/F(F190L)/L(V757I)/L(F1551S)/L(R1700L)-anti CA 19-9 (=rgNDV-NoThaBene-2-anti CA 19-9),
rgNDV-Mut HN(F277L)/M(G165W)/F(F117S)/F(F190L)/L(V757I)/L(F1551S)/L(R1700L)-Sofituzumab (=rgNDV-NoThaBene-2-Sofituzumab),
rgNDV-Mut HN(F277L)/M(G165W)/F(F117S)/F(F190L)/L(V757I)/L(F1551S)/L(R1700L)-Cetuximab (=rgNDV-NoThaBene-2-Cetuximab),
rgNDV-Mut HN(F277L)/M(G165W)/F(F117S)/F(F190L)/L(V757I)/L(F1551S)/L(R1700L)-Trastuzumab (=rgNDV-NoThaBene-2-Trastuzumab),
rgNDV-Mut HN(F277L)/M(G165W)/F(F117S)/F(F190L)/L(V757I)/L(F1551S)/L(R1700L)-BIL=3s (=rgNDV-NoThaBene-2-BIL=3s),
rgNDV-Mut HN(F277L)/M(G165W)/F(F117S)/F(F190L)/L(V757I)/L(F1551S)/L(R1700L)-J591 (=rgNDV-NoThaBene-2-J591),
rgNDV-Mut HN(F277L)/M(G165W)/F(F117S)/F(F190L)/L(V757I)/L(F1551S)/L(R1700L)-Ramucircumab (=rgNDV-NoThaBene-2-Ramu-circumab),
rgNDV-Mut HN(F277L)/M(G165W)/F(F117S)/F(F190L)/L(V757I)/L(F1551S)/L(R1700L)-TRAIL (=rgNDV-NoThaBene-2-TRAIL).

A NDV or rgNDV according to the present invention can be provided in a suitable cell or cell line, for example a HeLa cell line or in cancer cells, or an embryonated egg that is susceptible to a NDV infection.

In these infected cells the virus is grown to sufficient quantities, and preferably under sufficient conditions such that the virus is free from exogenous contamination, and the progeny virus particles are collected by suitable methods well known to those skilled in the art.

In a further aspect the present invention as pertains to a vector comprising any of the nucleic acid sequences as disclosed herein or comprising a nucleic acid sequence encoding a protein as disclosed herein. The protein can for example be a HN protein preferably having an amino acid sequence as shown in SEQ ID No. 32 of the sequence listing, or a F protein preferably having an amino acid sequence as shown in SEQ ID No. 33 or 39 of the sequence listing, or M protein preferably having an amino acid sequence as shown in SEQ ID No. 34 of the sequence listing, or a L protein preferably having an amino acid sequence as shown in SEQ ID No. 35 of the sequence listing. The vector can also encode an NDV according to the present invention.

Moreover, the present invention also pertains to a host cell comprising any of the nucleic acid sequences according to the present invention or a vector according to the present invention.

In a further aspect the present invention is concerned with the medical use of the NDV strains and the recombinant NDV strains (the virus particles) of the present invention. In this regard the invention provides an oncolytic NDV for use in oncological treatment (this term is used herein also replaceable with “for use as a medicament, especially in a method of treatment of cancer”; or the use of said oncolytic NDV in the preparation of a pharmaceutical formulation for use in said method of treatment) in humans (or more generically animals, such as mammalian animals).

In this regard the present invention is first concerned with a NDV or a recombinant NDV according to the present invention for use in medicine. Secondly, the present invention is more particularly concerned with a NDV or a recombinant NDV according to the present invention for use in a method of treating cancer in a subject considered in need thereof.

In particular one or more of the NDV strains and/or the recombinant NDV strains of the present invention are used in a method for treating one or more indications selected from the group consisting of brain tumors, like glioblastoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.

In a preferred embodiment of the present invention the subject to be treated is a mammal, particularly a mammalian animal or a human subject. Still more preferred the one or more oncolytic NDV strains of the present invention may be used for the treatment of adults and/or children, preferably human adults and/or human children.

In one embodiment only one NDV or rgNDV strain or recombinant NDV or recombinant rgNDV strain according to the present invention is used in a method for treating cancer, in particular a cancer selected from the specific cancers as described and/or mentioned in the claims herein. Alternatively in another preferred embodiment of the present invention a combination of at least two of the NDV or rgNDV strains according to the present invention, which are preferably recombinant, is used in a method for treating cancer, in particular a cancer selected from the specific cancers as described and/or mentioned in the claims herein. In particular it is preferred to use a combination of virus particles, wherein each virus particle encodes and expresses another foreign gene, part or variant thereof selected from the group of the foreign genes according to group A or group B. The use of such a combination of foreign genes results in an active combination of proteins beneficially improving the immunological response with the cancer.

Also within the scope of the present invention is the use of one or more NDV or rgNDV strains according to the present invention in combination with one or more other therapies suitable for the treatment of cancer. In a preferred embodiment the one or more NDV or rgNDV strains or a pharmaceutical formulation comprising the same is used in a method for treating cancer, especially a cancer selected from the specific cancers as described and/or mentioned in the claims herein.

The one or more NDV or rgNDV strains and one or more other therapies can be used concurrently or sequentially. In certain embodiments, the one or more NDV or rgNDV and the one or more other therapies are administered in the same (“fixed”) pharmaceutical formulation. In other embodiments, the one or more NDV or rgNDV strains and the one or more other therapies are administered in different formulations. The one or more NDV or rgNDV strains of the present invention and the one or more other therapies can be administered by the same or different routes of administration to the subject considered in need thereof. Another therapy within the meaning of the present invention also comprises an administration of other oncolytical virus strains, for example recombinant NDV or rgNDV strains encoding and expressing foreign genes other than those as disclosed within the present invention or having other mutations in the NDV genome. Particularly preferred NDV strains and rgNDV which may be used in combination are disclosed in the copending European patent application no. 18166400.4 and the copending European patent application no. 19154204.2. For details of these strains reference to these patent applications is made.

Particularly preferred is the combined use of a recombinant NDV or rgNDV strain encoding and expressing as foreign genes a gene encoding an antibody directed to protein PD-1 or an antigen-binding part directed to protein PD-1 (anti-PD-1), preferably a gene encoding Nivolumab, an antigen-binding part of Nivolumab, a variant of Nivolumab or a variant of an antigen-binding part of Nivolumab, a recombinant NDV or rgNDV strain encoding and expressing as foreign genes a gene encoding an antibody directed to the surface protein CTLA-4 or an antigen-binding part directed to protein CTLA-4 (anti-CTLA-4), preferably a gene encoding Ipilimumab, an antigen-binding part of Ipilimumab, a variant of Ipilimumab or a variant of an antigen-binding part of Ipilimumab, and a recombinant NDV or rgNDV strain encoding and expressing as foreign genes a gene encoding an antibody directed to protein PD-L1 or an antigen-binding part directed to protein PD-L1 (anti-PD-L1), preferably a gene encoding Atezolizumab, an antigen-binding part of Atezolizumab, a variant of Atezolizumab or a variant of an antigen-binding part of Atezolizumab. Such a combination results in decreased side effects, as compared to directly administering the respective proteins to a patient in need thereof. For example the combination of the following NDV strains may be used in accordance with the present invention:
NDV-Mut HN(F277L)/M(G165W)-Nivolumab,
NDV-Mut HN(F277L)/M(G165W)-Ipilimumab, and
NDV-Mut HN(F277L)/M(G165W)/F(F117S)/F(F190L)/L(V757I)/L(F1551S)/L(R1700L)-Atezolizumab or
NDV-Mut HN(F277L)/M(G165W)/F(F117S)/F(F190L)/L(V757I)/L(F1551S)/L(R1700L)-Nivolumab,
NDV-Mut HN(F277L)/M(G165W)/F(F117S)/F(F190L)/L(V757I)/L(F1551S)/L(R1700L)-Ipilimumab, and
NDV-Mut HN(F277L)/M(G165W)/F(F117S)/F(F190L)/L(V757I)/L(F1551S)/L(R1700L)-Atezolizumab

In a further aspect the present invention also provides a pharmaceutical formulation comprising a NDV strain, preferably an rgNDV, as described herein, without or preferably with at least one pharmaceutically acceptable carrier material.

For the indications mentioned herein, the appropriate dosage will, of course, vary depending upon, for example, the particular molecule of the invention to be employed, the mode of administration and the nature and severity of the condition being treated. In general, the dosage, in one embodiment of the invention, can preferably be in the range of 10⁷ to 10⁹ pfu per dose.

The NDV strains according to the present invention provide on the one hand an improved replication capacity of the NDV particles in cancer cells and a good or even an improved safety profile, preferably in humans, which is associated with increased cancer cell lysis and increased anti-cancer activity, because the recombinant and mutated NDV strains of the present invention are still selective for cancer cells. The low to sero activity towards normal cells is associated with an increased therapeutic window or safety margin, as is commonly determined for cancer therapeutics. Beside the virological activity the recombinant mutated NDV strains of the present invention are in addition beneficial for the treatment of cancer from an immunological point of view. This is because the foreign gene carried by NDV strains of the present invention beneficially influences the ability of the immune system of the subject to be treated to attack and destroy cancer cells. Advantageously the foreign gene is expressed inside the tumor to be treated, that is directly in the side of need.

The pharmaceutical formulations or pharmaceutical compositions of the present invention may be manufactured in any conventional manner, which are known to those skilled in the art. For example a pharmaceutical composition according to the present invention comprising a recombinant virus particle as described within this description can be provided in a dried, preferably in a lyophilized form and can be complemented with a suitable solvent, e.g. an aqueous carrier for injection at the time when used for administration. The dried form is very stable. Thus, in one embodiment the pharmaceutical composition is present as a dried form, still more preferred in a lyophilized form, and is reconstituted before administration by adding a suitable solvent. That is the present invention also relates to the above described dried form of the pharmaceutical composition for use as a medicament, preferably for use in a method of treating cancer. The process parameters for drying, preferably lyophilisation must be chosen such that the dried/lyophilized product contains live, replication competent viral particles, which can be grown to standardized titers, when needed. A person skilled in the art knows how to set these parameters.

For administration of a dried form, the dried form is directly dissolved in a suitable solvent or pharmaceutically acceptable carrier for injection and/or stabilization of the NDV or rg NDV and/or for improving its delivery to cancer cells. The solvent or carrier is preferably an aqueous carrier, for example sterile water for injection or sterile buffered physiological saline or another, preferably isotonic buffer system having a pH in the range of 7.2 to 7.6. The reconstitution can be performed ideally at or close to the intended time of administration in order to avoid any contaminations with microbes, etc. The skilled person is familiar with the handling of pharmaceutical compositions for reconstitution and reconstituted solutions.

In another embodiment, the present invention is concerned with pharmaceutical formulations comprising cancer cells infected with a transgene-expressing NDV or transgene-expressing rgNDV strain as described herein, and a pharmaceutically acceptable carrier. In specific embodiments, the cancer cells have been treated with gamma radiation prior to incorporation into the pharmaceutical formulation. In specific embodiments, the cancer cells have been treated with gamma radiation before infection with the NDV strain (e.g., rgNDV). In other specific embodiments, the cancer cells have been treated with gamma radiation after infection with the NDV (e.g., rgNDV). In another embodiment, presented herein are pharmaceutical formulations comprising a protein concentrate from lysed NDV-infected cancer cells (e.g., rgNDV infected cancer cells), and a pharmaceutically acceptable carrier.

The pharmaceutical formulation of the present invention can be prepared by a method comprising or consisting of the steps of:
propagating an NDV, preferably an rgNDV, according to the present invention in at least one cell or embryonated egg that is susceptible to a NDV infection;
collecting the progeny virus particles, wherein the virus is grown to sufficient quantities and under sufficient conditions that the virus is free from exogenous contamination, such that the progeny virus is suitable for formulation into a pharmaceutical formulation; and
preferably manufacturing a pharmaceutical formulation comprising said progeny virus particles in the absence of or after addition of at least one pharmaceutically acceptable carrier material.

Thus in a further aspect the present invention also provides a method for producing the above mentioned pharmaceutical formulation. It is also within the scope of the present invention that for manufacturing the said pharmaceutical formulation a nucleic acid according to the present invention is used, from which virus particles can be rescued which are then propagated in at least one cell or embryonated egg that is susceptible to a NDV infection.

The step of collecting progeny virus particles may in one embodiment also comprise a step of processing the collected virus-containing material to enrich virus particles and/or to eliminate host cell DNA.

In a further aspect the present invention also provides a cell or cell line or an embryonated egg comprising a NDV, preferably a rgNDV, according to the present invention.

In still a further aspect the present invention is also concerned with a method for treating cancer, especially a cancer selected from the specific cancers as described and/or mentioned in the claims herein, in a subject considered in need thereof. The method for treating cancer utilizes a NDV, a transgene-expressing NDV or transgene-expressing rgNDV described herein or any combination thereof or a pharmaceutical formulation comprising such a NDV or rgNDV or any combination thereof, especially in a therapeutically effective amount. Within the meaning of the present patent application a therapeutically effective amount also includes an effective amount for preventing a cancer, in particular a cancer selected from the specific cancers as described and/or mentioned in the claims herein.

The method of treatment comprises or consists of administering to a subject in need thereof a NDV or an rgNDV according to the present invention or any combination thereof in a sufficient amount for infecting and destroying some or all of the cancer cells. In a specific embodiment the method for treating cancer comprises or consists of infecting a cancer cell in a subject with a NDV or rgNDV of the present invention or any combination thereof or with a pharmaceutical composition of the present invention. In another embodiment the one or more NDV or rgNDV or a pharmaceutical composition comprising the same is administered to a subject in need thereof by intravenous, intra-arterial, intratumoral, intramuscular, intradermal, subcutaneous, or any other medically relevant route of administration. According to another embodiment of the present invention the NDV or rgNDV according to the present invention or any combination thereof is administering to a subject in need thereof by administering cancer cells infected with a NDV or rgNDV according to the present invention, especially in a therapeutically effective amount, or pharmaceutical formulation thereof by intravenous, intra-arterial, intratumoral, intramuscular, intradermal, subcutaneous, or any other medically relevant route of administration. In specific embodiments, the cancer cells have been treated with gamma radiation prior to administration to the subject or incorporation into the pharmaceutical formulation. In still another embodiment, a method for treating cancer comprises or consists of administering to a subject in need thereof protein concentrates or plasma membrane fragments from cancer cells infected with a NDV or rgNDV or a pharmaceutical formulation according to the present invention.

In a particular preferred embodiment the method for treating a cancer, in particular a cancer selected from the specific cancers as described and/or mentioned in the claims herein, comprises administering a mixture of at least two recombinant and mutated NDV or rgNDV strains according to the present invention. In particular it is preferred to use a combination of virus particles, wherein each virus particle encodes and expresses another foreign gene, preferably a foreign gene according to group A. The administration of such a combination of foreign genes results in an active combination of proteins beneficially improving the immunological response with the cancer. The administration of this mixture of NDVs or rgNDVs according to the present invention can be achieved as specified supra.

Also within the scope of the present invention is a method of treating cancer, especially a cancer selected from the specific cancers as described and/or mentioned in the claims herein, which utilizes a NDV or rgNDV according to the present invention in combination with one or more other therapies. In a preferred embodiment the NDV or rgNDV or a pharmaceutical formulation comprising the same is administered to a subject in need thereof in a therapeutically effective amount. The NDV or rgNDV and one or more other therapies can be administered concurrently or sequentially to the subject (meaning they are jointly therapeutically active). In certain embodiments, the NDV or rgNDV and one or more other therapies are administered in the same (“fixed”) pharmaceutical formulation. In other embodiments, the NDV or rgNDV and one or more other therapies are administered in different formulations. The NDV or rgNDV of the present invention and one or more other therapies can be administered by the same or different routes of administration to the subject considered in need thereof. Another therapy within the meaning of the present invention also comprises an administration of other oncolytical virus strains, for example recombinant NDV or rgNDV strains encoding and expressing foreign genes other than those as disclosed within the present invention or having other mutations in the NDV genome. Particularly preferred NDV strains and rgNDV which may be used in combination are disclosed in the copending

European patent application no. 18166400.4 and the copending European patent application no. 19154204.2. For details of these strains reference to these patent applications is made. Particularly preferred is the combined use of recombinant NDV or rgNDV strains encoding and expressing as foreign genes anti-PD-1, preferably Nivolumab, anti-CTLA-4, preferably Ipilimumab, anti-PD-L1, preferably

Atezolizumab, or parts or variants thereof. For example the following combinations are in accordance with the present invention:
NDV-Mut HN(F277L)/M(G165W)-Nivolumab,
NDV-Mut HN(F277L)/M(G165W)-Ipilimumab, and
NDV-Mut HN(F277L)/M(G165W)/F(F117S)/F(F190L)/L(V757I)/L(F1551S)/L(R1700L)-Atezolizumab or
NDV-Mut HN(F277L)/M(G165W)/F(F117S)/F(F190L)/L(V757I)/L(F1551S)/L(R1700L)-Nivolumab,
NDV-Mut HN(F277L)/M(G165W)/F(F117S)/F(F190L)/L(V757I)/L(F1551S)/L(R1700L)-Ipilimumab, and
NDV-Mut HN(F277L)/M(G165W)/F(F117S)/F(F190L)/L(V757I)/L(F1551S)/L(R1700L)-Atezolizumab

Another therapy may also comprise or may be radiotherapy for cancer.

### FIGURES

FIG. 1 is a schematic presentation of the NDV reverse genetics system. The upper part shows the composition of the full-length cDNA plasmid which contains the full-length NDV cDNA (encoding the nucleocapsid protein (NP), the phosphoprotein (P), the matrix protein (M), the fusion protein (F), the hemagglutinin-neuraminidase (HN), and the RNA-dependent RNA polymerase (L)) cloned behind the bacteriophage T7 RNA Polymerase promoter (T7P; yellow triangle) and followed by a ribozyme sequence (Rz) and T7 transcription termination signal (T7T).

A suitable host cell (shaded round-cornered box) is infected with a recombinant Fowlpox virus that expresses T7 DNA-dependent RNA polymerase (Fowlpox-T7) and subsequently co-transfected with the full-length cDNA plasmid and three helper plasmids containing the genes encoding the NDV NP, P and L proteins, respectively. Transcription of the full-length cDNA results in the generation of the NDV antigenome RNA which is encapsidated by NP protein then transcribed and replicated by the RNA-leading to the generation of infectious NDV.

FIG. 2 is a schematic presentation of the genome of the rgNDV-mutants according to the present invention. The figure shows the position of the respective foreign gene which is inserted as extra transcription unit into the rgNDV-mutant genome between the P and M genes.

FIG. 3 shows growth kinetics in HeLa cells. HeLa cells were infected at a multiplicity of infection (MOI) of 0.01 with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV-Mut HN(F277L)/M(G165W); rgMTH68: NDV strain MTH-68/H derived by reverse genetics from cloned full-length cDNA; rgMutHu: NDV strain NDV-Mut HN(F277L)/M(G165W) derived by reverse genetics from cloned full-length cDNA; rgMutHu(HNL277F): rgMutHu in which the amino acid mutation at position 277 in the HN gene was converted from L back to F; rgMutHu(MW165G): rgMutHu in which the amino acid mutation at position 165 in the M gene was converted from W back to G.

FIG. 4 shows the expression of CD80 in a recombinant Nothabene-2-strain. FIG. 4a shows the recombinant strain, FIG. 4b is the negative control. For detection an anti-CD80 antibody was used.

FIG. 5 shows syncytium formation due to the F(L289A) mutation. FIG. 5a is rgNothabene-2-F2L3+F(L289A) (i.e. F3L3) according to the present invention, FIG. 5b is rgNothabene-2-F2L3.

### EXAMPLES

### Example 1. Nucleotide Sequence Analysis of Mutant NDV-Mut HN(F277L)/M(G165W)

We identified a spontaneous mutant of an oncolytic NDV strain MTH-68/H (Csatary et al., 1999, Anticancer Res. 19:635-638; further called MTH68). The replication capacity of the mutant strain (designated NDV-Mut HN(F277L)/M(G165W) in a variety of human neoplastic cell lines, as well as autologous primary tumors, is greatly enhanced as compared to the original MTH-68/H strain (also referred to as MTH68 strain). We analyzed its nucleotide sequence and found that, compared to MTH68, NDV-HN(F277L)/M(G165W) has two nucleotide mutations, one leading to an amino acid substitution in the M protein (G165W) and the other in the HN protein (F277L).

### Example 2. A Reverse Genetics System that Allows Genetic Modification of NDV-Strains

2.1 Reverse Genetics

In order to be able to genetically modify the genome of an RNA virus such as NDV, a manipulatable genetic system must be developed that uses a copy of the full viral RNA (vRNA) genome in the form of DNA. This full-length cDNA is amenable to genetic modification by using recombinant DNA techniques. The authentic or modified cDNA can be converted back into vRNA in cells, which in the presence of the viral replication proteins results in the production of a new modified infectious virus. Such ‘reverse genetics systems’ have been developed in the last few decades for different classes of RNA viruses. This system enables the rapid and facile introduction of mutations and deletions and the insertion of a transgene transcriptional unit, thereby enabling the changing of the biological properties of the virus.

Reverse genetics systems for several NDV strains, including lentogenic as well as velogenic strains, were developed by the Central Veterinary Institute (CVI), part of Wageningen University and Research, currently Wageningen Bioveterinary Research (WBVR) under the supervision of Dr. Ben Peeters (Peeters et al., 1999, J. Virol. 73:5001-9; de Leeuw et al., 2005, J. Gen. Virol. 86:1759-69; Dortmans et al., 2009, J. Gen. Virol. 90:2746-50). In order to generate a reverse genetics system for providing the NDV nucleic acids and strains according to the present invention, a similar approach was used. Details of the procedure can be found in the above cited papers and in the paragraphs below. Briefly, the system consists of 4 components, i.e., a transcription plasmid containing the full-length (either authentic or genetically modified) cDNA of the virus, which is used to generate the vRNA, and 3 expression plasmids (‘helper plasmids’) containing the NP, P and L genes of NDV respectively, which are used to generate the vRNA-replication complex (consisting of NP, P and L proteins). Transcription of the cDNA (i.e. conversion of the cDNA into vRNA) and expression of the NP, P and L genes by the helper plasmids is driven by a T7 promoter. The corresponding T7 DNA-dependent RNA polymerase (T7-RNAPol) is provided by a helper-virus (Fowlpox-T7).

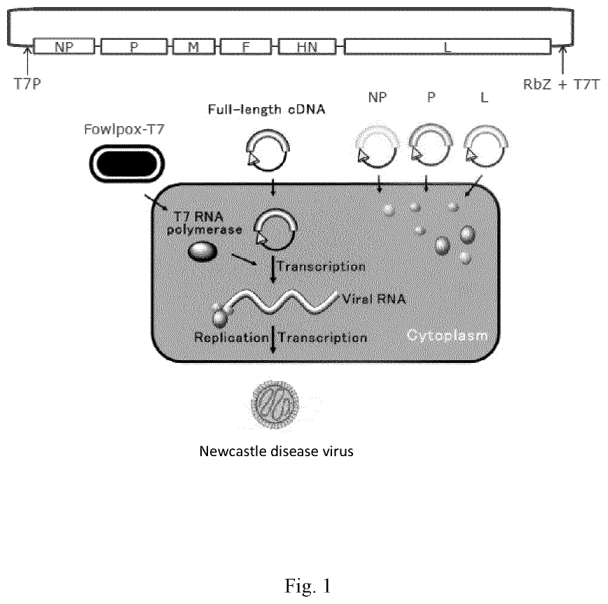

In order to rescue virus, the 4 plasmids are co-transfected into Fowlpox-T7 infected cells (FIG. 1). Three to five days after transfection, the supernatant is inoculated into specific-pathogen-free embryonated chicken eggs (ECE) and incubated for 3 days. Infectious virus that is produced by transfected cells will replicate in the ECE and progeny virus can be harvested from the allantois fluid.

In order to develop a reverse genetics system for NDV the following steps were followed:
Generation of sub-genomic NDV and foreign gene cDNA's by RT-PCR
Assembly of full-length cDNA in a transcription vector
Cloning of each of the NP, P and L genes into an expression vector
Verify nucleotide sequence of full-length cDNA and helper-plasmids
Repair nucleotide differences resulting from the cloning procedure, if necessary
Rescue of infectious virus from cDNA using co-transfection (FIG. 1)

2.2 Construction of Full-Length NDV-Mut HN(F277L)/M(G165W) cDNA, NDV-Mut HN(F277L)/M(G165W)/F(F117S)/F(F190L)/L(V757I)/L(F1551S)/L(R1700L) and Helper Plasmids

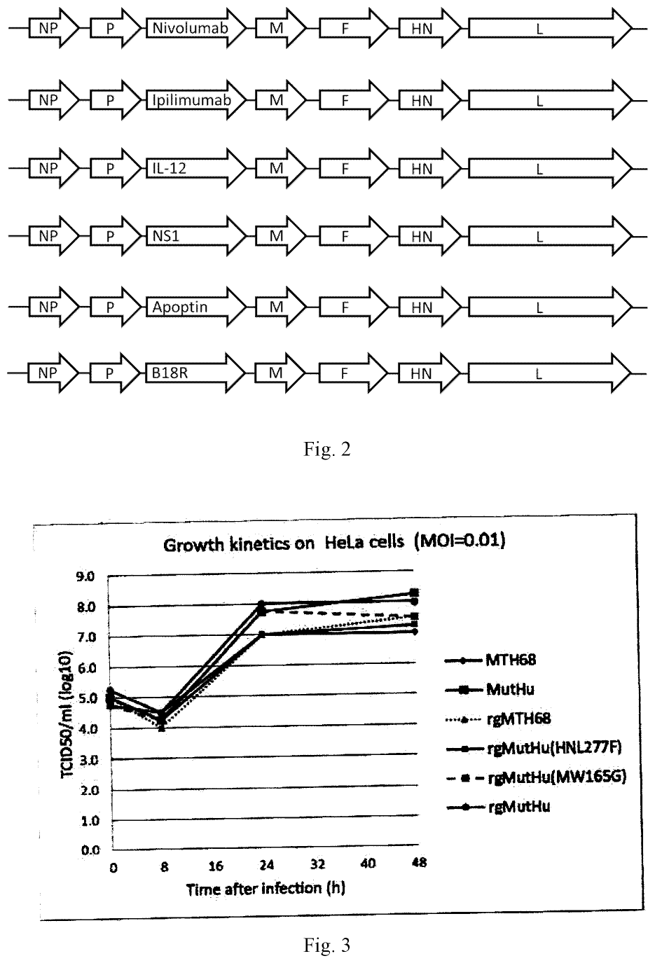

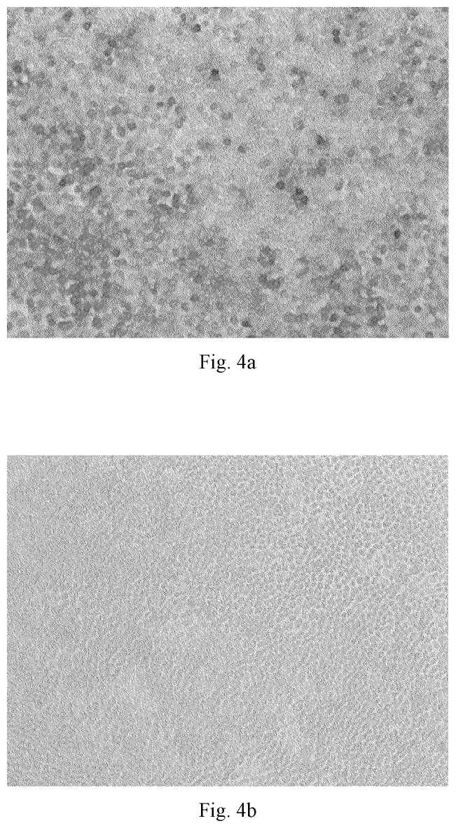

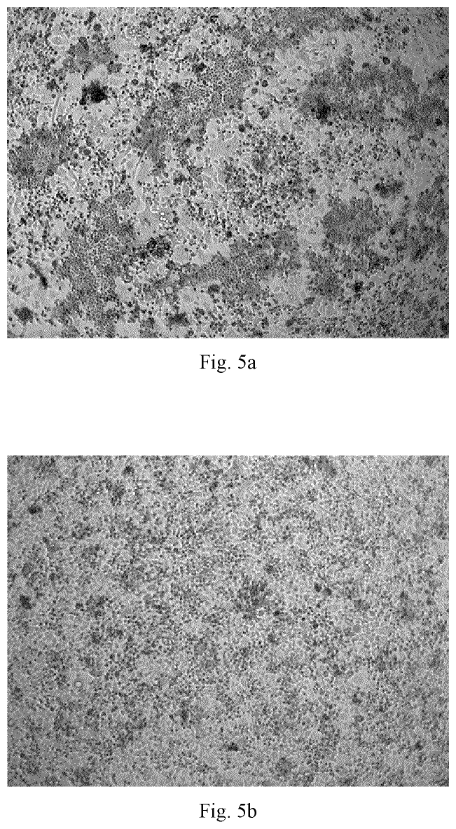

NDV-Mut HN(F277L)/M(G165W) and NDV-Mut HN(F277L)/M(G165W)/F(F117S)/F(F190L)/L(V757I)/L(F1551S)/L(R1700L) (passage 28 HeLa cells) were used for the isolation of vRNA using standard procedures. The vRNA was used to generate first-strand cDNA by means of Reverse Transcriptase followed by PCR to generate 4 sub-genomic cDNA fragments (designated C1, C2, C3 and C8). The full-length cDNA of NDV-MutHu was assembled from these fragments and cloned in the transcription vector pOLTV5 (Peeters et al., 1999, J. Virol. 73:5001-5009) by a combination of In-Fusion® cloning and classical cloning using restriction enzymes. An overview of the procedure is shown in FIG. 2, and further details can be found in Appendix 1 of this reference. The NP, P and L-genes of NDV-MutHu were obtained by RT-PCR (Appendix 1) and cloned in the expression plasmid pCVI which was derived by deletion of a ClaI restriction fragment from pCI-neo (Promega).

2.3 Nucleotide Sequence Analysis

Nucleotide sequence analysis was used to verify that the sequence of pFL-NDV Mut HN(F277L)/M(G165W) and pFL-NDV Mut HN(F277L)/M(G165W)/F(F117S)/F(F190L)/L(V757I)/L(F1551S)/L(R1700L) were correct. A few nucleotides which differed from the Reference sequence were repaired. Silent mutations (i.e., not leading to an amino acid change) may be left unchanged.

2.4 Rescue of Infectious Virus from pFL-NDV Mut HN(F277L)/M(G165W)

In order to generate infectious virus, we used the co-transfection system described above (and illustrated in FIG. 1) to transfect QM5 cells (derived from Quail) using plasmid pFL-NDV_Mut HN(F277L)/M(G165W) and the helper plasmids pCVI-NP^{Mut HN(F277L)/M(G165W)}, pCVI-P^{Mut HN(F277L)/M(G165W)} and pCVI-L^{Mut HN(F277L)/M(G165W)}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W). NDV-Mut HN(F277L)/M(G165W) and rgMut HN(F277L)/M(G165W) have similar growth kinetics in HeLa cells.

2.5 Rescue of Infectious Virus from pFL-NDV Mut HN(F277L)/M(G165W)/F(F117S)/F(F190L)/L(V757I)/L(F1551S)/L(R1700L)

In order to generate infectious virus, we used the co-transfection system described above (and illustrated in FIG. 1) to transfect QM5 cells (derived from Quail) using plasmid pFL-NDV Mut HN(F277L)/M(G165W)/F(F117S)/F(F190L)/L(V757I)/L(F1551S)/L(R1700L) and the helper plasmids pCVI-NP^{Mut HN(F277L)/M(G165W)/F(F117S)/F(F190L)/L(V757I)/L(F1551S)/L(R1700L)}, pCVI-P^{Mut HN(F277L)/M(G165W)/F(F117S)/F(F190L)/L(V757I)/L(F1551S)/L(R1700L)} and pCVI-L^{Mut HN(F277L)/M(G165W)/F(F117S)/F(F190L)/L(V757I)/L(F1551S)/L(R1700L)}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W)/F(F117S)/F(F190L)/L(V757I)/L(F1551S)/L(R1700L).

### Example 3. Identify Whether One or Both of the Amino Acid Substitutions in Mut HN(F277L)/M(G165W) are Responsible for the Difference in Growth Kinetics Between Mut HN(F277L)/M(G165W) and the Parent Strain MTH68

3.1 Growth Kinetics in HeLa Cells

The rescued rg-viruses (Table 1) as well as the original Mut HN(F277L)/M(G165W) and MTH68 viruses were used to determine their growth-kinetics in HeLa cells. Briefly, 4×10⁶ HeLa cells were seeded in 25 cm² cell culture flasks and grown overnight. The cells were infected using a MOI of 0.01 (i.e., 1 infectious virus particle per 100 cells), and at 8, 24 and 48 hours after infection the virus titer in the supernatant was determined by end-point titration on QM5 cells.

The data (FIG. 3) indicate that strains Mut HN(F277L)/M(G165W) and rgMut HN(F277L)/M(G165W) yield at least 10-fold higher virus titers than MTH68. Furthermore, the data indicate that the mutation at amino acid position 277 in the HN gene is responsible for this effect. The M mutation does not seem to have an effect. This can be best seen when looking at the virus titers 24 h after infection, or even better when comparing the increase in virus titer between 8h and 24h (the exponential growth phase). The virus titer shows an increase of 3.5 (log 10) for Mut HN(F277L)/M(G165W), rgMut HN(F277L)/M(G165W) and rgMut HN(F277L)/M(W165G), whereas this is 2.5 for MTH68, 2.7 for rgMut HN(L277F) and 3.0 for rgMTH68 (Table 3).

rgMut HN(F277L)/M(W165G) is a strain in which the mutation in the M gene has been restored in accordance with the NDV MTH-68/H.

TABLE 1
Virus titers (log10 TCID50/ml)

Time after infection (h)

| Virus | 0 h | 8 h | 24 h | 48 h |
|---|---|---|---|---|
| MTH68 | 4.8 | 4.5 | 7.0 | 7.0 |
| Mut HN(F277L)/M(G165W) | 5.0 | 4.3 | 7.8 | 8.3 |
| rgMTH68 | 5.0 | 4.0 | 7.0 | 7.5 |
| rgMut HN(L277F) | 5.0 | 4.3 | 7.0 | 7.3 |
| rgMut M(W165G) | 4.8 | 4.3 | 7.8 | 7.5 |
| rgMutHu | 5.3 | 4.5 | 8.0 | 8.0 |

### Example 4. Generation of Recombinant NDV-Strains with Enhanced Oncolytic and Immune Stimulating Properties Due to the Expression of Different Therapeutic Proteins

The recombinant NDV-strains expressing one of the foreign genes of the present invention can be generated by means of the previously established reverse genetics system described above. The respective gene is to be inserted into the full-length cDNA of e.g. NDV-Mut HN(F277L)/M(G165W)/F(F117S)/F(F190L)/L(V757I)/L(F1551S)/L(R1700L) between the P and the M genes. To this end the open reading frames of the foreign genes may be fused via a 2A sequence. The genes were provided with the necessary NDV gene-start and gene-end sequences in order to allow transcription by the vRNA polymerase. FIG. 2 shows the final constellation of the recombinant viruses.

Infectious virus can be rescued, and virus stocks can be prepared by two passages in HeLa cells. Expression of the respective foreign gene can be determined and quantified by using a total human IgG ELISA (Invitrogen).

### REFERENCES

Blach-Olszewska et al., (1977) Why HeLa cells do not produce interferon? Arch Immunol Ther Exp (Warsz) 25:683-91.
Chng et al., (2015) Cleavage efficient 2A peptides for high level monoclonal antibody expression in CHO cells, mAbs, 7:2, 403-412; http://dx.doi.org/10.1080/19420862.2015.1008351.
Enoch et al., (1986) Activation of the Human beta-Interferon Gene Requires an Interferon-Inducible Factor, Mol. Cell. Biol. 6:801-10.
Haryadi et al., (2015) Optimization of Heavy Chain and Light Chain Signal Peptides for High Level Expression of Therapeutic Antibodies in CHO Cells. PLoS ONE 10(2): e0116878. doi:10.1371/journal.pone.0116878.
Schirrmacher, (2015) Oncolytic Newcastle disease virus as a prospective anti-cancer therapy. A biologic agent with potential to break therapy resistance. Expert Opin. Biol. Ther. 15:17 57-71
Zamarin et al., (2014) Localized oncolytic virotherapy overcomes systemic tumor resistance to immune checkpoint blockade immunotherapy. Sci. Transl. Med. 6(226).
Zamarin & Palese, (2017) Oncolytic Newcastle Disease Virus for cancer therapy: old challenges and new directions. Future Microbiol. 7: 347-67.

APPENDIX 1
primers used for the generation of cDNA
fragments and helper-plasmids
cDNA fragments

| | | | Sequence |
|---|---|---|---|
| Fragment | Size | Primer | (5′-3′) |
| C1 | 3.6 kb | Noss-09 | ACGACTCACT |
| | | (SEQ ID | ATAGGaccaa |
| | | No. 40) | acagagaatc |
| | | | cgtgag |
| | | Noss-121 | CCGGGAAGAT |
| | | (SEQ ID | CCAGGgcact |
| | | No. 41) | cttcttgcat |
| | | | gttac |
| C2 | 3.7 kb | Noss-122 | GGGCCTGCCT |
| | | (SEQ ID | CACTAtggtg |
| | | No. 42) | gtaacatgca |
| | | | agaag |
| | | Noss-123 | TGCATGTTAC |
| | | (SEQ ID | CACCAatgtg |
| | | No. 43) | tcattgtatc |
| | | | gcttg |
| C3 | 5.7 kb | Noss-125 | CAAGAAGGGA |
| | | (SEQ ID | GATACgtaat |
| | | No. 44) | atacaagcga |
| | | | tacaatg |
| | | Noss-126 | TCGCTTGTAT |
| | | (SEQ ID | ATTACttgtt |
| | | No. 45) | gtagcaaaga |
| | | | gcacc |
| C8 | 2.0 kb | Noss-133 | GGCCTGGATC |
| | | (SEQ ID | TTCCCattat |
| | | No. 46) | gctgtctgta |
| | | | tacggtgc |
| | | Noss-10 | ATGCCATGCC |
| | | (SEQ ID | GACCCaccaa |
| | | No. 47) | acaaagactt |
| | | | ggtgaatg |
| iPCR | 2.5 kb | Noss-17 | CCTATAGTGA |
| pOLTV5 | | (SEQ ID | GTCGTATTAA |
| (StuI) | | No. 48) | TTTC |
| | | Noss-128 | CCTGGATCTT |
| | | (SEQ ID | CCCGGGTCGG |
| | | No. 49) | |
| iPCR | 2.5 kb | Noss-137 | GGGTCGGCAT |
| pOLTV5 | | (SEQ ID | GGCATCTCCA |
| (Smal) | | No. 50) | CC |
| | | Noss-138 | GGGAAGATCC |
| | | (SEQ ID | AGGCCTATAG |
| | | No. 51) | TG |

| Helper-plasmids (generated by | | |
|---|---|---|
| In-Fusion® cloning in pCVI) | | |
| Gene | primer | sequence |
| NP | Noss-22 | CTCTAGAGTC |
| | (SEQ ID | GACCCttctg |
| | No. 52) | ccaacatgtc |
| | | ttccg |
| | Koss-23 | GGGAAGCGGC |
| | (SEQ ID | CGCCCgtcgg |
| | No. 53) | tcagtatccc |
| | | cagtc |
| | Noss-24 | CTCTAGAGTC |
| | (SEQ ID | GACCCcagag |
| | No. 54) | tgaagatggc |
| | | caccttc |
| P | Noss- | GGGAAGCGGC |
| | 25n | CGCCCgtagt |
| | (SEQ ID | agtgatcagc |
| | No. 55) | cattc |
| L | Noss-26 | CTCTAGAGTC |
| | (SEQ ID | GACCCgggta |
| | No. 56) | ggacatggcg |
| | | ggctc |
| | Noss-27 | GGGAAGCGGC |
| | (SEQ ID | CGCCCtgcct |
| | No. 57) | ttaagagtca |
| | | cagttac |

What is claimed is:

1. A Newcastle Disease Virus (NDV), derived from NDV strain MTH-68/H according to SEQ ID No. 1, comprising a viral genome comprising a nucleic acid comprising
a nucleic acid sequence encoding a hemagglutinin-neuramidase protein (HN protein) in which phenylalanine (F) at position 277 is substituted to leucine (L), and
a nucleic acid sequence encoding a fusion protein (F protein) in which phenylalanine (F) is substituted to serine (S) at position 117 of the F protein, phenylalanine (F) is substituted to leucine (L) at position 190 of the F protein, and leucine (L) is substituted to alanine (A) at position 289 of the F protein.

2. The NDV according to claim 1, wherein the viral genome further comprises a nucleic acid comprising a nucleic acid sequence encoding a matrix protein (M protein) in which glycine (G) is substituted to tryptophane (W) at position 165 of the M protein.

3. The NDV according to claim 1, wherein the viral genome further comprises a nucleic acid comprising a nucleic acid sequence encoding a large polymerase protein (L protein) in which
valine (V) is substituted to isoleucine (I) at position 757 of the L protein, and/or
phenylalanine (F) is substituted to serine (S) at position 1551 of the L protein, and/or
arginine (R) is substituted to leucine (L) at position 1700 of the L protein.

4. The NDV according to claim 3, wherein in the large polymerase protein (L protein) encoded by the nucleic acid sequence
tyrosine (Y) is substituted to histidine (H) at position 1717 of the L protein, and/or
glutamic acid (E) is substituted to lysine (K) at position 1910 of the L protein.

5. The NDV according to claim 1, wherein the nucleic acid is at least 70% identical to a nucleic acid sequence according to SEQ ID No. 1 to SEQ ID No. 3 of the sequence listing, wherein the sequence identity is determined after best alignment of the sequence of interest with the respective reference sequence.

6. The NDV according to claim 1, wherein
the HN protein comprises or consists of the amino acid sequence according to SEQ ID No. 32 of the sequence listing, and/or
wherein the F protein comprises or consists of the amino acid sequence according to SEQ ID No. 33 of the sequence listing, and/or
wherein the nucleic acid comprises a nucleic acid sequence encoding a membrane (M) protein which M protein comprises or consists of the amino acid sequence according to SEQ ID No. 34 of the sequence listing, and/or
wherein the nucleic acid comprises a nucleic acid sequence encoding a large polymerase protein which L protein-comprises or consists of the amino acid sequence according to SEQ ID No. 35 of the sequence listing.

7. The NDV according to claim 1, wherein the NDV is a recombinant virus comprising a nucleic acid sequence encoding at least one foreign gene, the at least one foreign gene being selected from the group consisting of:
a gene encoding Atezolizumab, a variant of Atezolizumab or a variant of an antigen-binding part of Atezolizumab,
a gene encoding Bevacizumab, an antigen-binding part of Bevacizumab, a variant of Bevacizumab or a variant of an antigen-binding part of Bevacizumab,
a gene encoding Lirilumab, an antigen-binding part of Lirilumab, a variant of Lirilumab or a variant of an antigen-binding part of Lirilumab,
a gene encoding Relatlimab, an antigen-binding part of Relatlimab, a variant of Relatlimab or a variant of an antigen-binding part of Relatlimab,
a gene encoding a gene encoding Monalizumab, an antigen-binding part of Monalizumab, a variant of Monalizumab or a variant of an antigen-binding part of Monalizumab,
a gene encoding TRX518, an antigen-binding part of TRX518, a variant of TRX518 or a variant of an antigen-binding part of TRX518,
a gene encoding BMS 986178, an antigen-binding part of BMS 986178, a variant of BMS 986178 or a variant of an antigen-binding part of BMS 986178,
a gene encoding the protein CD40 (cluster of differentiation 40), a part of CD40, a variant of CD40 or a variant of a part of CD40,
a gene encoding the protein CD80, a part of CD80, a variant of CD80 or a variant of a part of CD80,
a gene encoding non-secreting human interleukin 12 t(ns)hIL-12), a part of (ns)hIL-12, a variant of (ns)hIL-12 or a variant of a part of (ns)hIL-12,
a gene encoding a green fluorescent protein or a part of a green fluorescent protein,
a gene encoding Nivolumab, an antigen-binding part of Nivolumab, a variant of Nivolumab or a variant of an antigen-binding part of Nivolumab,
a gene encoding Ipilimumab, an antigen-binding part of Ipilimumab, a variant of Ipilimumab or a variant of an antigen-binding part of Ipilimumab,
a gene encoding interleukin-12 (IL-12), a part of interleukin-12, a variant of interleukin-12 or a variant of a part of interleukin-12,
a gene encoding the non-structural protein NS1 of influenza A virus, a part of the non-structural protein NS1 of influenza A virus, a variant of the non-structural protein NS1 of influenza A virus or a variant of a part of the non-structural protein NS1 of influenza A virus,
a gene encoding Apoptin, a part of Apoptin, a variant of Apoptin or a variant of a part of Apoptin,
a gene encoding the viral protein B18R from vaccinia virus, a part of the viral protein B18R from vaccinia virus, a variant of the viral protein B18R from vaccinia virus or a variant of a part of the viral protein B18R from vaccinia virus,
a gene encoding Theralizumab, an antigen-binding part of Theralizumab, a variant of Theralizumab or a variant of an antigen-binding part of Theralizumab,
a gene encoding an antibody, directed to CD40 or an antigen-binding part directed to CD40 (anti-CD40),
a gene encoding an antibody, directed to CD80 or an antigen-binding part directed to CD80 (anti-CD80),
a gene encoding an antibody directed to CA 15-3 or an antigen-binding part directed to CA 15-3 (anti-CA 15-3), according to SEQ. ID. No. 22 of the sequence listing,
a gene encoding an antibody directed to CA 19-9 or an antigen-binding part directed to CA 19-9 (anti-CA 19-9), according to SEQ. ID. No. 23,
a gene encoding Sofituzumab, an antigen-binding part of Sofituzumab, a variant of Sofituzumab or a variant of an antigen-binding part of Sofituzumab,
a gene encoding Cetuximab, an antigen-binding part of Cetuximab, a variant of Cetuximab or a variant of an antigen-binding part of Cetuximab,
a gene encoding Trastuzumab, an antigen-binding part of Trastuzumab, a variant of Trastuzumab or a variant of an antigen-binding part of Trastuzumab,
a gene encoding the antibody BIL=3s, an antigen-binding part of BIL=3 s, a variant of BIL=3 s or a variant of an antigen-binding part of BIL=3s,
a gene encoding the antibody J591, an antigen-binding part of J591, a variant of J591 or a variant of an antigen-binding part of J591,
a gene encoding Ramucirumab, an antigen-binding part of Ramucirumab, a variant of Ramucirumab or a variant of an antigen-binding part of Ramucirumab,
a gene encoding the protein TRAIL, a part of TRAIL, a variant of TRAIL or a variant of a part of TRAIL, and
any combination of these genes, parts or variants.

8. A nucleic acid comprising a nucleic acid sequence encoding a Newcastle Disease Virus (NDV) according to claim 1, the nucleic acid comprising the nucleic acid sequence encoding the hemagglutinin-neuramidase (HN) protein, where the nucleic acid sequence encodes HN^{F277L}, and
the nucleic acid sequence encoding a fusion (F) protein, where the nucleic acid sequence encodes F^{F117S}, F^{F190L}, and F^{L289A}.

9. (canceled)

10. (canceled)

11. (canceled)

12. (canceled)

13. (canceled)

14. A recombinant Newcastle Disease Virus (NDV), derived from NDV strain MTH-68/H according to SEQ ID No. 1, wherein the recombinant NDV comprises a viral genome comprising a nucleic acid comprising a nucleic acid sequence encoding at least one foreign gene, the at least one foreign gene being selected from the group consisting of:
a gene encoding an antibody directed to protein PD-1 or an antigen-binding part directed to protein PD-1 (anti-PD-1),
a gene encoding an antibody directed to the surface protein CTLA-4 or an antigen-binding part directed to protein CTLA-4 (anti-CTLA-4),
a gene encoding a protein which improves the cellular immune response and the ability of T cells to enter tumor cells, or a part thereof which improves the cellular immune response and the ability of T cells to enter tumor cells,
a gene encoding a protein with the ability to modulate the virus replication cycle, or a part thereof with the ability to modulate the virus replication cycle,
a gene encoding a protein with the ability to selectively induce apoptosis in human tumor cells, but not in normal human cells, or a part thereof with the ability to selectively induce apoptosis in human tumor cells, but not in normal human cells,
a gene encoding a protein that reduces or inhibits IFN expression, or a part thereof that reduces or inhibits IFN expression
a gene encoding the protein CD40 (cluster of differentiation 40), a part of CD40, a variant of CD40 or a variant of a part of CD40,
a gene encoding an antibody directed to CD28 or an antigen-binding part directed to CD28 (anti-CD28),
a gene encoding an antibody directed to CD40 or an antigen-binding part directed to CD40 (anti-CD40),
a gene encoding an antibody directed to CD80 or an antigen-binding part directed to CD80 (anti-CD80),
a gene encoding an antibody directed to CA 15-3 or an antigen-binding part directed to CA 15-3 (anti-CA 15-3),
a gene encoding an antibody directed to CA 19-9 or an antigen-binding part directed to CA 19-9 (anti-CA 19-9),
a gene encoding an antibody directed to CA 125 or an antigen-binding part directed to CA 125 (anti-CA 125),
a gene encoding an antibody directed to EGFR or an antigen-binding part directed to EGFR (anti-EGFR),
a gene encoding an antibody directed to HER2 or an antigen-binding part directed to HER2 (anti-HER2),
a gene encoding an antibody directed to nfP2X₇ or an antigen-binding part directed to nfP2X₇ (anti-nfP2X₇),
a gene encoding an antibody directed to PSMA or an antigen-binding part directed to PSMA (anti-PSMA),
a gene encoding an antibody, directed to VEGFR or an antigen-binding part directed to VEGFR (anti-VEGFR),
a gene encoding a protein that induces the process of apoptosis by binding to a death receptor or a part thereof that induces the process of apoptosis by binding to a death receptor,
and any combination of these genes, parts or variants, wherein
the viral genome comprises a nucleic acid sequence encoding a hemagglutinin-neuramidase protein (HN protein) in which phenylalanine (F) is substituted to leucine (L) at position 277, and
a nucleic acid sequence encoding a fusion protein (F protein) in which phenylalanine (F) is substituted to serine (S) at position 117 of the F protein, and phenylalanine (F) is substituted to leucine (L) at position 190 of the F protein.

15. The recombinant NDV according to claim 14, wherein the viral genome comprises a nucleic acid sequence encoding a matrix protein (M protein) in which glycine (G) is substituted to tryptophane (W) at position 165 of the M protein.

16. The recombinant NDV according to claim 14, wherein the viral genome comprises a nucleic acid sequence encoding a large polymerase protein (L protein) in which
valine (V) is substituted to isoleucine (I) at position 757 of the L protein, and/or
phenylalanine (F) is substituted to serine (S) at position 1551 of the L protein, and/or
arginine (R) is substituted to leucine (L) at position 1700 of the L protein.

17. The recombinant NDV according to claim 16, wherein in the large polymerase protein (L protein) encoded by the nucleic acid sequence
tyrosine (Y) is substituted to histidine (H) at position 1717 of the L protein, and/or
glutamic acid (E) is substituted to lysine (K) at position 1910 of the L protein.

18. The recombinant NDV according to claim 14, wherein the nucleic acid is at least 70% identical to a nucleic acid sequence according to SEQ ID No. 1, SEQ ID No. 36, SEQ ID No. 37 or SEQ ID No. 38 of the sequence listing, wherein the sequence identity is determined after best alignment of the sequence of interest with the respective reference sequence.

19. The recombinant NDV according to claim 14, wherein
the HN protein comprises or consists of the amino acid sequence according to SEQ ID No. 32 of the sequence listing, and/or
wherein the F protein comprises or consists of the amino acid sequence according to SEQ ID No. 39 of the sequence listing, and/or
wherein the viral genome comprises a nucleic acid sequence encoding a matrix (M) protein, which M protein comprises or consists of the amino acid sequence according to SEQ ID No. 34 of the sequence listing, and/or
wherein the viral genome comprises a nucleic acid sequence encoding a large polymerase (L) protein, which L protein comprises or consists of the amino acid sequence according to SEQ ID No. 35 of the sequence listing.

20. A nucleic acid encoding the recombinant Newcastle Disease Virus (NDV) according to claim 14, the nucleic acid comprising a transgenic construct, wherein said transgenic construct comprises a nucleic acid sequence encoding at least one protein selected from the group consisting of:
an antibody directed to protein PD-1 or an antigen-binding part directed to protein PD-1 (anti-PD-1),
an antibody directed to the surface protein CTLA-4 or an antigen-binding part directed to protein CTLA-4 (anti-CTLA-4),
a protein which improves the cellular immune response and the ability of T cells to enter tumor cells, or a part thereof which improves the cellular immune response and the ability of T cells to enter tumor cells,
a protein with the ability to modulate the virus replication cycle, or a part thereof with the ability to modulate the virus replication cycle,
a protein with the ability to selectively induce apoptosis in human tumor cells, but not in normal human cells, or a part thereof with the ability to selectively induce apoptosis in human tumor cells, but not in normal human cells,
a protein that reduces or inhibits IFN expression such as an IFN-beta receptor, or a part thereof that reduces or inhibits IFN expression such as an IFN-beta receptor,
a protein CD40 (cluster of differentiation 40), a part of CD40, a variant of CD40 or a variant of a part of CD40,
an antibody, directed to CD28 or an antigen-binding part directed to CD28 (anti-CD28),
an antibody, directed to CD40 or an antigen-binding part directed to CD40 (anti-CD40),
an antibody, directed to CD80 or an antigen-binding part directed to CD80 (anti-CD80),
an antibody directed to CA 15-3 or an antigen-binding part directed to CA 15-3 (anti-CA 15-3),
an antibody directed to CA 19-9 or an antigen-binding part directed to CA 19-9 (anti-CA 19-9),
an antibody directed to CA 125 or an antigen-binding part directed to CA 125 (anti-CA 125),
an antibody, directed to EGFR or an antigen-binding part directed to EGFR (anti-EGFR),
an antibody, directed to HER2 or an antigen-binding part directed to HER2 (anti-HER2),
an antibody directed to nfP2X₇ or an antigen-binding part directed to nfP2X₇ (anti-nfP2X₇),
an antibody directed to PSMA or an antigen-binding part directed to PSMA (anti-PSMA),
an antibody, directed to VEGFR or an antigen-binding part directed to VEGFR (anti-VEGFR),
a protein that induces the process of apoptosis by binding to a death receptor, or a part thereof that induces the process of apoptosis by binding to a death receptor,
any combination of these proteins, parts or variants,
wherein the nucleic acid in addition comprises the nucleic acid sequence encoding the hemagglutinin-neuramidase protein (HN protein) with an amino acid substitution at position 277 to an amino acid with a hydrophobic side chain other than phenylalanine, namely HN^{F277L}, and
the nucleic acid sequence encoding the fusion protein (F protein) with an amino acid substitution at position 117 to an amino acid with a hydroxylated side chain, namely F^{F117S}, and with an amino acid substitution at position 190 to an amino acid with an aliphatic side chain, namely F^{F190L}.

21. The nucleic acid according to claim 20, further comprising a nucleic acid sequence encoding a matrix protein (M protein) with an amino acid substitution at position 165 to an amino acid with an aromatic side chain, namely M^{G165W}.

22. (canceled)

23. (canceled)

24. The nucleic acid according to claim 20, wherein the nucleic acid comprises at least one of the nucleic acids according to SEQ ID No. 1, SEQ ID No. 36, SEQ ID No. 37 or SEQ ID No. 38 of the sequence listing or variants thereof, the variants having a sequence identity of at least 75%, to the respective sequence, wherein the sequence identity is determined after best alignment of the sequence of interest with the respective reference sequence.

25. The NDV according to claim 7, wherein the nucleic acid comprising the nucleic acid sequence encoding the at least one foreign gene comprises or is a nucleic acid sequence according to any one the nucleic acid sequences according to SEQ ID No. 5 to 30 of the sequence listing.

26. (canceled)

27. (canceled)

28. A method of treating cancer in a subject considered in need thereof, the method comprising administering the NDV according to claim 1 to the subject, wherein the cancer is selected from the group consisting of brain tumors, bone tumors, soft tissue tumors, gynecological tumors, gastrointestinal tumors, pancreas tumors, prostate tumors, lung tumors, ear tumors, nose tumors, throat tumors, tongue tumors, and skin tumors.

29-31. (canceled)

32. The recombinant NDV according to claim 14, wherein the at least one foreign gene is selected from the group consisting of:
the gene encoding an antibody directed to protein PD-1 or an antigen-binding part directed to protein PD-1 (anti-PD-1), which gene encodes Nivolumab, an antigen-binding part of Nivolumab, a variant of Nivolumab or a variant of an antigen-binding part of Nivolumab,
the gene encoding an antibody directed to the surface protein CTLA-4 or an antigen-binding part directed to protein CTLA-4 (anti-CTLA-4), which gene encodes Ipilimumab, an antigen-binding part of Ipilimumab, a variant of Ipilimumab or a variant of an antigen-binding part of Ipilimumab,
the gene encoding a protein which improves the cellular immune response and the ability of T cells to enter tumor cells, or a part thereof which improves the cellular immune response and the ability of T cells to enter tumor cells, which gene encodes interleukin-12 (IL-12), a part of interleukin-12, a variant of interleukin-12 or a variant of a part of interleukin-12,
the gene encoding a protein with the ability to modulate the virus replication cycle, or a part thereof with the ability to modulate the virus replication cycle, which gene encodes the non-structural protein NS1 of influenza A virus, a part of the non-structural protein NS1 of influenza A virus, a variant of the non-structural protein NS1 of influenza A virus or a variant of a part of the non-structural protein NS1 of influenza A virus,
the gene encoding a protein with the ability to selectively induce apoptosis in human tumor cells, but not in normal human cells, or a part thereof with the ability to selectively induce apoptosis in human tumor cells, but not in normal human cells, which gene encodes Apoptin, a part of Apoptin, a variant of Apoptin or a variant of a part of Apoptin,
the gene encoding a protein that reduces or inhibits IFN expression, or a part thereof that reduces or inhibits IFN expression, which gene encodes the viral protein B18R from vaccinia virus, a part of the viral protein B18R from vaccinia virus, a variant of the viral protein B18R from vaccinia virus or a variant of a part of the viral protein B18R from vaccinia virus,
the gene encoding the protein CD40 (cluster of differentiation 40), a part of CD40, a variant of CD40 or a variant of a part of CD40,
the gene encoding an antibody directed to CD28 or an antigen-binding part directed to CD28 (anti-CD28), which gene encodes Theralizumab, an antigen-binding part of Theralizumab, a variant of Theralizumab or a variant of an antigen-binding part of Theralizumab,
the gene encoding an antibody directed to CA 15-3 or an antigen-binding part directed to CA 15-3 (anti-CA 15-3), having a sequence which comprises or is the nucleic acid sequence SEQ. ID. No. 22 of the sequence listing,
a gene encoding an antibody directed to CA 19-9 or an antigen-binding part directed to CA 19-9 (anti-CA 19-9), having a sequence which comprises or is the nucleic acid sequence SEQ. ID. No. 23 of the sequence listing,
the gene encoding an antibody directed to CA 125 or an antigen-binding part directed to CA 125 (anti-CA 125), which gene encodes Sofituzumab, an antigen-binding part of Sofituzumab, a variant of Sofituzumab or a variant of an antigen-binding part of Sofituzumab,
the gene encoding an antibody directed to EGFR or an antigen-binding part directed to EGFR (anti-EGFR), which gene encodes Cetuximab, an antigen-binding part of Cetuximab, a variant of Cetuximab or a variant of an antigen-binding part of Cetuximab,
the gene encoding an antibody directed to HER2 or an antigen-binding part directed to HER2 (anti-HER2), which gene encodes Trastuzumab, an antigen-binding part of Trastuzumab, a variant of Trastuzumab or a variant of an antigen-binding part of Trastuzumab,
the gene encoding an antibody directed to nfP2X₇ or an antigen-binding part directed to nfP2X₇ (anti-nfP2X₇), which gene encodes BIL=3s, an antigen-binding part of BIL=3 s, a variant of BIL=3s or a variant of an antigen-binding part of BIL=3s,
the gene encoding an antibody directed to PSMA or an antigen-binding part directed to PSMA (anti-PSMA), which gene encodes J591, an antigen-binding part of J591, a variant of J591 or a variant of an antigen-binding part of J591,
the gene encoding an antibody directed to VEGFR or an antigen-binding part directed to VEGFR (anti-VEGFR), which gene encodes Ramucirumab, an antigen-binding part of Ramucirumab, a variant of Ramucirumab or a variant of an antigen-binding part of Ramucirumab,
the gene encoding a protein that induces the process of apoptosis by binding to a death receptor or a part thereof that induces the process of apoptosis by binding to a death receptor which gene encodes TRAIL, a part of TRAIL, a variant of TRAIL or a variant of a part of TRAIL
and any combination of these genes, parts or variants.


SEQUENCE LISTING (57 sequences)

SEQ ID NO: 1 - DNA, 15186 bp, Newcastle disease virus
  STRAIN MTH-68/H
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatccctgggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggtttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 2 - DNA, 15186 bp, Newcastle Disease Virus
  Mut HN(F277L)/M(G165W)/F(F117S)/F(F190L)/ F(L289A)/L(V757I)/L(F1551S)/L(R1700L)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 3 - DNA, 15198 bp, Newcastle Disease Virus
  Mut HN(F277L)/M(G165W)/F(F117S)/F(F190L)/ F(L289A)/L(V757I)/L(F1551S)/L(R1700L)+AfeI
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcgctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 4 - DNA, 12 bp, Artificial Sequence
  12bp insert containing a AfeI restriction site
agcgctgaccga

SEQ ID NO: 5 - DNA, 2238 bp, Artificial Sequence
  transgenic construct/Atezolizumab
ttaagaaaaaatacgggtagaagcagtttgagagctccgttcgccaccatggagttcggcctgtcctgggtgttcctggtcgccctgttccggggagtgcagtgtgaggtgcagctggtcgagtctggggggggcctggtgcagccaggaggctccctgaggctgtcttgcgcagcaagcggcttcaccttttctgacagctggatccactgggtgagacaggcaccaggcaagggcctggagtgggtggcatggatcagcccttacggcggctccacctactatgccgacagcgtgaagggcaggtttacaatctccgccgatacctctaagaacacagcctatctgcagatgaattccctgcgcgccgaggataccgccgtgtactattgcgcacggagacactggcctggaggatttgattactggggccaggggaccctggtgacagtgagctccgcctctaccaagggaccaagcgtgttcccactggcaccttctagcaagtccacctctggaggaacagccgccctgggctgtctggtgaaggattactttcctgagccagtgaccgtgagctggaactccggcgccctgaccagcggagtgcacacattcccagccgtgctgcagtcctctggcctgtactccctgagctccgtggtgaccgtgccctctagctccctgggcacccagacatatatctgcaacgtgaatcacaagccatccaatacaaaggtggacaagaaggtggagcccaagtcttgtgataagacccacacatgcccaccttgtccagcacctgagctgctgggaggaccaagcgtgttcctgtttccacccaagcctaaggacaccctgatgatcagcaggacccctgaggtgacctgcgtggtggtggacgtgtcccacgaggatccagaggtgaagttcaactggtacgtggatggcgtggaggtgcacaatgccaagaccaagcctcgggaggagcagtacgcctccacctatagagtggtgtctgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatctcgcgaggagatgaccaagaaccaggtgagcctgacatgtctggtgaagggcttttatccaagcgacatcgccgtggagtgggagtccaatggccagcccgagaacaattacaagaccacaccccctgtgctggactctgatggcagcttctttctgtattctaagctgaccgtggataagagcaggtggcagcagggcaacgtgttcagctgctccgtgatgcacgaggccctgcacaatcactacacacagaagtctctgagcctgtccccaggcaagaggaggaagaggggaagcggagagggaagaggctccctgctgacctgtggcgacgtggaggagaaccctggcccaatgaagtatctgctgcccacagcagccgccggcctgctgctgctggcagcacagcctgccatggccgatatccagatgacccagagcccatctagcctgtctgccagcgtgggcgacagggtgaccatcacatgcagggcaagccaggacgtgtctaccgccgtggcctggtaccagcagaagcctggcaaggccccaaagctgctgatctactccgcctctttcctgtattctggagtgccaagccgctttagcggatccggatctggaaccgacttcaccctgacaatctcctctctgcagcccgaggattttgccacatactattgccagcagtacctgtatcaccctgccaccttcggccaggggaccaaggtggagatcaagaggaccgtggcagcaccatccgtgttcatctttccaccctctgacgagcagctgaagtccggcacagcctctgtggtgtgcctgctgaacaatttctaccctagagaggccaaggtgcagtggaaggtggataacgccctgcagtccggcaattctcaggagagcgtgaccgagcaggactccaaggattctacatatagcctgagctccaccctgacactgtccaaggccgactacgagaagcacaaggtgtatgcctgtgaggtcacccatcaggggctgtcctcaccagtcacaaaatccttcaatcggggggaatgctaataa

SEQ ID NO: 6 - DNA, 2250 bp, Artificial Sequence
  transgenic construct/anti-VEGF-A
ttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggctacaccttcaccaactacggcatgaactgggtgaggcaggcccccggcaagggcctggagtgggtgggctggatcaacacctacaccggcgagcccacctacgccgccgacttcaagaggaggttcaccttcagcctggacaccagcaagagcaccgcctacctgcagatgaacagcctgagggccgaggacaccgccgtgtactactgcgccaagtacccccactactacggcagcagccactggtacttcgacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagcgccagccaggacatcagcaactacctgaactggtaccagcagaagcccggcaaggcccccaaggtgctgatctacttcaccagcagcctgcacagcggcgtgcccagcaggttcagcggcagcggcagcggcaccgacttcaccctgaccatcagcagcctgcagcccgaggacttcgccacctactactgccagcagtacagcaccgtgccctggaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctga

SEQ ID NO: 7 - DNA, 2244 bp, Artificial Sequence
  transgenic construct/anti-KIR
ttaagaaaaaatacgggtagaagcagtttgagagctccgttcgccaccatggagttcggcctgtcctgggtgttcctggtggccctgttccgcggcgtgcagtgccaagtgcaactggtgcagagcggtgcagaggtgaagaagccaggatcctctgtgaaagtctcatgcaaagccagtggaggtacgttcagcttttacgcaatcagctgggtgagacaggctccgggccaaggtctggaatggatgggcggattcattccaattttcggcgcagcaaactacgcacagaagttccaggggcgggtcacgattactgctgatgagtcgacaagcacagcatacatggagctcagttctttgagaagcgatgatacagcagtttattactgtgctagaattccaagcggttcttattactatgactacgatatggatgtctggggacagggaacaacagtcaccgtatcttccgcgagcaccaagggaccttccgtgtttcccctggccccttgctccagatcaacctcagagtcgactgctgctctcggttgcttggtgaaagattattttccagaacccgttaccgtgtcttggaactccggtgcccttacttcaggggttcataccttccccgcagtcctacagtcatctggtctgtattctctcagctccgtggtcaccgtcccaagcagctcactgggcaccaagacctatacctgcaacgtcgaccataagcctagtaatacaaaggtcgacaagcgagtcgaatccaaatacggacctccttgtccaccttgccccgctcctgaattcctgggtggaccaagcgtctttctgtttccaccaaaacctaaagacacacttatgatttcccgcacacccgaagtcacctgcgtggtggtggatgtcagccaggaagaccctgaggtgcaattcaactggtacgttgatggcgtagaagtccacaacgccaagacaaaacccagagaggagcagtttaatagtacctatagggtggtgtccgtcctgaccgttctccaccaggattggttgaatggcaaggaatacaagtgtaaagtgagcaacaaaggcctaccttcgagcatagaaaagactatctccaaggcaaagggacagccaagggagccccaggtgtacacgctgccaccctctcaggaggagatgactaagaaccaggtgtccttgacctgcctcgtaaaggggttttacccctccgacatagccgtcgaatgggagagtaacggacaacccgagaacaattataaaactacacctcccgtgctcgactctgacggatccttcttcctgtactctaggctgacagtggataaatcccgttggcaggaaggaaatgtattttcctgtagtgtgatgcacgaggctctgcacaatcattatacacagaaatcactgtccttaagcctcggcaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaatcgtcctcacacagagcccggttacgctttcattgtctccaggcgagcgcgctaccctcagctgtagggcgtctcagtccgtttccagctatttagcttggtaccagcaaaaacctggccaggcccctagactgcttatttacgacgcctctaaccgagcgactggtatacccgcaaggttcagcggctcgggctcaggcaccgactttactctcaccatttcatcactggagcctgaggacttcgccgtgtattactgtcagcagaggagtaactggatgtacaccttcggccaagggaccaaattggaaatcaaaaggacagtggccgcgccaagcgtgtttatatttcccccctccgacgaacagttaaagagtggaaccgcatctgtggtatgtctgcttaacaatttttaccctcgcgaggcaaaggttcaatggaaagtcgacaacgccttacagtctggtaacagtcaagagtctgttacggagcaagacagcaaggactccacctattcgctgtcttcaacactaaccctcagcaaggctgattacgagaagcacaaagtttacgcctgtgaggtcacccatcaggggctttctagccctgtcacaaagtcgttcaacaggggagagtgctaatga

SEQ ID NO: 8 - DNA, 2232 bp, Artificial Sequence
  transgenic construct/anti-LAG-3
ttaagaaaaaatacgggtagaagcagtttgagagctccgttcgccaccatggagttcggcctgtcctgggtgttcctggtggccctgttccgcggcgtgcagtgccaggtgcaactgcagcagtggggggccggactcctgaaaccatccgaaactctctcattgacttgcgccgtatacggcgggtcattttccgattactactggaattggatccgtcagcctccaggtaaaggacttgaatggataggtgaaataaatcatcgtggaagtactaacagcaatcctagtctgaaatcaagagtaacgctgagtttggacacatctaagaaccagttttcccttaaacttagaagtgttaccgctgctgacacagctgtatactactgtgctttcggttactccgattatgagtacaattggttcgacccttggggacagggcacactagtgacagtgtccagtgcttcaaccaaggggccaagcgtgtttcccttggccccctgttcacgctccactagcgaaagcacagccgcgttaggctgtctggtgaaagactattttccagagccagtcacggtgtcttggaactcgggtgccctgacatcaggtgttcatactttccccgctgtcctccagtccagtgggctgtactcattatcttcggtggtgactgtcccctcctcatcgctggggacgaagacctatacctgtaacgtggaccacaaaccaagcaacactaaggtggacaagcgcgtggaatccaaatacgggcccccatgcccaccgtgccccgcacctgagttcctgggcggtcctagcgtctttttgttcccaccgaaaccaaaggataccctcatgatttccaggacgccggaggtcacctgcgtggtcgtggacgtgagtcaggaggaccccgaagtgcaatttaactggtatgtggacggtgtggaagtccataatgcaaagaccaagcctagagaggaacaatttaatagcacgtaccgggtggttagtgtccttaccgtcttgcaccaggattggctgaacgggaaggagtataagtgcaaggtgtcaaacaagggactgccctcatcaatcgagaagacaataagtaaggcgaagggccagccacgggagcctcaggtctacacattgccgccaagtcaagaggagatgaccaaaaaccaggtgtctttgacttgcctcgtgaagggtttttacccaagtgacatcgccgtagagtgggagtctaacggccagccagagaataactataaaacgacacctccagtgcttgattctgatggatcttttttcttgtactcaaggttgaccgtggataagagcaggtggcaagaaggcaacgtttttagttgttccgttatgcacgaggccttgcataatcactatactcagaagtccctatccctgtctctcgggcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaattgtgctgacacaatcgccagccactctgtctctgagccctggtgagcgggccactctgtcctgcagggccagtcagtctatttccagctacttggcttggtatcagcaaaaacccggccaagctcctagattgcttatttacgacgcgagcaatagggccaccggtattccagctcgctttagcggctccggaagtggtactgatttcacgctgacaatttcaagcttggaacccgaggattttgcagtgtattactgccagcagagaagcaattggccgctgaccttcggccaggggactaatcttgaaataaaacgcacggtggctgccccctccgtcttcatctttccaccttctgacgagcaattgaagagcggtaccgctagcgtagtgtgtcttctgaacaatttctacccacgcgaggccaaagtgcaatggaaagtcgacaatgctctgcagtcaggaaactcccaggagagcgtcacagagcaagattctaaagattccacttacagtctgtcatcgacattgacactgagcaaggccgattacgaaaagcataaagtgtacgcttgtgaggtcactcaccaaggactgagctcaccagttaccaaatcgtttaatagaggtgagtgttaatga

SEQ ID NO: 9 - DNA, 2250 bp, Artificial Sequence
  transgenic construct/anti-NKG2A
ttaagaaaaaatacgggtagaagcagtttgagagctccgttcgccaccatggagttcggcctgtcctgggtgttcctggtggccctgttccgcggcgtgcagtgccaggttcagttggtacagtctggcgcggaggtgaaaaaacctggagcatctgtcaaggtaagttgtaaagcttcgggctatacctttacctcatattggatgaactgggtgcggcaggcccccggtcaagggcttgaatggatgggcagaattgatccttatgattccgaaacacattacgctcagaagctgcagggcagggttaccatgacgacagacaccagcacttccactgcttacatggagctgcgatctctgcggtcagatgacacggctgtgtactattgcgccagaggcggatacgattttgacgtggggactctgtattggttctttgacgtctgggggcagggaaccactgtgacagtctcatcggcgtcaactaaagggccaagcgtgttccccttggcaccatgtagtcggtccaccagtgaaagtaccgctgcactcggttgtctggtaaaagattattttccagagcccgttactgtaagctggaacagtggggccttgacgtctggagtccatacctttccggcggtccttcagtcatctgggctgtacagcctttccagcgttgtcactgttccaagctcctccctgggcacgaagacctatacatgtaatgtcgaccataagccttcaaatacaaaagtagacaaacgtgtggaatcaaaatacggacccccatgcccgccctgtccagcgcctgaattcttgggaggcccgtctgtctttttattccccccgaaaccgaaggacaccctcatgatctcaaggacaccagaggtgacctgcgtggtggtggatgttagtcaagaggatccagaggtgcagtttaactggtatgtcgatggcgtcgaagtccacaacgctaaaaccaagccacgggaggagcagtttaactctacctatcgagtggtgtccgtactgactgtgttgcaccaggactggcttaatgggaaagaatacaagtgtaaagtttcaaacaaaggcctgccttcaagtatcgaaaagactattagcaaagctaaagggcagccaagagaaccacaggtgtacactttgcccccttcccaagaggagatgactaagaatcaagtttctctgacatgtctggtaaaaggcttctacccttctgatatagcagtggagtgggagtctaacggacagcccgaaaacaattacaagacgacaccccctgtgctagattcagacggctctttttttctttacagtcggctcacagtggataaatcacgctggcaggaagggaatgtctttagctgcagcgtcatgcatgaggctttgcacaaccattacactcagaaatctctgagcctctcacttggtaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgatatccagatgacccagagcccctccagccttagcgcttcagtcggggatagagtgacaattacctgcagagcgtctgaaaatatctacagctacttggcctggtaccaacagaaaccaggcaaagctcctaagctcctgatttataacgcaaaaaccttggctgagggggtgcccagtcgcttctcgggctctggctcaggaaccgactttacactcaccatttcttcactgcagcctgaggacttcgctacgtactactgccagcatcactatggtaccccacgcacttttggaggggggactaaggtggaaatcaagaggaccgtggccgccccatccgtatttatcttccccccatccgatgagcagttaaaatcagggaccgcctcggttgtgtgtctgcttaacaacttttacccccgcgaagctaaggtgcagtggaaggttgataacgccttgcagtctggaaacagccaggagagcgtcacagaacaagattcaaaagacagtacttattccctgagtagtacgctgaccctgtccaaagccgattacgaaaagcacaaagtatacgcgtgtgaggtcactcaccagggactatcaagcccggtcactaaatcattcaacaggggggagtgctaatga

SEQ ID NO: 10 - DNA, 2238 bp, Artificial Sequence
  transgenic construct/anti-GITR
ttaagaaaaaatacgggtagaagcagtttgagagctccgttcgccaccatggagttcggcctgtcctgggtgttcctggtggccctgttccgcggcgtgcagtgccaagtgaccttacgggaatcaggtccagcgctggtaaagcccacccaaactctaacattgacctgtactttcagcggcttttccctttcgacaagtggcatgggggtgggatggatccggcaacctcctgggaaggccctcgaatggctggcccacatttggtgggacgatgacaagtactacaacccctcactgaagtcacggctgacgatttcaaaggacaccagcaaaaatcaagtggtactgacgatgaccaatatggatcccgtcgacactgctacatactactgcgctcgcacaaggcgatactttcccttcgcctattggggacagggaactctggttaccgtgagttccgcatccactaagggcccaagcgtcttcccgctggccccttccagtaagagcacctccggagggaccgctgcacttggatgcctggttaaagactattttcctgaacctgttacagtcagttggaactcgggggcactaacctctggagtgcataccttcccagctgtactccaatcatctggactatactcactttcgtctgtcgtgactgtgccctcttcttcacttgggacccagacatatatttgcaacgtcaatcataagccatcgaacaccaaggtggataagaaggtggagccgaaatcttgtgataagacccacacatgtcctccatgcccagcccctgagctgttgggaggtccctctgtgttcctgttccccccaaaaccaaaagacaccctcatgatctccagaacgcctgaggtcacctgtgtcgttgtggacgtttctcacgaagacccagaggtcaaattcaattggtatgtcgacggggtagaggtccacaatgctaagaccaagcctcgagaagagcaatatgcttccacttacagagtggtgtctgttctgaccgtcctgcaccaagactggctaaatggaaaagagtataaatgcaaggtcagcaacaaagctctgcccgccccaattgaaaagactatcagcaaagcgaaaggccagccacgggaacctcaggtctatacgctgcccccaagccgtgatgagctcacaaagaaccaggtgagtctgacttgtctagtgaaaggcttttaccccagtgacattgcagtggagtgggagtccaacggacaacccgaaaacaactataaaactaccccccccgttctcgattctgatggcagcttcttcttatacagcaaacttacggtggataaatcgcggtggcagcagggaaatgtgttttcctgctctgtcatgcatgaggccctacataatcactatacacaaaagtctctctctttgtcccccggaaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagatagtgatgactcagtcccccgccactctctcagtaagcccaggagagagagctacgctgagctgcaaggcctcccagaacgtggggaccaacgtagcctggtaccaacaaaagcccggtcaggccccccggctgctcatttattccgcaagttatagatattcaggcattccggccagatttagtggatccggctcaggcacggagtttaccttgaccattagctctctccaatctgaggatttcgcggtgtattattgtcagcagtataacacagaccccttgaccttcggcggtggcacaaaagtcgaaattaagaggacagtcgccgccccttccgtgttcattttccccccctcagacgaacagctgaaatctggcacagcttcgtggtgtttactgaacaacttctatcccagagaagcaaaggtccagtggaaggtcgataatgccctgcagtctggtaatagtcaggagtccgtaactgagcaggacagcaaggactccacctacagcctgtcctctaccctgacactgagcaaagcagactacgaaaaacacaaagtgtacgcctgtgaggtcactcaccaggggctgagctctccagtgactaagtcttttaatcgcggcgagtgctaatga

SEQ ID NO: 11 - DNA, 2232 bp, Artificial Sequence
  transgenic construct/anti-OX40
ttaagaaaaaatacgggtagaagcagtttgagagctccgttcgccaccatggagttcggcctgtcctgggtgttcctggtggccctgttccgcggcgtgcagtgcgaagtgcagctagtgcagagtgggggcgggctagtgcagccaggggggtctcttcgcttgtcttgcgccggtagcggttttaccttttcatcgtatgcaatgtactgggtgaggcaagcccctggcaagggattggagtgggtgagcgcgatagacactgacgccggtaccttctatgcggatagtgtaagggggcgcttcaccattagtcgcgacaacgccaagaactctctttatctccagatgaattcactccgagctgaggacaccgcagtctacttttgtgctaggctgggggaaggctacttctttgattactggggacagggaaccttggtgactgtgagctctgcctccacgaaaggtccttctgttttcccccttgccccctcctcaaaatctacctctggaggtaccgccgctctaggctgcctagtaaaggattacttccctgaacccgtgacggtgtcttggaacagtggtgctcttacaagtggcgtgcacacctttcctgccgtgctccaaagttctgggctttattctctgtcctccgtggtgaccgtgcccagcagctctcttggtacccagacttacatctgtaatgttaaccacaaaccctcaaatacaaaggtggacaagagagtggagccaaagagctgcgataaaacccacacctgtcccccctgtcccgcacctgagcttcttgggggccctagtgtgtttctgttcccacctaagcccaaagacaccctgatgatctctaggaccccggaggttacctgcgtagttgtggacgtctcccacgaggatcccgaggtgaaattcaattggtacgtcgacggcgttgaagtccataacgccaagaccaagcctcgcgaagaacagtataactctacctatcgcgtggtgtcggtcctgactgtgcttcaccaggactggcttaacgggaaggaatataaatgcaaggtcagcaataaggccctccccgctccaattgagaagaccatttccaaagctaaaggccagccacgggagccccaggtctacactctaccaccctcccgcgaggagatgaccaaaaaccaggtgtccctgacctgtttggtgaagggattttacccgtccgatatcgctgtggagtgggaaagtaatggccaacctgaaaacaactataagacaaccccgccagtcctagatagcgatggatccttcttcctgtatagtaaacttacagtggataaaagtaggtggcagcagggtaacgttttttcgtgtagtgtgatgcatgaagcactccacaatcactatacccaaaaaagcctttcgctgtcccctggccgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagattgtgctgacgcagagcccagcaactttgagtctctcgcccggcgagcgcgccacactttcctgtcgagcctcccaatccgtgagcagttacctggcatggtaccaacagaagcctgggcaagcccccagattgctgatttatgatgcctccaatcgagcaacaggaataccagctcggttcagtgggagcggaagcggtacagatttcactctgacaatcagctcgctggagccagaggacttcgctgtttattactgccagcagcgttcgaactggcccccaacgtttggaggaggcaccaaagtggaaataaaacgaacagtagcagccccatctgtcttcatcttcccccccagcgatgaacagctgaagtcaggcactgcttccgtcgtctgcctactgaacaatttttatcctagagaggccaaggtccaatggaaggtagacaacgcactccaatcagggaactcacaagagtctgtcaccgagcaggactctaaggactcgacatactcattatctagtaccctgacactgagcaaggccgactacgaaaaacataaagtctacgcctgtgaagtgacgcaccagggactcagctctcccgtcactaagagcttcaatcggggggagtgttaatga

SEQ ID NO: 12 - DNA, 924 bp, Artificial Sequence
  transgenic construct/CD80
ttaagaaaaaatacgggtagaagtggctttcacttttgaccctaagcatctgccaccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 13 - DNA, 1626 bp, Artificial Sequence
  transgenic construct/(ns)hIL-12
ttaagaaaaaatacgggtagaagcagtttgagagctccgttcgccaccatgagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggctccggcgagggccgcggctccctgctgacctgcggcgacgtggaggagaaccccggccccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaa

SEQ ID NO: 14 - DNA, 768 bp, Artificial Sequence
  transgenic construct/eGFP
ttaagaaaaaatacgggtagaattggagcggccgcattgtgccaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaga

SEQ ID NO: 15 - DNA, 2214 bp, Artificial Sequence
  transgenic construct/Nivolumab
ttaagaaaaaatacgggtagaagcagtttgagagctccgttcgccaccatggagttcggcctgtcctgggtgttcctggtggccctgtttagaggcgtgcagtgccaggtgcagctggtggagagcggaggaggagtggtgcagccaggccggagcctgcgcctggactgtaaggccagcggcatcacctttagcaactccggaatgcactgggtgaggcaggcaccaggcaagggcctggagtgggtggccgtgatctggtacgacggctctaagcggtactatgccgatagcgtgaagggccggttcacaatctccagagacaactctaagaataccctgttcctccagatgaacagcctgagggccgaggatacagccgtgtactattgcgccaccaatgacgattactggggccagggcacactggtgaccgtgagcagcgccagcaccaagggaccaagcgtgttcccactggcaccttgctctcgcagcacatccgagtctaccgccgccctgggatgtctggtgaaggattatttccccgagcctgtgaccgtgagctggaacagcggcgccctgacatccggagtgcacacctttcccgccgtgctccagagcagcggcctgtacagcctgtcctctgtggtgacagtgcccagctcctctctgggcaccaagacatatacctgcaacgtggaccacaagcctagcaataccaaggtggataagcgggtggagtccaagtacggaccaccttgcccaccatgtccagcaccagagttcctgggaggaccaagcgtgttcctgtttcctccaaagcccaaggacaccctgatgatctccagaacaccagaggtgacctgcgtggtggtggacgtgtctcaggaggaccccgaggtgcagttcaactggtatgtggatggcgtggaggtgcacaatgccaagaccaagcccagggaggagcagtttaactccacataccgcgtggtgtctgtgctgaccgtgctgcaccaggactggctgaacggcaaggagtataagtgcaaggtgtccaataagggcctgcctagctccatcgagaagacaatcagcaaggcaaagggacagccaagggagccacaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtattccaggctgacagtggataagtctcgctggcaggagggcaacgtgtttagctgctccgtgatgcacgaggccctgcacaatcactacacccagaagtctctgagcctgtccctgggcaagcggagaaagaggggcagcggagagggaaggggctccctgctgacatgtggcgacgtggaggagaatcctggcccaatgaagtatctgctgcccaccgccgccgccggcctgctgctgctggcagcacagcctgctatggccgagatcgtgctgacacagtccccagccaccctgtctctgagccctggagagagggccaccctgagctgtagagcatcccagagcgtgagcagctacctggcctggtatcagcagaagcctggacaggcaccaaggctgctgatctacgatgcaagcaacagggcaacaggcatccccgcaagattcagcggctccggctctggcaccgactttacactgaccatctcctctctggagcctgaggatttcgccgtgtactattgccagcagagcagcaattggccacggacatttggccagggcaccaaggtggagatcaagagaacagtggccgcccctagcgtgttcatctttcctccatccgacgagcagctgaagagcggcaccgcatccgtggtgtgcctgctgaacaatttctacccacgggaggccaaggtgcagtggaaggtggataacgccctccagtccggcaattctcaggagagcgtgacagagcaggactccaaggattctacctatagcctgtctagcacactgaccctgtccaaggccgactacgagaagcacaaggtgtatgcctgcgaggtgacacaccagggcctgtcctctcccgtgaccaagagctttaacagaggcgagtgttgatga

SEQ ID NO: 16 - DNA, 2238 bp, Artificial Sequence
  transgenic construct/anti-CTLA-4
ttaagaaaaaatacgggtagaagcagtttgagagctccgttcgccaccatggagtttgggctgtcatgggtctttctggtcgctctgtttaggggggtgcagtgtcaggtgcagctggtcgagtcaggggggggcgtggtgcagccaggcaggtccctgaggctgtcttgcgcagccagcggcttcacctttagctcctacacaatgcactgggtgcggcaggcacctggcaagggcctggagtgggtgaccttcatctcctatgacggcaacaataagtactatgccgattctgtgaagggccggtttaccatctccagagacaactctaagaatacactgtacctgcagatgaacagcctgagggccgaggacaccgccatctactattgcgcaaggacaggatggctgggacctttcgattattggggccagggcaccctggtgacagtgtctagcgcctctaccaagggcccaagcgtgtttcccctggccccttcctctaagtccacctctggaggcacagccgccctgggctgtctggtgaaggattacttccctgagccagtgaccgtgagctggaactccggcgccctgaccagcggagtgcacacatttccagccgtgctgcagagcagcggcctgtactccctgtctagcgtggtgaccgtgccctcctctagcctgggcacccagacatatatctgcaacgtgaatcacaagcctagcaatacaaaggtggacaagcgggtggagccaaagtcctgtgataagacccacacatgcccaccttgtccagcacctgagctgctgggaggaccatccgtgttcctgtttccacccaagcccaaggacaccctgatgatcagccggacccctgaggtgacatgcgtggtggtggacgtgagccacgaggaccccgaggtgaagttcaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctcccagccgggacgagctgaccaagaaccaggtgagcctgacatgtctggtgaagggcttctatcccagcgacatcgccgtggagtgggagtccaatggccagcctgagaacaattacaagaccacaccccctgtgctggactctgatggcagcttctttctgtattctaagctgaccgtggacaagagcagatggcagcagggcaacgtgtttagctgctccgtgatgcacgaggccctgcacaatcactacacacagaagtctctgagcctgtcccctggcaagcggagaaagaggggcagcggagagggaaggggctccctgctgacctgtggcgatgtggaggagaatcctggcccaatgaagtatctgctgccaacagctgccgccggcctgctgctgctggccgcacagcccgctatggccgagatcgtgctgacccagagcccaggcacactgtctctgagccctggagagagggccaccctgtcctgcagagcctcccagtctgtgggctcctcttacctggcctggtatcagcagaagcctggccaggccccaaggctgctgatctacggagccttcagcagggccaccggcatccccgaccgcttcagcggctccggctctggcacagacttcaccctgacaatctccaggctggagcccgaggacttcgccgtgtactattgccagcagtacggcagctccccttggacctttggccagggcacaaaggtggagatcaagaggaccgtggcagcaccatccgtgttcatctttccaccctctgacgagcagctgaagagcggcacagcctccgtggtgtgcctgctgaacaacttctacccccgcgaggccaaggtgcagtggaaggtggataacgccctgcagtccggcaattctcaggagagcgtgaccgagcaggactccaaggattctacatatagcctgtctagcaccctgacactgagcaaggccgattacgagaagcacaaggtgtatgcctgtgaggtcacccaccagggactgtcatcacccgtcaccaaaagtttcaatagaggagagtgctga

SEQ ID NO: 17 - DNA, 1662 bp, Artificial Sequence
  transgenic construct/IL-12
ttaagaaaaaatacgggtagaagcagtttgagagctccgttcgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatga

SEQ ID NO: 18 - DNA, 708 bp, Artificial Sequence
  transgenic construct/NS1
ttaagaaaaaatacgggtagaagcagtttgagagctccgttcgccaccatggatagcaataccatgtcctcctttcaggttgactgtttcctgtggcatattcgcaagcgtttcgccgataatggtctgggtgacgccccgttcctggaccgtctgcgtcgcgatcagaagagcctgaaaggccgcggtaacaccctgggcctggacatcgaaaccgcgaccctggtgggcaagcagatcgtggagtggattctgaaagaggaaagcagcgaaaccctgcgtatgaccattgccagcgtgccgaccagccgttacctgagcgacatgaccctggaggaaatgagccgtgattggttcatgctgatgccgcgccagaagatcattggtccactgtgcgtgcgtctggaccaggctatcatggagaagaacattgtgctgaaagccaacttcagcgtgatctttaaccgtctggaaaccctgattctgctgcgcgcctttaccgaggaaggcgcgatcgtgggcgagattagcccgctgccgagcctgccaggtcacacctatgaagatgtgaaaaatgccgtgggcgtgctgattggcggtctggagtggaacggtaacaccgtgcgtgtgagcgaaaacattcagcgcttcgcatggcgtaactgtgatgagaatggtcgtccaagcctgccaccggaacaaaaataa

SEQ ID NO: 19 - DNA, 414 bp, Artificial Sequence
  transgenic construct/Apoptin
ttaagaaaaaatacgggtagaagcagtttgagagctccgttcgccaccatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaa

SEQ ID NO: 20 - DNA, 1104 bp, Artificial Sequence
  transgenic construct/B18R
ttaagaaaaaatacgggtagaatacaattaacgatctttatagccaccatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaa

SEQ ID NO: 21 - DNA, 2232 bp, Artificial Sequence
  transgenic construct/anti-CD28
ttaagaaaaaatacgggtagaagcagtttgagagctccgttcgccaccatggagtttgggctgagttgggtctttctggtcgctctgttccggggggtgcagtgtcaggtgcagctggtgcagtctggggccgaggtcaagaagcccggcgcctccgtgaaggtgtcttgcaaggccagcggctacaccttcacaagctactatatccactgggtgaggcaggcaccaggacagggcctggagtggatcggctgttaccctggcaacgtgaataccaactataatgagaagtttaaggaccgcgccacaatcctgacctgggatacaagctccaccgcctatatggagctgtccaggctgcgctctgacgatacagccgtgtacttctgcaccaggtcccactatggcctgcagtggaactttgacgtgtggggccagggcaccacagtgacagtgtctagcgcctctaccaagggccctagcgtgttccctctggccccatgctcccgctctacaagcgagtccaccgccgccctgggatgtctggtgaaggattacttccctgagccagtgaccgtgagctggaattccggcgccctgacaagcggagtgcacacctttccagccgtgctgcagtcctctggcctgtactccctgagctccgtggtgacagtgccttctagctccctgggcaccaagacatatacctgcaacgtggaccacaagccaagcaataccaaggtggataagcgggtggagagcaagtacggaccaccttgcccatcctgtccagcacctgagtttctgggaggaccttccgtgttcctgtttccacccaagccaaaggacacactgatgatctctagaacacccgaggtgacctgcgtggtggtggacgtgagccaggaggatcctgaggtgcagttcaactggtacgtggatggcgtggaggtgcacaatgccaagaccaagccacgggaggagcagtttaactccacatacagagtggtgtctgtgctgaccgtgctgcaccaggactggctgaacggcaaggagtataagtgcaaggtgtccaataagggcctgccctctagcatcgagaagacaatctctaaggcaaagggacagccaagggagcctcaggtgtacaccctgcctccaagccaggaggagatgacaaagaaccagtggtccctgacctgtctggtgaagggcttctatccttctgacatcgccgtggagtgggagagcaatggccagccagagaacaattacaagaccacaccccctgtgctggactctgatggcagcttctttctgtattctaggctgacagtggataagagccgctggcaggagggcaacgtgttttcttgcagcgtgatgcacgaggccctgcacaatcactacacccagaagtccctgtctctgagcctgggcaagcggagaaagaggggaagcggagagggaagaggctccctgctgacatgtggcgatgtggaggagaacccaggccccatgaagtatctgctgcccaccgcagccgccggcctgctgctgctggcagcccagccagccatggccgacatccagatgacccagagcccatcctctctgtccgcctctgtgggcgatagggtgacaatcacctgccacgcctcccagaacatctacgtgtggctgaattggtatcagcagaagccaggcaaggcccccaagctgctgatctacaaggcctccaatctgcacacaggagtgccttctcgcttcagcggatccggatctggaaccgactttacactgaccatcagctccctgcagccagaggatttcgccacctactattgccagcagggccagacatacccctatacctttggcggcggcacaaaggtggagatcaagaggaccgtggcagcaccatccgtgttcatctttccaccctctgacgagcagctgaagagcggaaccgcatccgtggtgtgcctgctgaacaatttctaccctagagaggccaaggtgcagtggaaggtggataacgccctgcagtccggcaattctcaggagagcgtgacagagcaggactccaaggattctacctatagcctgtctagcacactgaccctgagcaaggccgactacgagaagcacaaggtgtatgcctgtgaagtcacccaccaggggctgagttcaccttggacaaagagtttcaatcggggagagtgttgataa

SEQ ID NO: 22 - DNA, 2256 bp, Artificial Sequence
  transgenic construct/anti-CA 15-3
ttaagaaaaaatacgggtagaagcagtttgagagctccgttcgccaccatggagttcggcctgagttgggtcttcctggtcgccctgtttcggggggtccagtgtgaggtccagctgcagcagtccgggggagagagagccacccccggcgcctccgtgaagatgtcttgcaagacaagcggctacaccttcacaaactattggatgcactgggtgaagcagcggcccggacagggcctggagtggatcatcggctacatcaaccctagctccggctacacccagtataatcagaagtttaaggacaaggccaccctgacagccgataagtctagctccaccgcctatatccagctgtctagcctgacatctgaggacagcgccgtgtactattgctccacctactatggcgattacctgttcccatattggggacagggcaccctggtgacagtgtccgccgcatctacaaagggaccaagcgtgtttccactggcaccttcctctaagtctaccagcggaggaacagccgccctgggatgtctggtgaaggactacttccctgagccagtgaccgtgagctggaactccggcgccctgacctccggagtgcacacatttcctgccgtgctgcagagctccggcctgtacagcctgtctagcgtggtgaccgtgccatcctctagcctgggcacccagacatatatctgcaacgtgaatcacaagccttccaatacaaaggtggacaagcgggtggagccaaagtcttgtgataagacccacacatgcccaccttgtccagcacctgagctgctgggaggaccaagcgtgttcctgtttccacccaagcccaaggacaccctgatgatcagcagaacccccgaggtgacatgcgtggtggtggacgtgtcccacgaggatcctgaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatccaagcgacatcgccgtggagtgggagtccaatggccagcccgagaacaattacaagaccacaccccctgtgctggactctgatggcagcttctttctgtattctaagctgaccgtggataagagccggtggcagcagggcaacgtgttttcctgctctgtgatgcacgaggccctgcacaatcactacacacagaagagcctgtccctgtctcccggcaagcggagaaagaggggaagcggagagggaagaggctccctgctgacctgtggcgacgtggaggagaaccctggcccaatgaagtatctgctgcctacagcagccgccggcctgctgctgctggcagcacagccagccatggccgatgtgctgatgacccagacaccactgtccctgccagtgtctctgggcgaccaggccagcatctcctgcaggtcctctcaggacatcgtgtacggcaacggcaatacctacctggagtggtatctgcagaagcccggccagtcccctaagctgctgatctataaggtgagcaataggttctccggcgtgccagaccgcttttctggcagcggctccggcaccgatttcacactgaagatctccagggtggaggcagaggacctgggcgtgtactattgcttccagggcagccacgtgccctacacctttggcggcggcacaaagctggagatcaagaggaggaccgtggcagcaccttccgtgttcatctttccaccctctgacgagcagctgaagtctggcacagccagcgtggtgtgcctgctgaacaacttctaccccagggaggccaaggtgcagtggaaggtggataacgccctgcagtccggcaattctcaggagagcgtgaccgagcaggactccaaggattctacatatagcctgagctccaccctgacactgagcaaggccgattacgagaagcacaaggtgtatgcctgtgaggtcacccaccagggactgagtagccccgtcaccaaatccttcaatagaggcgagtgttga

SEQ ID NO: 23 - DNA, 2262 bp, Artificial Sequence
  transgenic construct/ anti-CA19-9
ttaagaaaaaatacgggtagaagcagtttgagagctccgttcgccaccatggagtttgggctgtcatgggtctttctggtcgctctgtttcggggggtgcagtgtcaggtgcagctggtcgagtcaggggggggaagtgtgcagcctggccggagcctgagactgagctgcgaggcatccggcttcacctttgaggcatacgcaatgcactgggtgcggcagccacctggcaagggcctggagtgggtgagctccatcaactggaattccggccggatcgcctacgccgactctgtgaagggcagattcaccatcagcagggataacgcccgcaattccctgtatctgcagatgaactctctgagactggaggacaccgccttttactattgcgccaaggacatccggagattcagcacaggcggcgccgagtttgagtactggggccagggcaccctggtgacagtgtctagcgccagcaccaagggaccatccgtgttccctctggcaccatcctctaagagcacctccggaggaacagccgccctgggctgtctggtgaaggactatttcccagagcccgtgacagtgtcctggaactctggcgccctgaccagcggagtgcacacatttcctgccgtgctgcagagctccggcctgtactccctgtctagcgtggtgaccgtgccatcctctagcctgggcacccagacatatatctgcaacgtgaatcacaagccatccaatacaaaggtggacaagagggtggagcccaagtcttgtgataagacccacacatgcccaccctgtcctgcaccagagctgctgggaggaccaagcgtgttcctgtttcctccaaagcctaaggacaccctgatgatcagccgcaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtataagtgcaaggtgtctaataaggccctgcccgcccctatcgagaagaccatctccaaggcaaagggacagccaagggagcctcaggtgtacacactgcccccttctcgcgacgagctgaccaagaaccaggtgagcctgacatgtctggtgaagggcttctatcctagcgacatcgccgtggagtgggagtccaatggccagccagagaacaattacaagaccacaccacccgtgctggacagcgatggctccttctttctgtatagcaagctgaccgtggacaagtccaggtggcagcagggcaacgtgttttcttgcagcgtgatgcacgaggccctgcacaatcactacacacagaagtccctgtctctgagcccaggcaagaggaggaagaggggatccggagagggaagaggctctctgctgacctgtggcgatgtggaggagaatccaggccccatgaagtatctgctgcccacagcagccgccggcctgctgctgctggcagcacagcctgccatggcccagagcgtgctgacccagcctccatccgcctctggaaccccaggacagagggtgacaatcagctgctccggctcctctagcaacatcggcagcaatttcgtgtactggtatcagcagctgccaggaaccgcaccaaagctgctgatctacaggaacaatcagcggccctccggagtgccagacaggttttctggcagccgctccggcacatctgccagcctggccatctctggcctgcggagcgaggacgaggcagattactattgcgcagcatgggacgatagcctgggaggacactacgtgttcggaaccggaacaaaggtgaccgtgctgagaacagtggcagcaccaagcgtgttcatctttccccctagcgacgagcagctgaagtccggcaccgcctctgtggtgtgcctgctgaacaacttctaccctagagaggccaaggtgcagtggaaggtggataacgccctgcagtctggcaatagccaggagtccgtgaccgagcaggactctaaggatagcacatattccctgtcctctaccctgacactgtccaaggccgattacgagaagcacaaggtgtatgcctgtgaagtgacccaccagggactgagcagccccgtgaccaaatctttcaaccgaggcgaatgttga

SEQ ID NO: 24 - DNA, 2232 bp, Artificial Sequence
  transgenic construct/anti-CA 125
ttaagaaaaaatacgggtagaagcagtttgagagctccgttcgccaccatggagtttgggctgtcctgggtgttcctggtcgctctgtttaggggggtgcagtgtgaagtgcagctggtcgtcgagtccggggggggcctggtgcagccaggaggcagcctgaggctgtcctgcgcagcatccggctactctatcaccaacgactatgcctggaattgggtgagacaggcaccaggcaagggcctggagtgggtgggctacatctcttatagcggctacaccacatataaccccagcctgaagtccaggttcaccatctctcgcgataccagcaagaacacactgtacctgcagatgaattccctgagggcagaggacaccgccgtgtactattgcgccagatggacaagcggcctggattattggggccagggcaccctggtgacagtgagctccgccagcaccaagggcccttccgtgtttcccctggccccttctagcaagtccacctctggaggaacagccgccctgggctgtctggtgaaggactacttccctgagccagtgaccgtgtcttggaacagcggcgccctgacctccggagtgcacacatttcccgccgtgctgcagtcctctggcctgtacagcctgagctccgtggtgaccgtgccttctagctccctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatcagcagaaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatcccagcgacatcgccgtggagtgggagtccaatggccagcctgagaacaattacaagaccacaccccctgtgctggactccgatggctctttctttctgtatagcaagctgaccgtggataagtcccggtggcagcagggcaacgtgtttagctgctccgtgatgcacgaggccctgcacaatcactacacacagaagtctctgagcctgtccccaggcaagaggagaaagaggggatccggcgagggcagaggctctctgctgacctgtggcgacgtggaggagaaccctggcccaatgaagtatctgctgcctacagcagccgccggcctgctgctgctggcagcacagcctgccatggccgacatccagatgacccagtccccatctagcctgtctgccagcgtgggcgacagggtgaccatcacatgcaaggccagcgatctgatccacaattggctggcctggtaccagcagaagcctggcaaggccccaaagctgctgatctatggagccacctctctggagacaggagtgccaagccgcttctccggatctggaagcggcaccgactttaccctgacaatctcctctctgcagcctgaggatttcgccacatactattgccagcagtactggaccacaccattcacctttggccagggcacaaaggtggagatcaagaggaccgtggccgccccttccgtgttcatctttccaccctctgacgagcagctgaagtccggcacagcctctgtggtgtgcctgctgaacaacttctacccacgcgaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgtgaagtcacccaccaggggctgtcatcaccagtcactaagagtttcaatcggggggaatgctga

SEQ ID NO: 25 - DNA, 2238 bp, Artificial Sequence
  transgenic construct/anti-EGFR
ttaagaaaaaatacgggtagaagcagtttgagagctccgttcgccaccatggagttcgggctgagttgggtgttcctggtcgctctgtttcggggggtgcagtgtcaggtgcagctgaagcagtctgggcctgggctggtccagccctctcagagcctgtccatcacctgcacagtgtctggcttcagcctgaccaattacggagtgcactgggtgcggcagagccctggcaagggcctggagtggctgggcgtgatctggtccggaggcaatacagactataacacccccttcacctccagactgtctatcaataaggataactctaagagccaggtgttctttaagatgaatagcctgcagtccaacgacaccgccatctactattgcgccagggccctgacatactatgattacgagttcgcctattggggccagggcaccctggtgacagtgtctgccgccagcaccaagggccctagcgtgtttcccctggcccctagctccaagtccacctctggaggaacagccgccctgggctgtctggtgaaggactacttccctgagccagtgaccgtgtcctggaattctggcgccctgaccagcggcgtgcacacatttcccgccgtgctgcagtctagcggcctgtactccctgtcctctgtggtgaccgtgcctagctcctctctgggcacccagacatatatctgcaatgtgaaccacaagccatccaacacaaaggtggacaagaaggtggagcccaagtcttgtgataagacccacacatgcccaccttgtccagcacctgagctgctgggaggaccatccgtgttcctgtttccacccaagcctaaggacaccctgatgatctctagaaccccagaggtgacatgcgtggtggtggacgtgtcccacgaggatcccgaggtgaagttcaattggtacgtggatggcgtggaggtgcacaacgccaagaccaagccacgggaggagcagtacaactccacctatagagtggtgtctgtgctgacagtgctgcaccaggactggctgaatggcaaggagtacaagtgcaaggtgagcaacaaggccctgccagcccccatcgagaagaccatctccaaggcaaagggacagccaagggagccacaggtgtacacactgcctccatctcgcgacgagctgaccaagaatcaggtgagcctgacatgtctggtgaagggcttctatcctagcgacatcgcagtggagtgggagtccaacggacagccagagaacaattacaagaccacaccccctgtgctggacagcgatggctccttctttctgtattctaagctgaccgtggataagagccggtggcagcagggcaacgtgttcagctgctccgtgatgcacgaggccctgcacaaccactacacacagaagtctctgagcctgtcccctggcaagcggagaaagaggggaagcggagagggaaggggctccctgctgacctgtggcgacgtggaggagaatcctggcccaatgaagtatctgctgcccacagcagccgccggcctgctgctgctggcagcacagcctgccatggccgacatcctgctgacccagagccctgtgatcctgtctgtgagcccaggcgagcgggtgtccttctcttgcagagccagccagtccatcggcaccaatatccactggtaccagcagaggacaaacggcagccctcgcctgctgatcaagtatgcctctgagagcatctccggcatcccatccagattctctggcagcggctccggcaccgactttacactgtctatcaacagcgtggagtccgaggacatcgccgattactattgccagcagaacaataactggccaaccacattcggcgccggcaccaagctggagctgaagaggacagtggccgccccctccgtgttcatctttccaccctctgacgagcagctgaagtctggcaccgccagcgtggtgtgcctgctgaataacttttacccccgcgaggccaaggtgcagtggaaggtggataatgccctgcagtctggcaacagccaggagtccgtgaccgagcaggactctaaggatagcacatattccctgagctccaccctgacactgagcaaggccgattacgagaagcacaaggtgtatgcctgtgaagtcacccaccaggggctgtcctcacccgtcacaaaatctttcaataggggggagtgttga

SEQ ID NO: 26 - DNA, 2244 bp, Artificial Sequence
  transgenic construct/anti-HER2
ttaagaaaaaatacgggtagaagcagtttgagagctccgttcgccaccatggagttcgggctgagttgggtgttcctggtcgctctgtttaggggagtgcagtgtcaggtgcagctggtcgagtctgggggggggctggtccagcctggaggctctctgaggctgagctgcgcagcatccggctttaacatcaaggacacctacatccactgggtgcggcaggcaccaggcaagggcctggagtgggtggccagaatctatcctaccaatggctacacacggtatgccgacagcgtgaagggcagattcaccatctctgccgataccagcaagaacacagcctacctgcagatgaattccctgcgggccgaggatacagccgtgtactattgctctagatggggcggcgacggcttttacgccatggattattggggccagggcaccctggtgacagtgagctccgcctctaccaagggaccaagcgtgttcccactggcaccttctagcaagtctaccagcggaggaacagccgccctgggatgtctggtgaaggactactttcctgagccagtgaccgtgtcctggaactctggcgccctgaccagcggagtgcacacattcccagccgtgctgcagtcctctggcctgtactccctgagctccgtggtgaccgtgccctctagctccctgggcacccagacatatatctgcaacgtgaatcacaagcctagcaataccaaggtggacaagaaggtggagccaaagtcctgtgatacaccacctccatgcccaaggtgtccagcacctgagctgctgggaggaccaagcgtgttcctgtttccacctaagcctaaggacaccctgatgatctctcgcacccctgaggtgacatgcgtggtggtggacgtgtcccacgaggatccagaggtgaagtttaactggtacgtggatggcgtggaggtgcacaatgccaagaccaagcccagggaggagcagtacaactccacctatcgcgtggtgtctgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgccaccctctcgcgacgagctgaccaagaaccaggtgagcctgacatgtctggtgaagggcttttatccaagcgacatcgccgtggagtgggagtccaatggccagcccgagaacaattacaagaccacacctccagtgctggacagcgatggctccttctttctgtattctaagctgaccgtggataagagccggtggcagcagggcaacgtgttctcctgctctgtgatgcacgaggccctgcacaatcactacacacagaagagcctgtccctgtctcccggcaagcggagaaagaggggaagcggagagggaagaggctccctgctgacctgtggcgacgtggaggagaaccctggcccaatgaagtatctgctgcctacagcagccgccggcctgctgctgctggcagcacagcctgccatggccgacatccagatgacccagagcccatctagcctgagcgcctccgtgggcgacagggtgaccatcacatgccgcgcctcccaggatgtgaatacagccgtggcctggtaccagcagaagcctggcaaggccccaaagctgctgatctactctgccagcttcctgtattccggcgtgccatctaggttttccggctctcgcagcggaaccgacttcaccctgacaatctcctctctgcagcccgaggattttgccacatactattgccagcagcactacaccacaccccctaccttcggccagggcacaaaggtggagatcaagaggaccgtggcagcaccttccgtgttcatctttccaccctctgacgagcagctgaagagcggcacagcctccgtggtgtgcctgctgaacaatttctacccaagagaggccaaggtgcagtggaaggtggataacgccctgcagtctggcaatagccaggagtccgtgaccgagcaggactctaaggatagcacatattccctgagctccaccctgacactgagcaaggccgactacgagaagcacaaggtgtatgcctgtgaagtcacccaccaggggctgagcagtcccgtcacaaaatcctttaacagaggcgaatgttgataa

SEQ ID NO: 27 - DNA, 1458 bp, Artificial Sequence
  transgenic construct/anti-nfP2X7
ttaagaaaaaatacgggtagaagcagtttgagagctccgttcgccaccatggagttcgggctgagttgggtgttcctggtcgctctgtttaggggggtgcagtgtgaagtgcagctgctggagtccggggggggcctggtccagccaggaggcagcctgaggctgtcctgcgcagcatctggcttcacctttaggaaccacgacatgggatgggtgcgccaggcacctggcaagggcctggagtgggtgagcgccatctctggaagcggaggatccacatactatgccaactctgtgaagggccggttcaccatcagcagagacaactccaagaatacactgtacctgcagatgaatagcctgcgggccgaggataccgccgtgtactattgcgccgagccaaagcccatggacacagagttcgattatagatccccaggcaccctggtgacagtgagctccgcctctaccaagggccccagcgtgtttcctctggccccatctagcaagtccacctctggaggaacagccgccctgggctgtctggtgaaggattacttccctgagccagtgacagtgtcttggaacagcggcgccctgacctctggagtgcacacatttcccgccgtgctgcagtcctctggcctgtactccctgagctccgtggtgaccgtgccttctagctccctgggcacccagacatatatctgcaacgtgaatcacaagccttctaatacaaaggtggacaagagggtggagccaaagagctgtgataagacccacacatgcccaccttgtccagcacctgagctgctgggaggaccaagcgtgttcctgtttccacccaagcccaaggacaccctgatgatctcccgcacccctgaggtgacatgcgtggtggtggacgtgtctcacgaggatccagaggtgaagtttaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgagcaataaggccctgccagcccccatcgagaagaccatctccaaggcaaagggacagccaagggagcctcaggtgtacacactgcctccaagccgcgacgagctgaccaagaaccaggtgtccctgacatgtctggtgaagggcttctatccctccgacatcgccgtggagtgggagtctaatggccagcctgagaacaattacaagaccacaccccctgtgctggactccgatggctctttctttctgtattctaagctgaccgtggataagagcagatggcagcagggcaacgtgttcagctgttccgtgatgcacgaagcactgcacaaccactacactcagaaatccctgtcactgtcacctggcaagtgataa

SEQ ID NO: 28 - DNA, 2232 bp, Artificial Sequence
  transgenic construct/anti-PSMA
ttaagaaaaaatacgggtagaagcagtttgagagctccgttcgccaccatggagtttggactgagttgggtgttcctggtcgccctgtttagaggcgtgcagtgtgaagtgcagctggtgcagagcggccctgaagtgaaaaagccaggcgccaccgtgaagatctcttgcaagacaagcggctacaccttcacagagtataccatccactgggtgaagcaggcaccaggcaagggcctggagtggatcggcaacatcaatcctaacaatggcggcaccacatacaaccagaagtttgaggacaaggccaccctgacagtggacaagagcaccgatacagcctatatggagctgagctccctgcggtccgaggacaccgccgtgtactattgcgccgccggctggaatttcgattactggggccagggcaccctgctgacagtgtctagcgccagcacaaagggaccatccgtgtttccactggcaccttcctctaagtccacctctggaggaacagccgccctgggctgtctggtgaaggattacttccctgagccagtgaccgtgtcctggaactctggcgccctgacctccggagtgcacacatttccagccgtgctgcagagctccggcctgtacagcctgtctagcgtggtgaccgtgccttcctctagcctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatctctagaacccctgaggtgacatgcgtggtggtggacgtgagccacgaggatccagaggtgaagttcaactggtacgtggatggcgtggaggtgcacaatgccaagaccaagcctagggaggagcagtacaactctacctatcgcgtggtgagcgtgctgacagtgctgcaccaggactggctgaacggcaaggagtataagtgcaaggtgtccaataaggccctgccagcccccatcgagaagaccatcagcaaggcaaagggacagccaagggagccacaggtgtacacactgcctccatctagagacgagctgaccaagaaccaggtgagcctgacatgtctggtgaagggcttctatccatccgacatcgccgtggagtgggagtctaatggccagcccgagaacaattacaagaccacaccccctgtgctggacagcgatggctccttctttctgtatagcaagctgaccgtggacaagtccaggtggcagcagggcaacgtgtttagctgctccgtgatgcacgaggccctgcacaatcactacacacagaagtctctgagcctgtccccaggcaagaggagaaagaggggatccggagagggaaggggctctctgctgacctgtggcgatgtggaggagaaccctggcccaatgaagtatctgctgcctacagcagccgccggcctgctgctgctggcagcacagcctgccatggccgacatccagatgacccagtccccatcctctctgtctacaagcgtgggcgatcgcgtgaccctgacatgcaaggcatcccaggacgtgggaaccgcagtggattggtaccagcagaagcccggccctagcccaaagctgctgatctattgtgccagcacccggcacacaggcatcccatccagattctccggctctggcagcggcaccgactttaccctgacaatcatcagctccctgcagccagaggacttcgcagcagattactattgccagcagtacaatagctatcctctgacctttggcccaggcacaaaggtggacatcaagaggaccgtggccgcccctagcgtgttcatctttccacccagcgatgagcagctgaagtccggcacagcctctgtggtgtgcctgctgaacaacttctacccacgcgaggccaaggtgcagtggaaggtggacaacgccctgcagtctggcaatagccaggagtccgtgaccgagcaggactctaaggatagcacatattccctgtctagcaccctgacactgagcaaggccgattacgagaagcacaaggtgtatgcctgcgaggtcactcatcaggggctgtcatcaccagtcaccaaaagtttcaataggggggaatgttga

SEQ ID NO: 29 - DNA, 2232 bp, Artificial Sequence
  transgenic construct/anti-VEGFR
ttaagaaaaaatacgggtagaagcagtttgagagctccgttcgccaccatggagttcggcctgtcttgggtgttcctggtcgccctgtttaggggagtgcagtgtgaagtgcagctggtgcagtctggggggggcctggtgaagccaggaggcagcctgaggctgtcctgcgcagcatctggcttcacctttagctcctacagcatgaactgggtgcgccaggcaccaggcaagggcctggagtgggtgtctagcatctcctctagctcctcttacatctactatgccgactccgtgaagggcaggttcaccatctctcgcgataacgccaagaatagcctgtatctgcagatgaattccctgagggccgaggacaccgccgtgtactattgcgcccgcgtgacagacgccttcgacatctggggccagggcaccatggtgacagtgagctccgcctctaccaagggaccaagcgtgctgccactggcaccatctagcaagtctaccagcggaggaacagccgccctgggatgtctggtgaaggattacttccccgagcctgtgacagtgtcttggaacagcggcgccctgaccagcggagtgcacacatttcccgccgtgctgcagtcctctggcctgtactccctgagctccgtggtgaccgtgccttctagctccctgggcacccagacatatatctgcaacgtgaatcacaagcctagcaatacaaaggtggacaagagggtggagccaaagtcctgtgataagacccacacatgcccaccttgtccagcaccagagctgctgggaggaccaagcgtgttcctgtttccacccaagcccaaggacaccctgatgatctctcgcacccctgaggtgacatgcgtggtggtggacgtgtcccacgaggatccagaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtataagtgcaaggtgagcaataaggccctgcctgccccaatcgagaagaccatctccaaggcaaagggacagcctagggagccacaggtgtacacactgcctccatccagagaggagatgaccaagaaccaggtgtctctgacatgtctggtgaagggcttctatccaagcgacatcgccgtggagtgggagtccaatggccagcccgagaacaattacaagaccacaccccctgtgctggactccgatggctctttctttctgtattctaagctgaccgtggataagagccggtggcagcagggcaacgtgttttcctgctctgtgatgcacgaggccctgcacaatcactacacacagaagagcctgtccctgtctcccggcaagcggagaaagaggggaagcggagagggaagaggctccctgctgacctgtggcgacgtggaggagaacccaggacctatgaagtatctgctgcctacagcagccgccggcctgctgctgctggcagcacagcccgccatggccgacatccagatgacccagagcccttctagcgtgagcgcctccatcggcgacagggtgaccatcacatgccgcgcctcccagggcatcgataactggctgggctggtaccagcagaagcccggcaaggcccctaagctgctgatctatgacgcctctaatctggataccggagtgccaagccggttctctggaagcggctccggcacatactttaccctgacaatctcctctctgcaggccgaggacttcgccgtgtatttttgccagcaggccaaggcattcccacccacctttggcggaggaacaaaggtggacatcaagaggaccgtggcagcaccttccgtgttcatctttcctccatctgatgagcagctgaagagcggcacagcctccgtggtgtgcctgctgaacaatttctacccaagagaggccaaggtgcagtggaaggtggacaacgccctgcagagcggcaattcccaggagtctgtgaccgagcaggacagcaaggattccacatattctctgagctccaccctgacactgagcaaggccgattacgagaagcacaaggtgtatgcctgtgaggtcactcaccaggggctgtcaagtccagtcaccaagtccttcaatcggggggaatgttgataa

SEQ ID NO: 30 - DNA, 2232 bp, Artificial Sequence
  transgenic construct/anti-(ns)hIL-12
ttaagaaaaaatacgggtagaagcagtttgagagctccgttcgccaccatggagttcggcctgtcctgggtgttcctggtggccctgttccgcggcgtgcagtgcgaggtccaattggtcgtggaatcggggggaggattggtacagcccggcgggagcttgcggctgagttgcgcagctagcgggtacagcattactaacgattacgcttggaattgggttagacaggccccaggtaaaggcctcgagtgggtgggttatatctcttactcaggatacacaacgtataatcccagcctcaaatccaggtttaccatctcccgtgatacaagcaaaaatacgctctacctgcaaatgaactctttgagggcagaggacaccgccgtttactattgcgctcgatggacaagtgggcttgactattgggggcagggcacgttagttacagtcagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatacagatgactcagagtccctcctcactctctgcatcagtcggcgaccgggtcactattacttgtaaagcatccgatcttattcacaattggttggcttggtatcaacaaaaaccaggtaaggctcctaaattactcatatacggggctacctcacttgaaacgggagtgcctagccgcttttcaggcagcgggtctggaacagactttaccctgaccatttctagcctgcagccagaagacttcgccacatattactgccaacagtactggacaacgccattcacattcggccaaggtaccaaggtagaaatcaaaaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctga

SEQ ID NO: 31 - DNA, 17430 bp, Newcastle Disease Virus
  Mut HN(F277L)/M(G165W)/F(F117S)/F(F190L)/ F(L289A)/L(V757I)/L(F1551S)/L(R1700L)-Sofituzumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagtttgggctgtcctgggtgttcctggtcgctctgtttaggggggtgcagtgtgaagtgcagctggtcgtcgagtccggggggggcctggtgcagccaggaggcagcctgaggctgtcctgcgcagcatccggctactctatcaccaacgactatgcctggaattgggtgagacaggcaccaggcaagggcctggagtgggtgggctacatctcttatagcggctacaccacatataaccccagcctgaagtccaggttcaccatctctcgcgataccagcaagaacacactgtacctgcagatgaattccctgagggcagaggacaccgccgtgtactattgcgccagatggacaagcggcctggattattggggccagggcaccctggtgacagtgagctccgccagcaccaagggcccttccgtgtttcccctggccccttctagcaagtccacctctggaggaacagccgccctgggctgtctggtgaaggactacttccctgagccagtgaccgtgtcttggaacagcggcgccctgacctccggagtgcacacatttcccgccgtgctgcagtcctctggcctgtacagcctgagctccgtggtgaccgtgccttctagctccctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatcagcagaaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatcccagcgacatcgccgtggagtgggagtccaatggccagcctgagaacaattacaagaccacaccccctgtgctggactccgatggctctttctttctgtatagcaagctgaccgtggataagtcccggtggcagcagggcaacgtgtttagctgctccgtgatgcacgaggccctgcacaatcactacacacagaagtctctgagcctgtccccaggcaagaggagaaagaggggatccggcgagggcagaggctctctgctgacctgtggcgacgtggaggagaaccctggcccaatgaagtatctgctgcctacagcagccgccggcctgctgctgctggcagcacagcctgccatggccgacatccagatgacccagtccccatctagcctgtctgccagcgtgggcgacagggtgaccatcacatgcaaggccagcgatctgatccacaattggctggcctggtaccagcagaagcctggcaaggccccaaagctgctgatctatggagccacctctctggagacaggagtgccaagccgcttctccggatctggaagcggcaccgactttaccctgacaatctcctctctgcagcctgaggatttcgccacatactattgccagcagtactggaccacaccattcacctttggccagggcacaaaggtggagatcaagaggaccgtggccgccccttccgtgttcatctttccaccctctgacgagcagctgaagtccggcacagcctctgtggtgtgcctgctgaacaacttctacccacgcgaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgtgaagtcacccaccaggggctgtcatcaccagtcactaagagtttcaatcggggggaatgctgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 32 - protein, 571 aa, Newcastle disease virus
  HN protein
MDSAVSQVALENDRREANNTWRLVFRIAALLLLVITLAVSAVALAYSMEASTPGDLVSIPTAISRAEERITSALGSNQDVVDRIYKQVALESPLALLNTESIIMNAITSLSYQINGATNNSGCGAPVHDPDYIGGIGQELIVDDTSDVTSFYPSAFQEHLNFIPAPTTGSGCTRIPSFDMSATHYCYTHNVILSGCRDHSHSHQYLALGVLRTSATGRVFFSTLRSINLDDAQNRKSCSVSATPLGCDMLCSKITETEEEDYKSVIPTSMVHGRLGLDGQYHEKDLDVTTLFRDWVANYPGVGGGSFINNRVWFPVYGGLKPSSPSDTAQEGRYVIYKRYNDTCPDEQDYQIRMAKSSYKPRRFGGKRVQQAILSIKVSTSLGEDPVLTVPPNTVALMGAEGRVLTVGTSHFLYQRGSSYFSPALLYPMTVNNKTATLHNPYTFNAFTRPGSVPCQASARCPNSCVTGVYTDPYPLVFHRNHTLRGVFGTMLDDEQARLNPVSAVFDNISRSRITRVSSSSTRAAYTTSTCFKVVKTNKTYCLSIAEISNTLFGEFRIVPLLVEILKDGGV

SEQ ID NO: 33 - protein, 553 aa, Newcastle disease virus
  F protein (F3)
MGPRSSTRIPVPLMLTIRITLALSYVRLTSSLDGRPLAAAGIVVTGDKAVNIYTSSQTGSIIVKLLPNMPKDKEACAKAPLEAYNRTLTTLLTPLGDSIRRIQESVTTSGGRRQRRSIGAIIGSVALGVATAAQITAASALIQANQNAANILRLKESIAATNEAVHEVTGGLSQLAVAVGKMQQFVNDQLNNTAQELDCIKITQQVGVELNLYLTELTTVFGPQITSPALTQLTVQALYNLAGGNVDYLLTKLGVGNNQLSSLIGSGLITGNPIFYDSQTQLLGIQVTAPSVGNLNNMRATYLETLSVSTTKGFASALVPKVVTQVGSVIEELDTSYCIEADLDLYCTRIVTFPMSPGIYSCLSGNTSACMYSRTEGALTTPYMTLKGSVVANCQMTTCRCADPPGIISQNYGEAVSLIDKHSCNVVSLDGITLRLSGEFDATYQKNISILDSQVLVTGNLDISTELGNVNHSISNALDKLEESNSKLDKVNVKLTSTSALITYIVLTVISLVLGMLSLVLACYLMYKQKAQRKTLLWLGNNTLDQMRATTKM

SEQ ID NO: 34 - protein, 364 aa, Newcastle disease virus
  M protein
MDSSRTIGLYFDSALPSSNLLAFPIVLRDVGDGKKQITPQYRIRRLDSWTDSKEDSVFITTYGFIFQVGNEEVTVGMINDNPKRELLSAAMLCLGSVPNVGDPVELARACLTMVVTCKKSATNTERMVFSVVQAPRVLQSCRVVADKYSSVNAVKHVKAPEKIPWSETLEYKVNFVSLTVVPKKDVYKIPTAVLKVSGSSLYNLALNVTIDVEVDPKSPLVKSLSRSDSGYYANLFLHIGLMSTVDKRGKKVTFDQLERKIRRLDLSVGLSDVLGPSVLVKARGARTRLLAPFFSNSGTACYPIANASPQVAKILWSQTACLRSVKIIIQAGTQRAVAVTADHEVTSTKIEKRHTIAKYNPFKK

SEQ ID NO: 35 - protein, 2204 aa, Newcastle disease virus
  L protein
  misc_feature (1717)..(1717): Xaa can be Tyr or His
  misc_feature (1910)..(1910): Xaa can be Glu or Lys
MAGSGPERAEHQIILPESHLSSPLVKHKLLYYWKLTGLPLPDECDFDHLIISRQWKKVLESATPDIERMIKLGRAVHQTLNHNSRITGVLHPRCLEELASIEVPDSTNKFRKIEKKIQIHNTRYGELFTRLCTHVEKKLLGSSWSNNVPRSEEFNSIRTDPAFWFHSKWSTAKFAWLHIKQVQRHLIVAARTRSAVNKLVTLTHKVGQIFVTPELVIVTHTDENKFTCLTQELVLMYADMMEGRDMVSIISSTAAHLRSLSEKIDDILRLVDALARDLGNQVYDVVALMEGFAYGAVQLLEPSGTFAGDFFAFNLQELKDTLTGLLPKDIAESVTHAIATIFSGLEQNQAAEMLCLLRLWGHPLLESRIAAKAVRSQMCAPKMVDFDMILQVLSFFKGTIINGYRKKNAGVWPRVKIDTIYGKVIGQLHADSAEISHDVMLREYKSLSALEFEPCIEYDPVTNLSMFLKDKAIAHPKDNWLASFRRNLLSEDQKKHVREATSTNRLLIEFLESNDFDPYKEMEYLTTLEYLRDDNVAVSYSLKEKEVKVNGRIFAKLTNRLRNCQVMAEGILADQIAPFFQGNGVIQDSISLTKSMLAMSQLSFNSNKKRITDCKERVSSNRNHDPKSKNRRRVATFVTTDLQKYCLNWRYQTVKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAITREVRSDDSPEMVLTQLHQASDNFFKELIHVNHLIGHNLKDRETIRSDTFFIYSKRIFKDGAILSQVLKNSSKLVLISGDLSENTVMSCANIASTVARLCENGLPKDFCYYLNYLMSCVQTYFDSEFSITNNSQLDSNQSWIEDISFVHSYVLTPAQLGGLSNLQYSRLYTRNIGDPGTTAFAEIKRLEAVGLLSPSIMTNILTRPPGNGDWASLCNDPYSFNFETVASPNIVLKKHTQRVLFETCSNPLLSGVHTEDNEAEEKALAEFLLNQEMIHPRVAHAIMEASSVGRRKQIQGLVDTTNTVIKIALTRRPLGIKRLMRIVNYSSMHAMLFRDDVFSSNRSNHPLVSSDMCSQTLADYARNRSWSPLTGGRKILGVSNPDTIELVEGEILSVSGGCTRCDSGDEQFTWFHLPSNIELNDDTSKNPPMRVPYLGSKTQERRAASLAKIAHMSPHVKAALRASSVLIWAYGDNEINWTAALKIARSRCNISSEYLRLLSPLPTAGNLQHRLDDGITQMTFTPASLYRVSPYIHISNDSQRLFTEEGVKEGNVVYQQIMLLGLSLIESLFPMTTTKTYDEITLHLHSKFSCCIREAPVAVPFELLGVAPELRAVTSNKFMYDPSPVSEGDFARLDLAIFKSYELNLESYSTIELMNVLSISSGKLIGQSVVSYDEDTSIKNDAIIVYDNTRNWISEAQNSDVVRLFEYAALEVLLDCSYQLYYLRVRGLNNIVLYMSDLYKNMPGILLSNIAATISHPVIHSRLNAVGLVNHDGSHQLADTDFIEMSAKLLVSCTRRVVSGLHAGNKYDLLFPSVLDDNLSEKMLQLISQLCCLYTVLFATTREIPKIRGLSAEEKCSVLTEYLLSDAVKPLLGSEQVSSIMSPNIVTFPANLYYMSRKSLNLIREREDRDTILALLFPQEPLLEFPLVRDIGARVKDPFTLQPAAFLQELDLSAPARXDAFTISQAHSEHILPNPEEDHLVRYLFRGIGTASSSWYKASHLLSVPEVRCARHGNSLYLAEGSGAIMSLLELHIPHETIYYNTLFSNEMNPPQRHFGPTPTQFLNSVVFRNLQAEVPCKDGFVQEFRPLWRENTEESDLTSDKAVGYITSVVPYRSVSLLHCDIEIPPGSNQSLLDQLATNLSLIAMHSVKXGGVVIIKVLYAMGYYFHLLMNLFTPCSTKGYILSNGYACRGDMECYLIFVMGYLGGPTFVHEVVRMAKTLVQRHGTLLSKSDEITLTRLFTSQQQRVTDILSSPLPRLMKYLRENIDTALIEAGGQPVRPFCAESLVSTLTDMTQTTQIIASHIDTVIRSVIYMEAEGDLADTVFLFTPYNLSMDGKKRTSLKQCTRQILEVTILGLRVKNLNKVGDVISLVLRGMISLEDLLPLRTYLKCSTCPKYLKAVLGITKLKEMFTDASLLYLTRAQQKFYMKTIGNAVKGYYSNCDS

SEQ ID NO: 36 - DNA, 15186 bp, Newcastle disease virus
  Mut HN (F277L)/M(G165W)/F(F117S)/F(F190L)/L(V757I)/L(F1551S)/L(R1700L)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 37 - DNA, 15198 bp, Newcastle disease virus
  Mut HN (F277L)/M(G165W)/F(F117S)/F(F190L)/L(V757I)/L(F1551S)/ L(R1700L)+AfeI
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcgctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 38 - DNA, 17442 bp, Newcastle Disease Virus
  Mut HN (F277L)/M(G165W)/F(F117S)/F(F190L)/L(V757I)/L(F1551S)/L(R1700L)- Lirilumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaagtgcaactggtgcagagcggtgcagaggtgaagaagccaggatcctctgtgaaagtctcatgcaaagccagtggaggtacgttcagcttttacgcaatcagctgggtgagacaggctccgggccaaggtctggaatggatgggcggattcattccaattttcggcgcagcaaactacgcacagaagttccaggggcgggtcacgattactgctgatgagtcgacaagcacagcatacatggagctcagttctttgagaagcgatgatacagcagtttattactgtgctagaattccaagcggttcttattactatgactacgatatggatgtctggggacagggaacaacagtcaccgtatcttccgcgagcaccaagggaccttccgtgtttcccctggccccttgctccagatcaacctcagagtcgactgctgctctcggttgcttggtgaaagattattttccagaacccgttaccgtgtcttggaactccggtgcccttacttcaggggttcataccttccccgcagtcctacagtcatctggtctgtattctctcagctccgtggtcaccgtcccaagcagctcactgggcaccaagacctatacctgcaacgtcgaccataagcctagtaatacaaaggtcgacaagcgagtcgaatccaaatacggacctccttgtccaccttgccccgctcctgaattcctgggtggaccaagcgtctttctgtttccaccaaaacctaaagacacacttatgatttcccgcacacccgaagtcacctgcgtggtggtggatgtcagccaggaagaccctgaggtgcaattcaactggtacgttgatggcgtagaagtccacaacgccaagacaaaacccagagaggagcagtttaatagtacctatagggtggtgtccgtcctgaccgttctccaccaggattggttgaatggcaaggaatacaagtgtaaagtgagcaacaaaggcctaccttcgagcatagaaaagactatctccaaggcaaagggacagccaagggagccccaggtgtacacgctgccaccctctcaggaggagatgactaagaaccaggtgtccttgacctgcctcgtaaaggggttttacccctccgacatagccgtcgaatgggagagtaacggacaacccgagaacaattataaaactacacctcccgtgctcgactctgacggatccttcttcctgtactctaggctgacagtggataaatcccgttggcaggaaggaaatgtattttcctgtagtgtgatgcacgaggctctgcacaatcattatacacagaaatcactgtccttaagcctcggcaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaatcgtcctcacacagagcccggttacgctttcattgtctccaggcgagcgcgctaccctcagctgtagggcgtctcagtccgtttccagctatttagcttggtaccagcaaaaacctggccaggcccctagactgcttatttacgacgcctctaaccgagcgactggtatacccgcaaggttcagcggctcgggctcaggcaccgactttactctcaccatttcatcactggagcctgaggacttcgccgtgtattactgtcagcagaggagtaactggatgtacaccttcggccaagggaccaaattggaaatcaaaaggacagtggccgcgccaagcgtgtttatatttcccccctccgacgaacagttaaagagtggaaccgcatctgtggtatgtctgcttaacaatttttaccctcgcgaggcaaaggttcaatggaaagtcgacaacgccttacagtctggtaacagtcaagagtctgttacggagcaagacagcaaggactccacctattcgctgtcttcaacactaaccctcagcaaggctgattacgagaagcacaaagtttacgcctgtgaggtcacccatcaggggctttctagccctgtcacaaagtcgttcaacaggggagagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 39 - protein, 553 aa, Newcastle disease virus
  F protein (F2)
MGPRSSTRIPVPLMLTIRITLALSYVRLTSSLDGRPLAAAGIVVTGDKAVNIYTSSQTGSIIVKLLPNMPKDKEACAKAPLEAYNRTLTTLLTPLGDSIRRIQESVTTSGGRRQRRSIGAIIGSVALGVATAAQITAASALIQANQNAANILRLKESIAATNEAVHEVTGGLSQLAVAVGKMQQFVNDQLNNTAQELDCIKITQQVGVELNLYLTELTTVFGPQITSPALTQLTVQALYNLAGGNVDYLLTKLGVGNNQLSSLIGSGLITGNPIFYDSQTQLLGIQVTLPSVGNLNNMRATYLETLSVSTTKGFASALVPKVVTQVGSVIEELDTSYCIEADLDLYCTRIVTFPMSPGIYSCLSGNTSACMYSRTEGALTTPYMTLKGSVVANCQMTTCRCADPPGIISQNYGEAVSLIDKHSCNVVSLDGITLRLSGEFDATYQKNISILDSQVLVTGNLDISTELGNVNHSISNALDKLEESNSKLDKVNVKLTSTSALITYIVLTVISLVLGMLSLVLACYLMYKQKAQRKTLLWLGNNTLDQMRATTKM

SEQ ID NO: 40 - DNA, 36 bp, Artificial Sequence
  Primer
acgactcactataggaccaaacagagaatccgtgag

SEQ ID NO: 41 - DNA, 35 bp, Artificial Sequence
  Primer
ccgggaagatccagggcactcttcttgcatgttac

SEQ ID NO: 42 - DNA, 35 bp, Artificial Sequence
  Primer
gggcctgcctcactatggtggtaacatgcaagaag

SEQ ID NO: 43 - DNA, 35 bp, Artificial Sequence
  Primer
tgcatgttaccaccaatgtgtcattgtatcgcttg

SEQ ID NO: 44 - DNA, 37 bp, Artificial Sequence
  Primer
caagaagggagatacgtaatatacaagcgatacaatg

SEQ ID NO: 45 - DNA, 35 bp, Artificial Sequence
  Primer
tcgcttgtatattacttgttgtagcaaagagcacc

SEQ ID NO: 46 - DNA, 38 bp, Artificial Sequence
  Primer
ggcctggatcttcccattatgctgtctgtatacggtgc

SEQ ID NO: 47 - DNA, 38 bp, Artificial Sequence
  Primer
atgccatgccgacccaccaaacaaagacttggtgaatg

SEQ ID NO: 48 - DNA, 24 bp, Artificial Sequence
  Primer
cctatagtgagtcgtattaatttc

SEQ ID NO: 49 - DNA, 20 bp, Artificial Sequence
  Primer
cctggatcttcccgggtcgg

SEQ ID NO: 50 - DNA, 22 bp, Artificial Sequence
  Primer
gggtcggcatggcatctccacc

SEQ ID NO: 51 - DNA, 22 bp, Artificial Sequence
  Primer
gggaagatccaggcctatagtg

SEQ ID NO: 52 - DNA, 35 bp, Artificial Sequence
  Primer
ctctagagtcgacccttctgccaacatgtcttccg

SEQ ID NO: 53 - DNA, 35 bp, Artificial Sequence
  Primer
gggaagcggccgcccgtcggtcagtatccccagtc

SEQ ID NO: 54 - DNA, 37 bp, Artificial Sequence
  Primer
ctctagagtcgaccccagagtgaagatggccaccttc

SEQ ID NO: 55 - DNA, 35 bp, Artificial Sequence
  Primer
gggaagcggccgcccgtagtagtgatcagccattc

SEQ ID NO: 56 - DNA, 35 bp, Artificial Sequence
  Primer
ctctagagtcgacccgggtaggacatggcgggctc

SEQ ID NO: 57 - DNA, 37 bp, Artificial Sequence
  Primer
gggaagcggccgccctgcctttaagagtcacagttac